(19) United States
(12) Patent Application Publication
(10) Pub. No.: US 2026/0199495 A1
(43) Pub. Date: Jul. 16, 2026

(54) Lysosome-Mediated Degradation of Extracellular Proteins

(71) Applicant: Washington University, St. Louis, MO (US)

(72) Inventors: Gilbert Gallardo, St. Louis, MO (US); Aisan Farhadi Gharehgheshlaghi, St. Louis, MO (US); Shreedarshanee Devi Shamulailatpam, St. Louis, MO (US); Dia Yahng, St. Louis, MO (US); Sofia Luella Heese, St. Louis, MO (US)

(73) Assignee: Washington University, St. Louis, MO (US)

(21) Appl. No.: 19/353,604

(22) Filed: Oct. 8, 2025

Related U.S. Application Data
(60) Provisional application No. 63/704,935, filed on Oct. 8, 2024.

(51) Int. Cl. A61K 47/66; A61K 35/17; A61K 40/11; A61K 40/31; A61K 47/68; A61P 25/28; A61P 37/06; C07K 14/705; C07K 14/71; C07K 16/18; C07K 16/24; C12N 15/86

(52) U.S. Cl. CPC A61K 47/665; A61K 35/17; A61K 40/11; A61K 40/31; A61K 47/6845; A61P 25/28; A61P 37/06; C07K 14/7056; C07K 14/71; C07K 16/18; C07K 16/24; C12N 15/86; C07K 2317/569; C07K 2319/00; C12N 2750/14143; C12N 2830/50

(57) ABSTRACT

Among the various aspects of the present disclosure is the provision of targeting a secreted protein for degradation via the lysosome. Disclosed herein is a composition of a lysosomal degrading chimeric receptor (LDCR). A method to treat diseases by targeting a secreted protein to the lysosome using LDCRs is also disclosed.

### CROSS-REFERENCE TO RELATED APPLICATIONS

This application claims priority from U.S. Provisional Application Ser. No. 63/704,935 filed on Oct. 8, 2024, which is incorporated herein by reference in its entirety.

### STATEMENT REGARDING FEDERALLY SPONSORED RESEARCH OR DEVELOPMENT

This invention was made with government support under AG0063817 awarded by the National Institutes of Health (NIH). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “021108-US-NP_Sequence-listing” created on 8 Oct. 2025; 55,051 bytes). The subject matter of the Sequence Listing is incorporated herein by reference in its entirety.

### FIELD OF THE DISCLOSURE

The present disclosure generally relates to targeted protein degradation compositions and related methods of use thereof.

### BACKGROUND

Targeted protein degradation (TPD) via lysosome provided new tools which can selectively target disease-causing proteins for degradation, including proinflammatory proteins that regulate neuroinflammation and progress several neurodegenerative diseases. Although how neuroinflammation is regulated remains unclear, certain proinflammatory proteins including, tumor necrosis factor-α (TNFα), Apolipoprotein 4 (ApoE4), interleukin-6 (IL-6), interferon gamma (IFN-γ), and lipocalin 2 (LCN2), have been shown to progress several neurodegenerative diseases, including Alzheimer's disease (AD). The APOE4 gene encoding ApoE4 is the highest genetic risk factor for late-onset AD, expressed in over half of AD patients. In amyloidosis and tauopathy mouse models, expressing ApoE4 exacerbates neuroinflammatory and AD pathologies, and astrocytic ApoE4 knockout decreases AD pathologies. Similarly to ApoE4, TNFα levels increasingly correlate with disease progression in AD patients and mouse models of AD pathologies. In addition, global TNFα receptor knockout or anti-TNFα antibodies have shown protective effects in decreasing amyloid plaques and tau pathology in mouse models of AD pathologies6-10. Although therapeutic strategies, including antisense oligonucleotides (ASO) or antibodies targeting ApoE4/TNF-α, have variable degrees of protection in mouse models, they face limitations, including low or impermeability to the blood-brain barrier (BBB), and ASOs lack cell-type specificity. Additionally, neutralizing TNFα peripherally in the aging AD population may compromise the immune system. Another strategy to lower ApoE4 levels in the brain was generating a transgenic mouse line overexpressing ApoE's low-density lipoprotein receptor (LDLR) that mediates ApoE lysosomal degradation. Apart from neuroinflammation, neurodegenerative diseases are also initiated or progressed by several extracellular pathological proteins, including amyloid-β(Aβ) and tau protein in Alzheimer's disease (AD), α-synuclein in Parkinson's disease, which serve as extracellular therapeutic targets for TPD strategies.

In addition, several extracellular therapeutic targets, such as cytokines, are pivotal in immune-mediated inflammatory diseases (IMID). IMIDs are a diverse group of diseases where elevated cytokines promote chronic inflammation and are among the most common diseases, including rheumatoid arthritis, psoriasis, and inflammatory bowel diseases. Conventionally, extracellular cytokines, including tumor necrosis factor-α (TNFα) and interleukin-6 (IL-6), have been targeted with monoclonal antibodies (mAb) that neutralize their receptor binding. Although beneficial, monoclonal antibody therapies may induce complement-dependent cytotoxicity, initiated by C1q binding to the Fc regions of antibodies, leading to the formation of the membrane attack complex, pore formation, and cell lysis. In addition, long-term monoclonal antibody treatment may also lead to immunogenicity, resulting in the formation of anti-drug antibodies (ADA), decreased efficacy, and potentially the development of autoimmune conditions.1

In addition to IMIDs, autoantibody diseases are also a diverse group of chronic diseases involving the production of aberrant extracellular autoantibodies targeting cellular antigens on tissues and organs. The pathological mechanisms of autoantibodies are also diverse, differing in each disease. In Graves' disease, autoantibodies are agonistic, leading to activation of the thyrotropin receptor and overproduction of thyroid hormone. Autoantibodies' pathophysiological mechanism can also be antagonistic, as in Myasthenia gravis (MG), targeting acetylcholine receptors (AChR) or membrane protein muscle-specific kinase, leading to muscle weakness and fatigue. Autoantibodies may also mediate disease by forming immune complexes that mediate complement-dependent cytotoxicity, which is evident in haemolytic anemia, leading to a decrease in red blood cells. The targeting of autoantibodies per se has largely been limited and current therapies have resorted to non-specific B-cell depletion strategies. However, chronic B-cell depletion may compromise the immune system, leading to severe and potentially life-threatening infections. These concerns are of particular importance for autoantibody diseases and IMIDs, which are chronic diseases necessitating long-term treatment. More recently, new strategies targeting autoantibodies have been developed through chimeric antigen receptor (CAR) T cells. CARs are engineered chimeric fusion proteins consisting of an antigen-binding domain, a hinge region-transmembrane domain, a co-stimulatory domain, and a T cell activation domain derived from CD3ζ. CAR T cells targeting specific antigens expressed on tumor cells demonstrated profound cytotoxic effects on tumor growth and led to the development of a chimeric autoantibody receptor (CAAR). CAARs consist of an autoantigen chimeric fusion protein that targets T cell cytotoxicity toward B cells expressing autoantibodies. While CAARs provide a new approach for targeting autoantibody diseases, they are not without potential adverse effects. Major drawbacks of all CAR therapies containing a CD3 (active domain include cytokine-release syndrome (CRS) and immune effector cell-associated neurotoxicity (ICANS). Therefore, there is an unmet need to improve therapeutic strategies that reduce potential adverse effects and improve efficacy when targeting proinflammatory proteins or autoantibodies.

### SUMMARY OF THE INVENTION

Among the various aspects of the present disclosure is the provision of targeted protein degradation compositions and methods of use thereof.

Briefly, therefore, the present disclosure is directed to the use of lysosomal degrading chimeric receptors (LDCRs) to target extracellular secreted therapeutic proteins to the lysosome for degradation.

In one aspect, a composition for degrading a target protein in a patient, the composition comprising a lysosomal degrading chimeric receptor (LDCR) is disclosed. The LDCR includes an engineered mannose 6-phosphate receptor (MPR), the engineered MPR comprising an MPR with a targeting moiety fused at least one of a transmembrane domain, a cytoplasmic domain, and any combination thereof; or an engineered asialoglycoprotein receptor (ASGPR), the engineered ASGPR comprising an ASGPR with the targeting moiety fused at least one of an extracellular stalk region and a carbohydrate recognition binding domain, and any combination thereof; wherein the targeting moiety is configured to bind to the target protein. In some aspects, the targeting moiety comprises a nanobody targeting the target protein or an autoantibody epitope sequence targeting the target protein. In some aspects, the composition further includes the LDCR incorporated into an adeno-associated virus or a chimeric antigen receptor T-cell therapy. In some aspects, the target protein is selected from one of a ligand-binding domain, an autoantigen peptide sequence encoding an autoantibody, a single-chain variable fragment (scFV), or a nanobody. In some aspects, the ligand-binding domain is configured to selectively bind to a chemokine or a cytokine. In some aspects, the autoantigen encoded by the autoantigen peptide sequence is configured to selectively bind to an autoantibody. In some aspects, the nanobody is configured to selectively bind to a chemokine, a cytokine, or an extracellular therapeutic pathological proteins. In some aspects, the LDCR is encoded by a nucleotide sequence selected from the group consisting of SEQ ID NOS. 1, 3, 5, 7, 9, 11, 13, 15, 17, 19, 21, 23, 25, 27, 29, 31, 33, 35, 37, or 39. In some aspects, the LDCR comprises a peptide sequence selected from the group consisting of SEQ ID NOS. 2, 4, 6, 8, 10, 12, 14, 16, 18, 20, 22, 24, 26, 28, 30, 32, 34, 36, 38, or 40.

In another aspect, a method of treating a disease or disorder in a patient by targeting a secreted protein for degradation via a lysosome is disclosed. The method includes administering a composition comprising a therapeutically effective amount of a lysosomal degrading chimeric receptor (LDCR) to the patient the LDCR comprising: an engineered mannose 6-phosphate receptor (MPR), the engineered MPR comprising an MPR with a targeting moiety fused at least one of a transmembrane domain, a cytoplasmic domain, and any combination thereof; or an engineered asialoglycoprotein receptor (ASGPR), the engineered ASGPR comprising an ASGPR with the targeting moiety fused at least one of an extracellular stalk region and a carbohydrate recognition binding domain, and any combination thereof; wherein the targeting moiety is configured to bind to the target protein. In some aspects, the method further includes administering the therapeutically effective amount of the LDCR using a method selected from adeno-associated virus (AAV) and chimeric antigen receptor T-cell therapy. In some aspects, the disease is an autoimmune disease or Alzheimer's Disease (AD). In some aspects, the secreted protein targeted by the nanobody is selected from an epitope from a tau-5 protein, an epitope from a Apoe4 protein, or an epitope from a myelin-based protein. In some aspects, the targeting moiety comprises a nanobody targeting the target protein or an autoantibody epitope sequence targeting the target protein. In some aspects, the target protein is selected from one of a ligand-binding domain, an autoantigen peptide sequence encoding an autoantibody, a single-chain variable fragment (scFV), or a nanobody. In some aspects, the ligand-binding domain is configured to selectively bind to a chemokine or a cytokine. In some aspects, the autoantigen encoded by the autoantigen peptide sequence is configured to selectively bind to an autoantibody. In some aspects, the LDCR is encoded by a nucleotide sequence selected from the group consisting of SEQ ID NOS. 1, 3, 5, 7, 9, 11, 13, 15, 17, 19, 21, 23, 25, 27, 29, 31, 33, 35, 37, or 39. In some aspects, the LDCR comprises a peptide sequence selected from the group consisting of SEQ ID NOS. 2, 4, 6, 8, 10, 12, 14, 16, 18, 20, 22, 24, 26, 28, 30, 32, 34, 36, 38, or 40.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Schematic for engineering the Lysosomal Degrading Chimeric Receptor (LDCR) utilizing the transmembrane and cytoplasmic domains of the CI-MPR fused to an anti-GFP nanobody or antigen epitope sequence for an antibody known as anti-tau5 mAB epitope sequence to demonstrate proof of concept.

FIG. 2. Schematic for engineering the Lysosomal Degrading Chimeric Receptor (LDCR) utilizing the transmembrane and cytoplasmic domains of the CD-MPR fused to an anti-GFP nanobody or antigen epitope sequence for an antibody known as tau5 mAB to demonstrate proof of concept for targeting extracellular proteins or autoantibodies.

FIG. 3. Schematic for engineering the Lysosomal Degrading Chimeric Receptor (LDCR) utilizing the transmembrane and cytoplasmic domains of the ASGPR fused to an anti-GFP nanobody or antigen epitope sequence for an antibody known as tau5 mAB to demonstrate proof of concept for targeting extracellular proteins or autoantibodies.

FIG. 4 is a schematic diagram depicting the LDCR trafficking from trans-Golgi to the cell surface and lysosome. EE: early endosome, LE: late endosome Lyso: lysosome.

FIG. 5A: The ligand-binding domain of the MPRs is not necessary for lysosomal trafficking. GFP fused to the transmembrane domains of the MPRs traffic to the cell surface and localizes with lysosomal marker LAMP1. The orange arrow points to the lower surface localization and the red arrow points to the accumulation following Dynasore treatment. Scale bar 10 um.

FIG. 5B: GFP fused to the cytoplasmic domains of the MPRs traffic to the cell surface and localizes with lysosomal marker LAMP1. The orange arrow points to the lower surface localization and the red arrow points to the accumulation following Dynasore treatment. Scale bar 10 um.

FIG. 6: The anti-GFP nanobody fused to the transmembrane-cytoplasmic domains of the MPRs binds to extracellular GFP protein. The anti-GFP LDCRi and LDCRsi binds to extracellular GFP and localized with the HA tag and lysosomal maker LAMP1. Scale bar 10 um.

FIG. 7A: Secretory N-terminal sequence increases cell surface localization. Dynasore treatment increased GFP localized at the membrane for LDCRsi relative to Dynasore treatment of the LDCRci.

FIG. 7B: Quantification of GFP localized at the membrane for LDCRsi relative to Dynasore treatment of the LDCRci. Data are mean±s.e.m. student t-test n=70 cells and 3 biological replicates. Normalized to LDCRci and LDCRcd. ***p<0.001.

FIG. 7C: Dynasore treatment increased GFP localized at the membrane for LDCRsd relative to Dynasore treatment of the LDCRcd. Scale bar 20 um.

FIG. 7D: Quantification of GFP localized at the membrane for LDCRsd relative to Dynasore treatment of the LDCRcd. Data are mean±s.e.m. student t-test n=70 cells and 3 biological replicates. Normalized to LDCRci and LDCRcd. ****p<0.0001.

FIG. 8A: Anti-GFP LDCRs bind to extracellular GFP protein. LDCRci and LDCRsi increase binding to extracellular GFP protein in at time dependent manner. Scale bar 20 μm.

FIG. 8B is a set of graphs quantifying LDCRci and LDCRsi binding to extracellular GFP protein over time. All data are mean±s.e.m. one way ANOVA n=70 and 3 biological replicates. **p<0.01,****p<0.0001.

FIG. 8C is a set of immunofluorescent images of LDCRcd and LDCRsd increase binding to extracellular GFP protein in a time dependent manner. Scale bar 20 μm.

FIG. 8D is a set of graphs quantifying LDCRcd and LDCRsd binding to extracellular GFP protein over time. All data are mean±s.e.m. one way ANOVA n=70 and 3 biological replicates. *p<0.5, ****p<0.0001.

FIG. 9A is a schematic illustrating treatment paradigm showing anti-GFP LDCRci and LDCRsi traffic extracellular GFP protein to the lysosome.

FIG. 9B: is a set of immunofluorescent images of LDCRci and LDCRsi treated with DMSO or lysosomal inhibitor bafilomycin, which led to an accumulation of intracellular GFP intensity. Scale bar 20 μm.

FIG. 9C: is a set of graphs quantifying LDCRci and LDCRsi treated with DMSO or lysosomal inhibitor bafilomycin from FIG. 9B. data are mean±s.e.m. one way ANOVA n=70 and 3 biological replicates, **p<0.01,***p<0.001.

FIG. 9D: is a set of immunofluorescent images following lysosomal inhibition with bafilomycin. The cells were washed and imaged over time and demonstrated a time-dependent decrease in GFP intensity quantified in e. arrowheads illustrate GFP degradation in a time dependent manner. Scale bar 20 μm.

FIG. 9E: is a set of graphs quantifying GFP intensity in cells treated with bafilomycin or DMSO over time. Data are mean±s.e.m. one way ANOVA n=70 and 3 biological replicates *p<0.05, **p<0.01,***p<0.001,****p<0.0001.

FIG. 10A is a schematic illustrating treatment paradigm showing anti-GFP LDCRcd and LDCRsd traffic extracellular GFP protein to the lysosome.

FIG. 10B is a set of immunofluorescent images of LDCRcd and LDCRsd treated with DMSO or lysosomal inhibitor bafilomycin, which led to an accumulation of intracellular GFP intensity. Scale bar 20 μm.

FIG. 10C is a set of graphs quantifying GFP intensity in LDCRcd and LDCRsd cells treated with DMSO or lysosomal inhibitor bafilomycin. Data are mean±s.e.m. one way ANOVA n=70 and 3 biological replicates.

FIG. 10D is a set of immunofluorescent images following lysosomal inhibition with bafilomycin the cells were washed and imaged over time and demonstrated a time-dependent decrease in GFP intensity. Arrowheads illustrate GFP degradation in a time-dependent manner.

FIG. 10E is a set of graphs quantifying GFP intensity in cells treated with DMSO or bafilomycin over time. Data are mean±s.e.m. one way ANOVA n=70 and 3 biological replicates *p<0.05, **p<0.01.

FIG. 11A is a schematic depicting GFP-pHrodoRed binds to anti-GFP LDCRs and fluoresces at an acidic pH indicating lysosomal trafficking.

FIG. 11B is a set of immunofluorescent images of LDCRci and LDCRsi binding extracellular GFP-pHrodoRed and an increase in red fluorescent intensity, indicting extracellular GFP is trafficked to the lysosomes. Scale bar 10 um.

FIG. 11C is a set of immunofluorescent images of LDCRcd and LDCRsd binding extracellular GFP-pHrodoRed and an increase in red fluorescent intensity, indicting extracellular GFP is trafficked to the lysosomes. Scale bar 10 um.

FIG. 12A is a schematic for treatment paradigm for LDCRs endocytosis extracellular GFP protein and degrade intracellularly.

FIG. 12B is an immunoblot image of HEK293t cell lysates for LDCRci and LDCRsi which revealed a time-dependent decrease in GFP protein.

FIG. 12C is an immunoblot image of HEK293t cell lysates for LDCRcd and LDCRsd which revealed a time-dependent decrease in GFP protein.

FIG. 13A is a graph of an ELISA-based analysis for enrichment VHH binding to E4. Anti-human E4 VHH binds to human E4 protein.

FIG. 13B is a graph of an ELISA-based analysis for Phage display identified four clones.

FIG. 13C is a graph of an ELISA-based analysis validating the four VHH binding to human E4 data.

FIG. 14: Different LDCRs generated from the MPRs uptaked GFP from conditioned media. Immunoblot data showed decrease of GFP level in condition media compared to negative control.

FIG. 15A is a set of immunofluorescent images showing the LDCR generated from the ASGPR transmembrane-cytoplasmic domain fused to an anti-GFP nanobody referred to as LDCRag binds to extracellular GFP protein in the conditioned media in a time-dependent manner. Scale bar 20 um.

FIG. 15B is a graph quantifying LDCRag binding to extracellular GFP protein in the conditioned media in a time-dependent manner. Data are mean±s.e.m. One-way ANOVA, n=105 cells and 3 biological replicates *p<0.05, ***p<0.001.

FIG. 15C is a set of immunofluorescent images showing LDCRag binding extracellular GFP-pHrodoRed and leads to an increase in red fluorescent intensity, indicating extracellular GFP is trafficked to the lysosomes. Scale bar 20 um.

FIG. 15D is a graph quantifying LDCRag binding extracellular GFP-pHrodoRed. Data are mean±s.e.m. One-way ANOVA, n=105 cells and 3 biological replicates *p<0.05.

FIG. 16A is a set of immunofluorescent images of LDCRag treated with GFP protein in conditioned media followed by IF analysis for LDCRag (anti-HA) and GFP, demonstrating co-localization. Scale bar 20 um.

FIG. 16B is a graph of Pearson's coefficient between GFP colocalized with HA. Data are mean±s.e.m. n=3 biological replicates.

FIG. 16C is a set of immunofluorescent images of LDCRag treated with GFP protein in conditioned media, followed by IF analysis for GFP and lysosomal marker LAMP1, demonstrating co-localization. Scale bar 20 um.

FIG. 16D is a graph of Pearson's coefficient between GFP colocalized with LAMP1. Data are mean±s.e.m. n=3 biological replicates.

FIG. 16E is a set of immunofluorescent images of LDCRag treated with GFP protein in conditioned media, followed by IF analysis for GFP and early endosomal marker EEA1 demonstrating less co-localization. Scale bar 20 um.

FIG. 16F is a graph of Pearson's coefficient between GFP colocalized with EEA1. Data are mean±s.e.m. n=3 biological replicates.

FIG. 17A is a set of immunofluorescent images of LDCRag, treated with GFP protein in conditioned media, followed by live-imaging demonstrating a significant increase in GFP fluorescent intensity at 24 hours, followed by a significant decrease at 48 hours. At 48 hours, GFP protein was replenished in the conditioned media, leading to a significant increase in GFP fluorescent intensity at 72 hours. At 96 hours HEPA-LDCRag displayed a significant decrease in GFP fluorescent intensity. Negative controls do not display any GFP fluorescent intensity at any time point. Scale bar 20 um

FIG. 17B is a set of graphs quantifying GFP protein intensity. All data are mean±s.e.m. analyzed by one way ANOVA. n=60 cells from 3 biological replicates **p<0.01, ****p<0.0001.

FIG. 18A is a schematic for treatment paradigm for GFP depletion.

FIG. 18B is an image of an immunoblot of conditioned media depicting a time-dependent decrease in extracellular GFP protein in LDCRag and no change in the negative control.

FIG. 18C is a set of graphs quantifying GFP expression from the immunoblot in FIG. 18B. Data are mean±s.e.m. one-way ANOVA and 2-4 biological replicates *p<0.05, ****p<0.0001

FIG. 18D is a schematic for treatment paradigm for GFP rate of degradation.

FIG. 18E is an image of an immunoblot showing a negative control of cell lysates depicting no GFP.

FIG. 18F is a graph quantifying GFP expression from the immunoblot in FIG. 18E. Data are mean±s.e.m. one-way ANOVA and 2-4 biological replicates.

FIG. 18G is an image of an immunoblot showing LDCRag cell lysate depicting a time-dependent decrease in intracellular GFP protein.

FIG. 18H is a graph quantifying GFP expression from the immunoblot in FIG. 18G. Data are mean±s.e.m. one-way ANOVA and 2-4 biological replicates.

FIG. 19A is a set of immunofluorescent images of LDCRag treated with DMSO or lysosomal inhibitor bafilomycin led to an accumulation of intracellular GFP intensity. Scale bar 20 μm

FIG. 19B is a graph quantifying GFP intensity in cells treated with DMSO or bafilomycin from FIG. 19A.

FIG. 19C is an image of an immunoblot of cell lysate depicting an accumulation of the GFP in bafilomycin-treated group compared to DMSO control at 24 hrs.

FIG. 19D is a graph quantifying the accumulation of the GFP in bafilomycin-treated group compared to DMSO control at 24 hrs.

FIG. 20 is a schematic depicting an LDCR fused the fused to the extracellular ligand-binding domain of the TNFα receptor TNF-R2, which is refer to as LDCRagTNFα.

FIG. 21A is a set of immunofluorescent images of LDCRagTNFα treated with TNFα in conditioned media followed by IF analysis for LDCRag (anti-HA) and GFP, demonstrating co-localization. Scale bar 20 um.

FIG. 21B is a graph of Pearson's coefficient between TNFα colocalized with HA. Data are mean±s.e.m., n=3 biological replicates.

FIG. 21C is a set of immunofluorescent images of LDCRag treated with TNFα in conditioned media, followed by IF analysis for TNFα and lysosomal marker LAMP1, demonstrating co-localization. Scale bar 20 um.

FIG. 21D is a graph of Pearson's coefficient between TNFα colocalized with LAMP1. Data are mean±s.e.m., n=3 biological replicates.

FIG. 21E is a set of immunofluorescent images of LDCRag were treated with TNFα in conditioned media, followed by IF analysis for TNFα and early endosomal marker EEA1, demonstrating co-localization.

FIG. 21F is a graph of Pearson's coefficient between TNFα colocalized with EEA1. Data are mean s.e.m., n=3 biological replicates.

FIG. 22A is a schematic for a TNFα treatment paradigm for conditioned media analysis.

FIG. 22B is an image of an immunoblot of conditioned media depicting a time-dependent decrease in extracellular TNFα in LDCRagTNFα and no change in the negative control.

FIG. 22C is a set of graphs quantifying TNFα from the immunoblot of FIG. 22B. Data are mean±s.e.m. one-way ANOVA and 2-4 biological replicates; ****p<0.0001.

FIG. 22D is a schematic for a TNFα treatment paradigm for lysate analysis.

FIG. 22E is an image of an immunoblot of LDCRagTNFα immunoblots of cell lysate depicting a time-dependent decrease in intracellular TNFα protein.

FIG. 22F is a graph quantifying TNFα from the immunoblot of FIG. 22E. Data are mean±s.e.m. one-way ANOVA and 2-4 biological replicates *p<0.05, ****p<0.0001.

FIG. 22G is an image of an immunoblot of a negative control immunoblots of cell lysate depicting no TNFα.

FIG. 22H is a graph quantifying TNFα from the negative control immunoblot of FIG. 22G. Data are mean±s.e.m. one-way ANOVA and 2-4 biological replicates.

FIG. 23A is a set of immunofluorescent images of LDCRagTNFα treated with DMSO or lysosomal inhibitor bafilomycin increases TNFα intracellularly time-dependently. Scale bar 20 μm.

FIG. 23B is a graph quantifying the immunofluorescent images of FIG. 23A. Data are mean±s.e.m.

FIG. 24A is a set of immunofluorescent images of LDCRagTNFα expressing astrocytes and controls treated with TNFα in conditioned media, followed by IF analysis for astrocytic marker glial fibrillary acidic protein (GFAP), LDCRag (anti-HA) and TNFα, demonstrating co-localization. Primary astrocytes expressing LDCRagTNFα depletes extracellular TNFα levels by lysosomal trafficking. Scale bar 20 um.

FIG. 24B is a set of immunofluorescent images of LDCRagTNFα colocalizes TNFα with the lysosomes (LAMP1) in astrocytes. Scale bar 20 um.

FIG. 24C is a schematic and corresponding immunoblot f conditioned media depicting a significant decrease in extracellular TNFα collected from LDCRagTNFα expressing astrocytes and no change in the negative control.

FIG. 24D is a graph quantifying TNFα from the immunoblot in FIG. 24C. Data are mean±s.e.m. Student's t-test. ***p<0.001.

FIG. 25A is a schematic for AAV administration to express the LDCRag TNFα in hepatocytes to reduce circulating TNFα following acute inflammation with an LPS injection, and collection of blood.

FIG. 25B is a set of immunoblots of liver lysates treated as described in FIG. 25A. The liver lysates demonstrate expression of the LDCRagTNFα and negative control.

FIG. 25C is a graph of ELISA analysis of serum collected time dependently demonstrates that the expression of LDCRagTNFα reduces circulating TNFα in LPS-injected mice.

FIG. 26A is a schematic illustration of mice LDCRag, a lysosomal degrading chimeric receptor (LDCR) modified from ASGPR1 in accordance with one aspect of the disclosure.

FIG. 26B is a nucleotide sequence encoding mice LDCRag shown schematically in FIG. 26A.

FIG. 26C is an amino acid sequence of mice LDCRag shown schematically in FIG. 26A.

FIG. 27A is a nucleotide sequence encoding the mice LDCRag of FIG. 17B modified to bind to the extracellular TNF alpha. LDCRag in the extracellular part contains the ligand-binding domain of the TNFR2 receptor (4 domains) that binds to TNF alpha.

FIG. 27B is an amino acid sequence of mice LDCRag expressed by the nucleotide sequence of FIG. 27A.

FIG. 28A is a schematic illustration of human LDCRag, a lysosomal degrading chimeric receptor (LDCR) modified from ASGPR1 in accordance with one aspect of the disclosure.

FIG. 28B is a nucleotide sequence encoding human LDCRag shown schematically in FIG. 28A.

FIG. 28C is an amino acid sequence of human LDCRag shown schematically in FIG. 28A.

FIG. 29A is a nucleotide sequence encoding the human LDCRag of FIG. 28B modified to bind to the extracellular TNF alpha. LDCRag in the extracellular part contains the ligand-binding domain of the TNFR2 receptor (4 domains) that binds to TNF alpha.

FIG. 29B is an amino acid sequence of human LDCRag expressed by the nucleotide sequence of FIG. 29A.

FIG. 30A is a schematic illustration of mice LDCRci receptor, a lysosomal degrading chimeric receptor (LDCR) modified from MPRci receptor (type 1) in accordance with one aspect of the disclosure.

FIG. 30B is a nucleotide sequence encoding mice LDCRci shown schematically in FIG. 30A.

FIG. 30C is an amino acid sequence of mice LDCRci shown schematically in FIG. 30A.

FIG. 31A is a nucleotide sequence encoding the mice LDCRci of FIG. 30B modified to bind to the extracellular target with a nanobody.

FIG. 31B is an amino acid sequence of mice LDCRci expressed by the nucleotide sequence of FIG. 31A.

FIG. 32A is a schematic illustration of mice LDCRsi, a modified mice MPRci receptor with modified secretory signal peptide to increase cell surface expression of the receptor, named MPRsi, in accordance with one aspect of the disclosure.

FIG. 32B is a nucleotide sequence encoding mice LDCRsi shown schematically in FIG. 32A.

FIG. 32C is an amino acid sequence of mice LDCRsi shown schematically in FIG. 32A.

FIG. 33A is a nucleotide sequence encoding the mice LDCRsi of FIG. 33B modified to bind to the extracellular target with a nanobody.

FIG. 33B is an amino acid sequence of mice LDCRci expressed by the nucleotide sequence of FIG. 33A.

FIG. 34A is a schematic illustration of human LDCRci, a modified MPRci receptor, in accordance with one aspect of the disclosure.

FIG. 34B is a nucleotide sequence encoding human LDCRsi shown schematically in FIG. 34A.

FIG. 34C is an amino acid sequence of human LDCRsi shown schematically in FIG. 34A.

FIG. 35A is a nucleotide sequence encoding the human LDCRci of FIG. 25A fused to TNFR2 ligand binding domains designed to bind to the extracellular TNF alpha.

FIG. 35B is an amino acid sequence of human LDCRci expressed by the nucleotide sequence of FIG. 35A.

FIG. 36A is a schematic illustration of human LDCRsi, a modified MPRci receptor with modified secretory signal peptide to increase cell surface expression of the receptor, named MPRsi, in accordance with one aspect of the disclosure.

FIG. 36B is a nucleotide sequence encoding human LDCRsi shown schematically in FIG. 36A.

FIG. 36C is an amino acid sequence of human LDCRsi shown schematically in FIG. 36A.

FIG. 37A is a nucleotide sequence encoding the human LDCRci of FIG. 25A fused to TNFR2 ligand binding domains designed to bind to the extracellular TNF alpha.

FIG. 37B is an amino acid sequence of human LDCRci expressed by the nucleotide sequence of FIG. 37A.

FIG. 38A is a schematic illustration of mice LDCRcd, a modified mice MPRcd receptor (type 1), in accordance with one aspect of the disclosure.

FIG. 38B is a nucleotide sequence encoding mice LDCRcd shown schematically in FIG. 38A.

FIG. 38C is an amino acid sequence of mice LDCRcd shown schematically in FIG. 38A.

FIG. 39A is a nucleotide sequence encoding the mice LDCRcd of FIG. 29B modified to bind to the extracellular target with a nanobody.

FIG. 39B is an amino acid sequence of mice LDCRcd expressed by the nucleotide sequence of FIG. 30A.

FIG. 40A is a schematic illustration of mice LDCRsd, a modified mice MPRsd receptor (type 1) with a modified secretory signal peptide to increase cell surface expression of the receptor named MPRsd, in accordance with one aspect of the disclosure.

FIG. 40B is a nucleotide sequence encoding mice LDCRsd shown schematically in FIG. 40A.

FIG. 40C is an amino acid sequence of mice LDCRsd shown schematically in FIG. 40A.

FIG. 41A is a nucleotide sequence encoding the mice LDCRsd of FIG. 31B modified to bind to the extracellular target with a nanobody.

FIG. 41B is an amino acid sequence of mice LDCRsd expressed by the nucleotide sequence of FIG. 41A.

FIG. 42A is a schematic illustration of human LDCRcd, a modified human MPRcd receptor (type 1), in accordance with one aspect of the disclosure.

FIG. 42B is a nucleotide sequence encoding human LDCRcd shown schematically in FIG. 42A.

FIG. 42C is an amino acid sequence of human LDCRcd shown schematically in FIG. 42A.

FIG. 43A is a nucleotide sequence encoding the human LDCRcd of FIG. 33B fused to TNFR2 ligand binding domains designed to bind to the extracellular TNF alpha.

FIG. 43B is an amino acid sequence of human LDCRcd fused to TNFR2 ligand binding domains designed to bind to the extracellular TNF alpha expressed by the nucleotide sequence of FIG. 43A.

FIG. 44A is a schematic illustration of human LDCRsd, a modified mice MPRsd receptor (type 1) with a modified secretory signal peptide to increase cell surface expression of the receptor named MPRsd, in accordance with one aspect of the disclosure.

FIG. 44B is a nucleotide sequence encoding human LDCRsd shown schematically in FIG. 44A.

FIG. 44C is an amino acid sequence of human LDCRsd shown schematically in FIG. 44A.

FIG. 45A is a nucleotide sequence encoding the human LDCRsd of FIG. 35B modified to bind to extracellular Tnf-V alpha.

FIG. 45B is an amino acid sequence of human LDCRsd expressed by the nucleotide sequence of FIG. 45A.

FIG. 46 is an AAV plasmid map used to transduce the lysosomal degrading chimeric receptors (LDCRs) into cells in accordance with some aspects of the disclosure.

### DETAILED DESCRIPTION

The present disclosure is based, at least in part, on the discovery of lysosomal degrading chimeric receptors (LDCRs) and methods of use as a therapeutic by targeting extracellular proteins of interest to the lysosome for degradation.

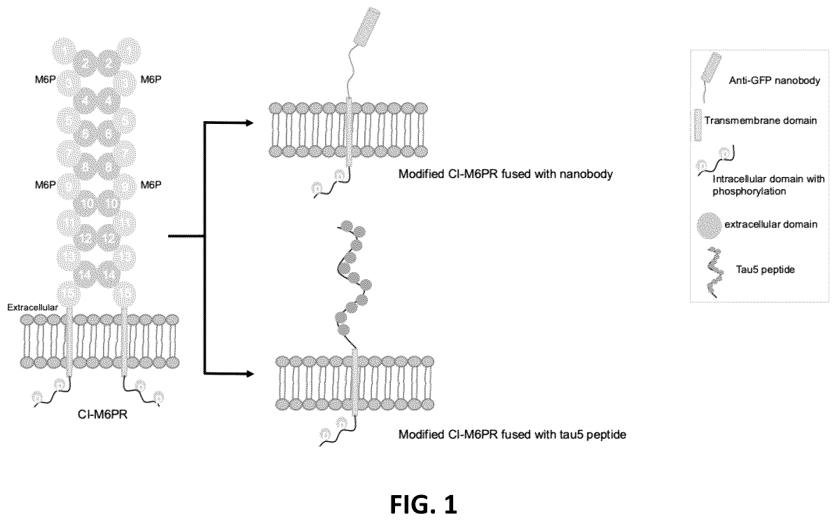

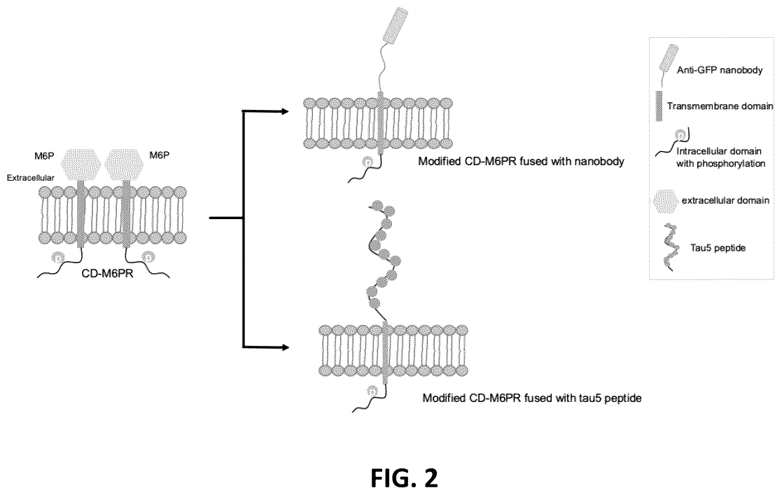

In some aspects, this mechanism for targeting extracellular proteins or autoantibodies for degradation involved the reengineering of the mannose 6-phosphate receptors (MPR). There are two MPR isoforms, the ~300-kDa cation-independent M6PRs/insulin-like growth factor-II (CI-MPR) and the 46-kD cation-dependent MPR (CD-MPR). Both MPRs bind to the mannose-6 phosphates (M6P) glycans on newly synthesized lysosomal enzymes or extracellular lysosomal enzymes for lysosomal delivery. The generation of a chimeric receptor consisting of the intracellular and transmembrane domains of the MPRs and replacing the extracellular ligand-binding domains with either ligand-binding domain for chemokins/cytokines, autoantigen peptide sequence for autoantibodies, single-chain variable fragment (scFV), or nanobodies targeting either chemokins/cytokines and extracellular therapeutic pathological proteins decreases the targeted extracellular therapeutic protein/autoantibody levels by binding at the cell surface and trafficking to the lysosome for degradation (FIGS. 1 and 2).

In various aspects, the LDCRs can be engineered to target proinflammatory molecules, including the Alzheimer's disease (AD) associated protein apolipoprotein E4 (ApoE4) or other proinflammatory proteins to target them for lysosomal degradation. Additional pathological proteins targeted include amyloid-β (Aβ) and tau protein in Alzheimer's disease (AD), α-synuclein in Parkinson's disease but are not limited to. Additional protein targets include, but are not limited to, autoantibodies in autoimmune diseases. By way of non-limiting examples, LDCRs can be engineered to target autoantibody epitopes such as the myelin basic protein that is targeted by autoantibodies in Multiple sclerosis. The autoantibodies in circulation will bind to their epitope (myelin-basic protein) on the LDCRs, leading to their trafficking to the lysosome for degradation.

In various aspects, the LDCRs can be engineered to utilize different receptors, including but not limited to the asialoglycoprotein receptor (ASGPR), which is specifically expressed in the hepatocyte cell membrane and binds, internalizes, and clears circulating extracellular glycoproteins containing galactose or N-acetylgalactosamine residues by lysosomal degradation. ASGPR is formed by a major subunit named ASGPR-1 (48 kDa) and a minor subunit named ASGPR-2 (40 kDa) that form homo or hetero-oligomers at the cell surface. ASGPR contains an intracellular domain, transmembrane domain, stalk region, and carbohydrate recognition domain. A chimeric receptor of the cytoplasmic and transmembrane domains of the ASGPR that are expressed in hepatocytes can increase the efficacy for clearing circulating autoantibodies by lysosomal trafficking and degradation. To engineer the new LDCR, the extracellular stalk region and carbohydrate recognition binding domain of the ASGPR receptor is exchanged the extracellular ligand-binding domains with either ligand-binding domain for chemokins/cytokines, autoantigen peptide sequence for autoantibodies, single-chain variable fragment (scFV), or nanobodies targeting either chemokins/cytokines and extracellular therapeutic pathological. proteins

In the present disclosure, compositions and methods have been devised to target extracellular proteins of interest to the lysosome for degradation.

Gene cell therapies can be used in this technology to incorporate LDCRs into cells.

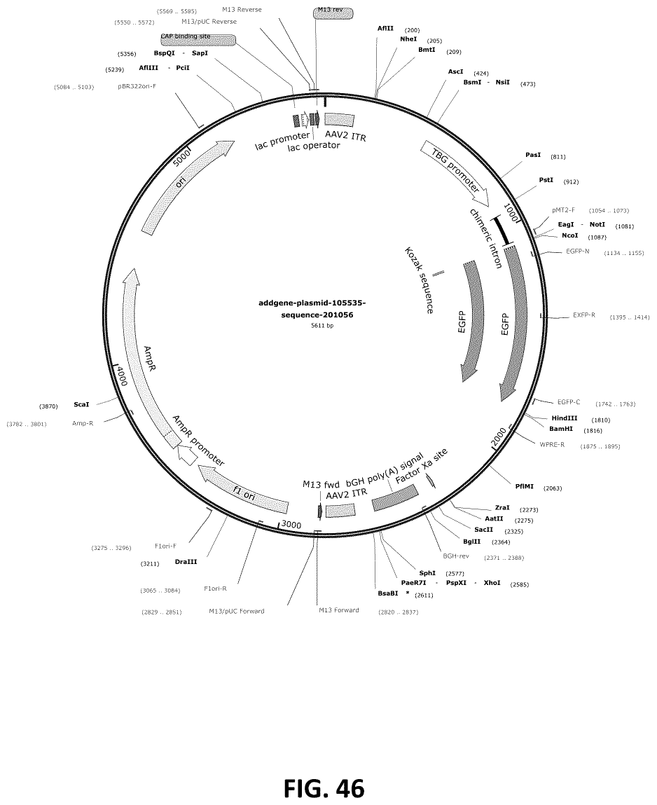

In some aspects, cells can be transduced with adeno-associated virus (AAV) expressing the LDCRs. By way of non-limiting example, an AAV plasmid map is provided at FIG. 46. In other aspects, chimeric antigen receptor T-cell therapy can be used to incorporate LDCRs into cells.

### Protein Degradation Agents

As described herein, protein expression has been implicated in various diseases, disorders, and conditions. As such, modulation of proteins (e.g., modulation of protein by degradation) can be used for the treatment of such conditions. A protein degradation agent can modulate protein response or induce or inhibit a protein. Protein degradation can comprise modulating the expression of proteins on cells, modulating the quantity of cells that express a protein, modulating the quality of the protein-expressing cells or modulating the quantity of a protein secreted into the extracellular matrix.

Protein degradation agents can be any composition or method that can modulate protein expression on cells (e.g., by targeted degradation). For example, a protein degradation agent can be an activator, an inhibitor, an agonist, or an antagonist. As another example, protein degradation can be the result of gene editing.

A protein degradation agent can target a protein to be degraded by the lysosome. A protein degradation agent can be a lysosomal degrading chimeric receptor (LDCR).

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41 (fraction G/C content)−0.63(% formamide)−(600/l). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a disease or disorder in a subject in need of administration of a therapeutically effective amount of a LDCR, so as to reduce expression of a disease associated protein.

Methods described herein are generally performed on a subject in need thereof. A subject in need of the therapeutic methods described herein can be a subject having, diagnosed with, suspected of having, or at risk for developing an immune-mediated inflammatory diseases, autoimmune diseases, Alzheimer's Disease, or other diseases. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a LDCR is, for example, an amount that would cause the desired therapeutic effect in a subject while minimizing undesired side effects. In various embodiments, an effective amount of a LDCR described herein can substantially reduce expression of a protein, slow the progress of a disease, or limit the development of a disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a LDCR can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a disease.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an LDCR can occur as a single event or over a time course of treatment. For example, a LDCR can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease.

A LDCR can be administered simultaneously or sequentially with another agent, such as an antibiotic, an anti-inflammatory, or another agent. For example, an LDCR can be administered simultaneously with another agent, such as an antibiotic or an anti-inflammatory. Simultaneous administration can occur through the administration of separate compositions, each containing one or more of a LDCR, an antibiotic, an anti-inflammatory, or another agent. Simultaneous administration can occur through the administration of one composition containing two or more of a LDCR, an antibiotic, an anti-inflammatory, or another agent. A LDCR can be administered sequentially with an antibiotic, an anti-inflammatory, or another agent. For example, an LDCR can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, rectal, AAV-gene expression, mRNA vaccine-like approaches and CAR-T like approaches.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a LDCR, solubilizers, sterile packaging, and any combination thereof. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Lysosomal Degrading Chimeric Receptors (LDCRs)

To develop a targeted protein degradation approach that reduces proinflammatory proteins, including but not limited to APOE4 and TNFα levels in the central nervous system (CNS), enhancing the therapeutic potential in AD, or peripherally proinflammatory proteins such as cytokines/chemokines, enhancing the therapeutic potential of immune-mediated inflammatory disease, or autoantibodies, enhancing the therapeutic potential in autoantibody diseases the following experiments were conducted. A lysosomal degrading chimeric receptor (LDCR) was generated to target extracellular and secretory proteins for degradation utilizing the mannose-6-phosphate receptor (MPR) or the asialoglycoprotein receptor (ASGPR).

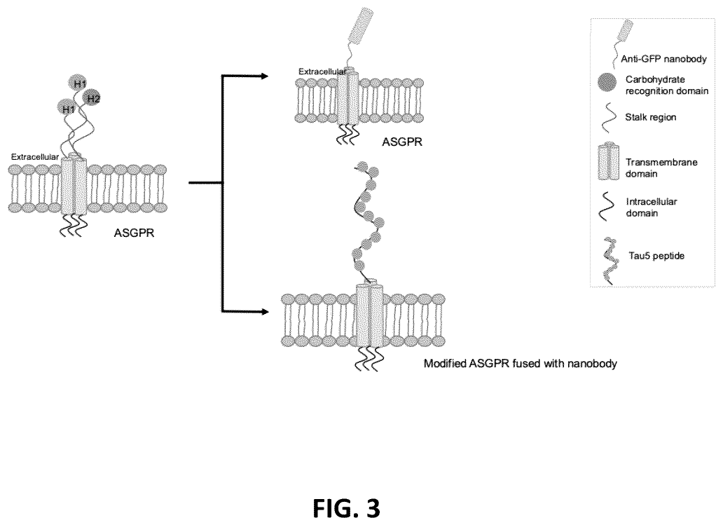

Targeted protein degradation (TPO) via proteasome and lysosome provided new tools for therapeutic approaches which can selectively target disease-causing protein for degradation. Among recent approaches for TPO, we have developed an approach termed lysosomal degrading chimeric receptors (LDCRs), which target extracellular proteins including autoantibodies of interest to lysosome for degradation. This mechanism for targeting extracellular proteins or autoantibodies for degradation involved the reengineering of the mannose 6-phosphate receptors (MPR). There are two MPR isoforms, the ~300-kDa cation-independent M6PRs/insulin-like growth factor-II (CI-MPR) and the 46-kD cation-dependent MPR (CD-MPR). Both MPRs bind to the mannose-6 phosphates (M6P) glycans on newly synthesized lysosomal enzymes or extracellular lysosomal enzymes for lysosomal delivery. We hypothesized generating a chimeric receptor consisting of the intracellular and transmembrane domains of the MPRs and replacing the extracellular ligand-binding domains with a nanobody targeting a protein of interest or an autoantibody epitope sequence would decrease the targeted extracellular protein/autoantibody levels by binding at the cell surface and trafficking to the lysosome for degradation (FIG. 1, FIG. 2, FIG. 3).

To validate this technology, we replaced the extracellular ligand binding domains of the MPRs with an anti-GFP nanobody and demonstrated the anti-GFP LDCRs bind extracellular GFP and traffic to the lysosome for degradation. The attached grant application provides preliminary data and details on the anti-GFP LDCRs. The two goals include developing LDCRs that target proinflammatory molecules, including the Alzheimer's disease (AD) associated protein apolipoprotein E4 (ApoE4), by replacing the anti-GFP on our LDCRs with nanobodies targeting ApoE4 or other proinflammatory proteins to target them for lysosomal degradation. This approach will open new therapeutic strategies for AD and other neurodegenerative diseases that can be delivered into the brain by adeno-associated virus gene therapy.

In addition to targeting proinflammatory proteins, we also target autoantibodies in autoimmune diseases. Autoimmune diseases consist of over 80 disorders in which the immune system recognizes self-tissues/cells as non-self and produces autoantibodies that attack the tissues/cells, causing organ dysfunction and inflammation. Current therapies for autoimmune diseases include anti-inflammatory strategies, and currently, no strategy has been developed to eliminate the autoantibody. Therefore, developing therapeutic approaches targeting the autoantibody is of crucial importance. To target autoantibodies, we will utilize our LDCR technology and replace the anti-GFP nanobody with an autoantibody epitope such as the myelin basic protein that is targeted by autoantibodies in Multiple sclerosis. The autoantibodies in circulation will bind to their epitope (myelin-basic protein) on our LDCRs, leading to their trafficking to the lysosome for degradation. We have developed LDCRs containing the epitope for the anti-tau-5 monoclonal antibody (mAB) specific for human tau protein. The anti-tau-5 mAB epitope has been mapped to amino acids 160-167, and generated into a peptide seq spanning the epitope region (amino acids 154-174) of human tau. Utilizing this epitope region, we generated a chimeric tau-epitope by replacing the anti-GFP nanobody sequence in the LDCRs. This can be utilized as AAV gene therapy and can express the LDCRs on hepatocytes for delivery.

We have also generated a second generation of LDCRs utilizing the asialoglycoprotein receptor (ASGPR), which is specifically expressed in the hepatocyte cell membrane and binds, internalizes, and clears circulating extracellular glycoproteins containing galactose or N-acetylgalactosamine residues by lysosomal degradation. ASGPR is formed by a major subunit named ASGPR-1 (48 kDa) and a minor subunit named ASGPR-2 (40 kDa) that form homo or hetero-oligomers at the cell surface. ASGPR contains an intracellular domain, transmembrane domain, stalk region, and carbohydrate recognition domain. A chimeric receptor of the cytoplasmic and transmembrane domains of the ASGPR are expressed in hepatocytes may increase the efficacy for clearing circulating autoantibodies by lysosomal trafficking and degradation. To engineer the new LDCR, we exchanged the extracellular stalk region and carbohydrate recognition binding domain of the ASGPR receptor, with the anti-tau5 mAB epitope region (amino acids 154-174 of human tau).

### Methods

We evaluated this series of LDCRs both in vitro and in vivo models to determine the LDRC that is most proficient at clearing extracellular anti-tau5 mAB by lysosomal degradation.

To evaluate the efficacy of our modified LDCR-MPRs derived and LDCR ASGPR derived to bind extracellular tau5 antibody and traffic to the lysosome for degradation.

Human hepatocyte cell lines (Huh7, HepG2) that have the capacity to catabolize large amounts of proteins without toxicity and can be used to express the various LDCRs targeting the anti-tau5 mAB. To track extracellular anti-tau5 mAB binding to the receptor and localization into the lysosome of hepatocytes, we will label the anti-tau5 mAB with pHrodo-red dye, a pH-sensitive dye that fluoresces under acidic conditions including the lysosome. For analyses, we perform live imaging and flow cytometry to measure anti-tau5 mAB uptake from the condition media and lysosomal localization with lysotracker. Additional analyses will include immunohistochemistry with anti-IgG mouse targeting anti-tau5 mAB, lysosomal marker LAMP1, and anti-HA for the LDCRs.

To determine whether the degradation of the anti-tau5 mAB is lysosomal dependent, we block lysosome activity with bafilomycin and measure the accumulation of anti-tau5-pHrodo-red mAB with flow cytometry and by immunofluorescent microscopy with anti-IgG mouse.

To evaluate the clearance of extracellular anti-tau5 mAB following the expression of the various LDCRs in hepatocytes, we collect condition media and cell lysate time-dependently and quantify anti-tau5 mAB by immunoblots and ELISA-based measurements.

### Mouse Model to Evaluate the Various LDCRs Targeting Circulating Anti-Tau5 mAB for Lysosomal Degradation In Vivo:

We express the various LDCRs in hepatocytes by intravenous injection (IV) of the AAV 2/8 serotype expressing each LDCR under a hepatocyte-specific promotor.

After 30 days of AAV2/8 expression, an IV of anti-tau5-pHrodo-red mAB is administered and the serum collected weekly over a month. We measure the anti-tau5 mAB concentration with ELISA-based measurements and flow cytometry. Additional analyses include measuring liver enzymes to evaluate potential toxicity following AAV expression of the various LDCRs and clearance of the anti-tau5 mAB. We also isolate the liver and other tissues, including the spleen and kidney, for histological analysis to evaluate potential toxicity.

Evaluation of the Experimental Autoimmune Encephalomyelitis (EAE) mouse model of Multiple Sclerosis is conducted utilizing an LDCR fused to the myelin basic protein (MBP). The EAE model is induced by immunizing mice against myelin-derived antigens, which leads to the production of anti-myelin autoantibodies and axonal degeneration. We hypothesize expressing an LDCR fused to MBP will target autoantibodies targeting myelin in circulation for lysosomal degradation.

### Example 2—Engineering a LDCR that Degrades Apoe4 to Suppress Neuroinflammation in Ad

The APOE4 (E4) gene is the most potent genetic risk factor for developing late-onset Alzheimer's disease (AD) and is expressed in more than half of AD patients. The pathophysiological mechanism by which E4 influences AD progression remains entirely unclear. However, evidence supports that E4 influences Aβ plaque deposition. In addition to the E4 role in AD, emerging evidence suggests E4 progresses other neurodegenerative diseases independent of Aβ plaques. We previously demonstrated that E4 influences tau pathology by exacerbating an inflammatory response independent f Aβ plaques. We have also shown that APOE influences Parkinson's disease (PD) progression associated with α-synuclein mutations by knockout of the murine APOE, which increased survival of transgenic mice expressing mutant human α-synuclein. More recent studies have further substantiated that the E4 gene increases α-synuclein pathology in human α-synuclein transgenic mice, and PD patients with two E4 alleles displayed the fastest disease progression. These studies highlight E4 as a therapeutic target for other neurodegenerative diseases. In line with this notion, we evaluated passive immunotherapy targeting E4 in APP/PS1 mice, which decreased Aβ plaques but was limited to E4 bound to Aβ plaques. The protective effects of passive immunotherapy targeting E4 further depended on glial Fcγ receptors that mediated the uptake of E4 associated with Aβ plaques. Although these studies demonstrate that anti-E4 immunotherapies decrease plaque burden, the necessity for E4 to be associated with plaque makes it inefficient for other degenerative diseases. Moreover, Fcγ mediated glial uptake may lead to adverse effects by a pro-inflammatory response. In addition, a previous study demonstrated that lowering extracellular E4 protein by overexpressing LDLR reduced tau-related pathologies in tau-tg mice. However, the LDLR binds other ligands and is not exclusively an E4 receptor. Therefore, we aim to develop an immunotherapeutic approach that reduces E4 levels in the CNS, enhancing the therapeutic potential in AD that could be expanded to other neurodegenerative diseases.

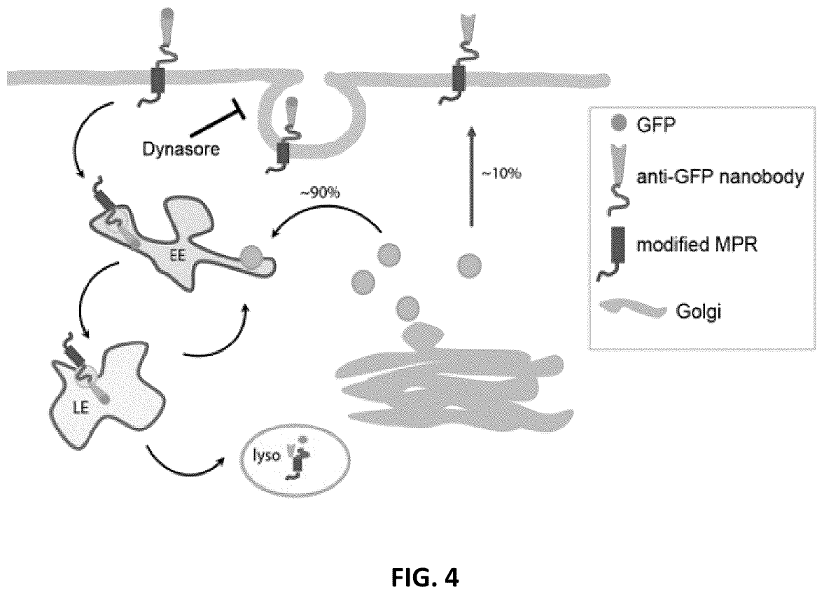

To achieve this goal, we designed a lysosomal degrading chimeric receptor (LDCR) that targets extracellular and secretory proteins for degradation. We utilized the mannose-6-phosphate receptor (MPR) to generate the LDCR. There are two MPR isoforms, the ~300-kDa cation-independent MPR/insulin-like growth factor-II (CI-MPR) and the 46-kD cation-dependent MPR (CD-MPR). The vast majority of MPRs recycle between the trans-Golgi, early/late endosomes, and lysosomes and bind to the mannose-6 phosphates (M6P) glycans on newly synthesized lysosomal enzymes at the trans-Golgi network for lysosomal delivery. In addition, a fraction of the MPRs traffic to the cell surface to bind and internalize extracellular lysosomal enzymes containing M6P and have been utilized for enzyme replacement therapies for lysosomal storage diseases. Therapeutic strategies have also recently been developed using the M6P ligand, including the lysosome-targeting chimeras (LYTACs), to degrade extracellular proteins. The LYTAC technology combined a synthetic nonhydrolyzable M6P glycopeptide with antibodies that bind to targeted antigens. The conjugated M6P glycopeptide on antibodies acts as a ligand for the MPRs. The MPRs mediate the endocytosis of the antibody-bound antigen trafficked to the lysosome for degradation. While this technology provides a method for targeting extracellular proteins for lysosomal degradation, it is still limited by the need to conjugate M6P glycopeptide onto antibodies and the limitations of passive immunotherapies previously discussed. We hypothesized that a chimeric receptor consisting of the transmembrane-cytoplasmic domains of the MPRs fused to a nanobody targeting a secreted protein would decrease the extracellular protein levels by binding at the cell surface and trafficking to the lysosome for degradation. As a proof of concept, we fused a nanobody targeting GFP to the transmembrane-cytoplasmic domains of the MPRs (CD-MPR and CI-MPR). We demonstrate that extracellular GFP protein is endocytosed and trafficked to the lysosome for degradation following the expression of the LDCR (FIG. 4).

### Results

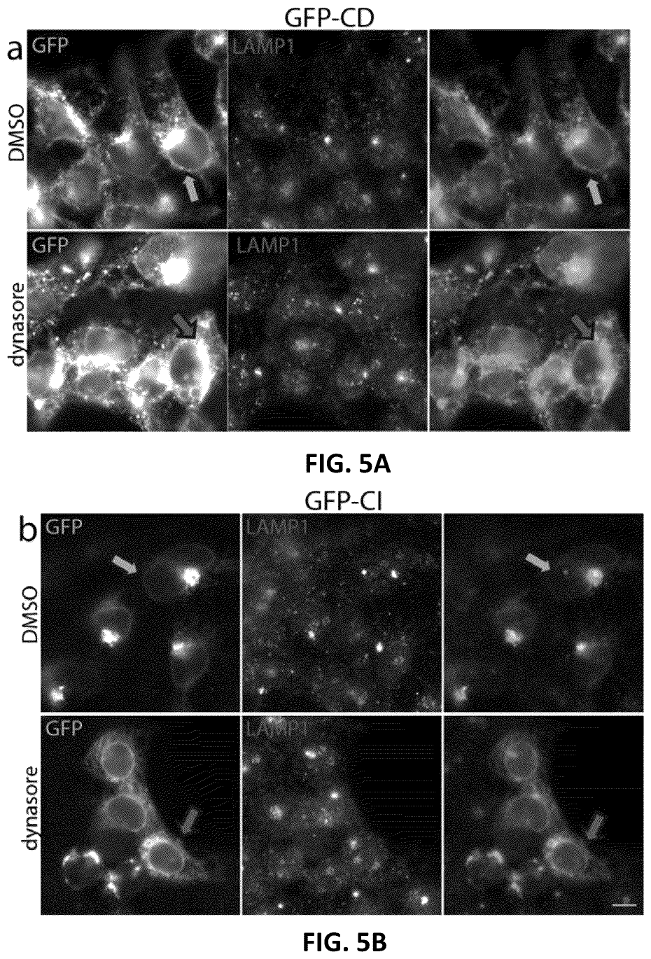

Although the MPRs have been shown to internalize from the cell surface constitutively, previous studies suggested the inactivation of the ligand binding domains decreases the internalization rate and may also lead to accumulation within the Golgi. Therefore, we first investigated whether removing the MPRs ligand-binding domains alters lysosomal localization by designing a chimeric receptor with GFP. The chimeric receptor consisted of the endogenous N-terminal signal sequence, the transmembrane, and cytoplasmic domains of the CD-MPR and CI-MPR, replacing the ligand binding domains with GFP protein (GFP-CD and GFP-CI). This approach lets us visualize and traffic the chimeric receptor in real time. Next, we transfected HEK293t cells, which are amenable for transfection, and evaluated protein trafficking of our GFP-chimeric MPRs to determine whether the ligand binding domain is necessary for lysosomal localization. Immunofluorescence (IF) analyses with LAMP1 and GFP demonstrated the co-localization of both receptors in the lysosome (FIG. 5A, FIG. 5B). We next determined whether the ligand binding domain was necessary for cell surface trafficking and treated the cells with Dynasore, a dynamin inhibitor. Dynamin inhibition blocks receptor internalization, which leads to the accumulation of the GFP chimeric-MRPs at the cell surface (FIG. 4, FIG. 5A, FIG. 5B). In control experiments, expression of an empty vector did not display any GFP binding to the HEK293t cells.

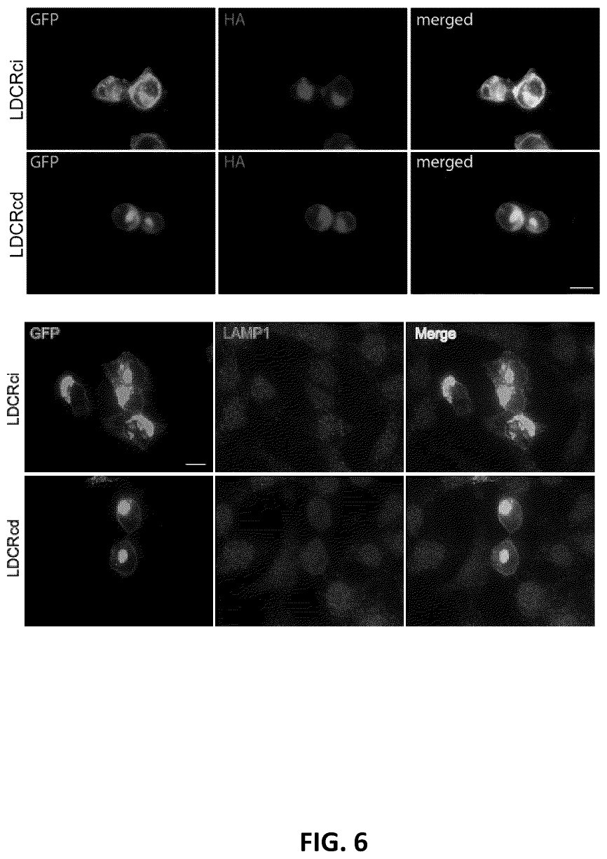

This analysis suggested that the ligand binding domain is unnecessary for proper receptor trafficking and lysosomal localization. Based on this analysis, we reasoned the ligand binding domain could be replaced with an anti-single chain variable fragment or nanobodies targeting candidate extracellular protein for delivery into the lysosomal for degradation. Next, we set out to engineer our LDCR by replacing the ligand binding domains of the MPRs with an anti-GFP nanobody for a proof of concept. The anti-GFP nanobody was previously generated from a phagemid library of antibodies of GFP-immunized camelids. The LDCR consisted of the transmembrane and cytoplasmic domains containing an HA-tag of the CD-MPR and CI-MPR, replacing the ligand binding domains with anti-GFP nanobody and the endogenous secretory signal N-terminal sequence (LDCRci and LDCRcd) (FIG. 6). We first evaluated the ability of the anti-GFP nanobody fused to the transmembrane-cytosolic domains of the MPRs to bind extracellular GFP protein following the transfection of HEK293t cells and adding GFP protein (0.4 ug/ml) to the medium. IF analysis revealed the LDCRci and LDCRcd (anti-HA) colocalized with GFP protein, indicating binding to extracellular GFP protein (FIG. 6).

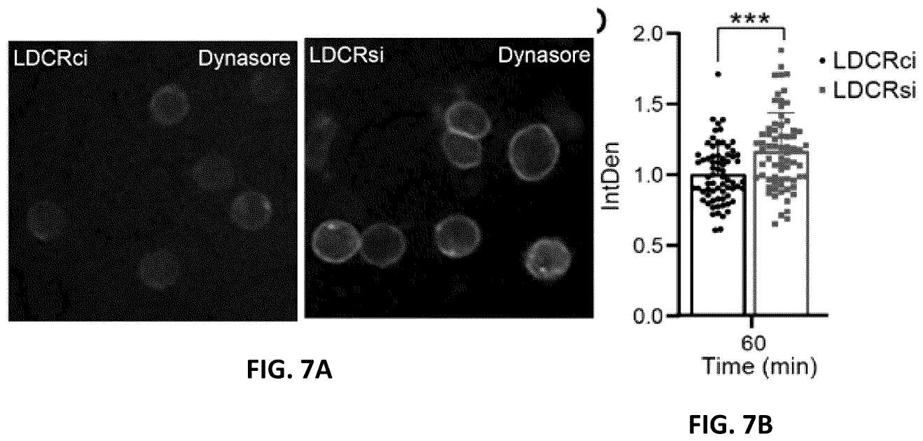

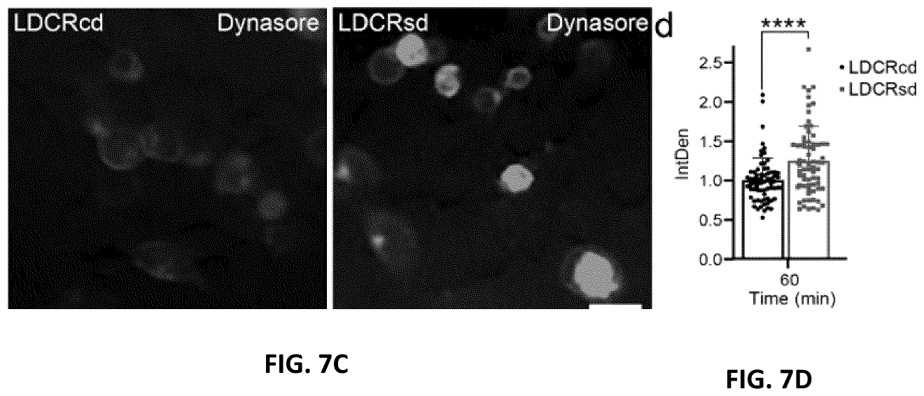

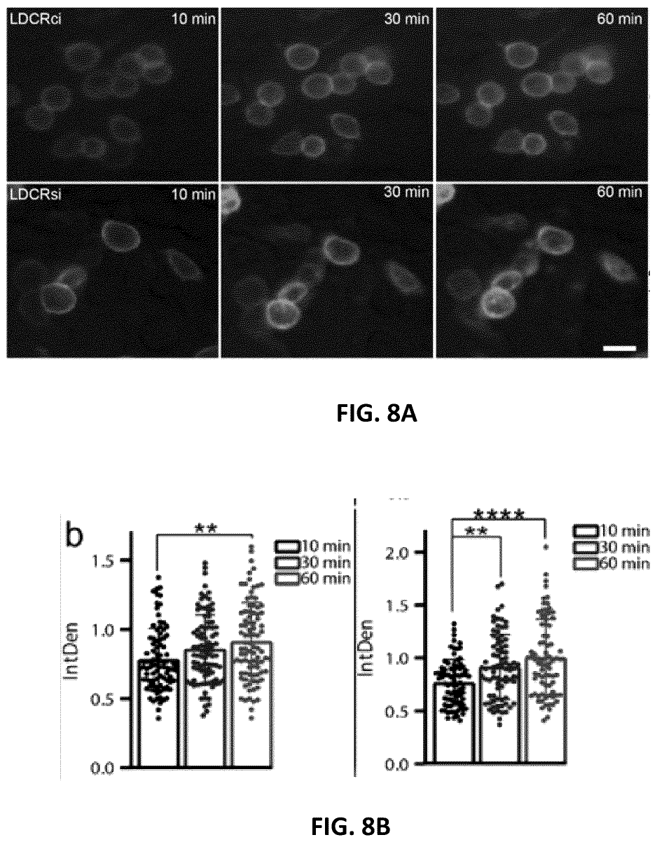

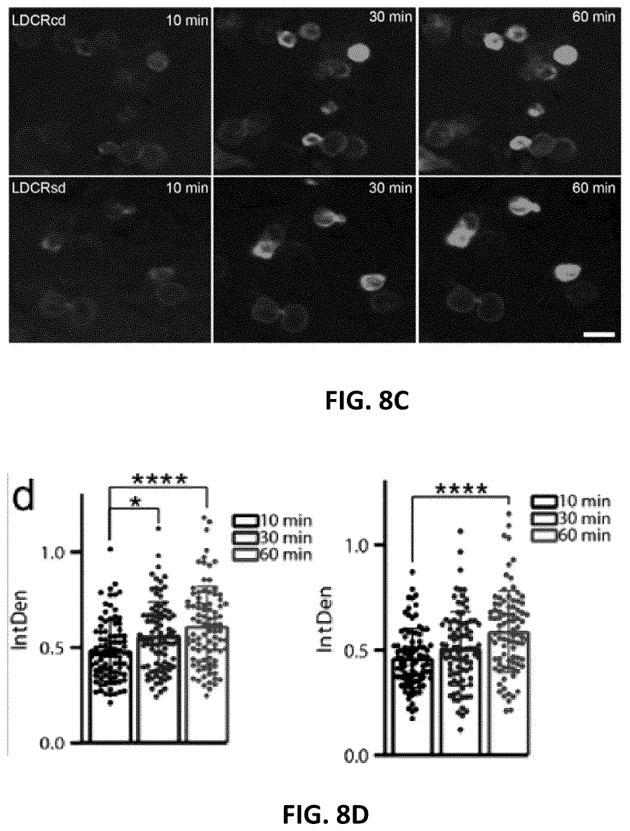

In addition to targeting extracellular proteins for lysosomal localization, the MPRs bind newly synthesized lysosomal enzymes at the trans-Golgi network for lysosomal delivery. Thus, the LDCR may also potentially bind newly synthesized candidate secretory proteins at the trans-Golgi for lysosomal delivery, further decreasing candidate protein levels intracellularly. However, the trans-Golgi pH ~6.0 is not optimal for nanobodies or scFvs binding to ligands (~7.4 pH) and will depend highly on their affinity. To potentially overcome this limitation, we hypothesized that increasing cell membrane localization would improve the targeting of extracellular proteins for lysosomal degradation (FIG. 4). The signal sequence of the N-terminal directs the insertion of the receptor into the membrane of the ER for insertion into the membrane. Previous studies have illustrated that the N-terminal sequences are diverse and vary in efficiency for membrane targeting and secretory pathways. Therefore, we hypothesized switching out the endogenous MPRs N-terminal sequences for an N-terminal sequence utilized by the secretory pathway would increase the cell membrane trafficking of the LDCRs. To test this hypothesis, we replaced the endogenous N-terminal sequence with an N-terminal sequence that we previously used to secrete full-length two-chained antibody or a scFV referred to as LDCRsi and LDCRsd. Next, we determined if the LDCRsi and LDCRsd increased cell surface localization and treated HEK293t cells expressing the four LDCRs with Dynasore (60 min) to block dynamin-mediated endocytosis. Following Dynasore treatment, we evaluate the amount of GFP protein bound to the cell surface. This analysis illustrated blocking dynamin-mediated endocytosis increased the accumulation of the LDCRsi and LDCRsd, leading to more GFP protein at the cell surface relative to the LDCRci and LDCRcd containing the endogenous N-terminal sequence (FIG. 7A, FIG. 7B). In addition, several cells expressing the LDCRsd display a robust accumulation of GFP potentially intracellularly despite Dynasore treatment (FIG. 7C, FIG. 7D). This initial analysis suggested the modified N-terminal sequence increased cell surface trafficking. We next evaluated the four LDCRs by live imaging analysis by treating the condition media with GFP protein (0.4 ug/ml) and imaging over 60 minutes. This analysis demonstrated that all four LDCRs, with and without the secretory signal peptide bound to extracellular GFP protein, increased time-dependently (FIG. 8A, FIG. 8B, FIG. 8C, FIG. 8D). The expression of an empty vector did not display any GFP binding.

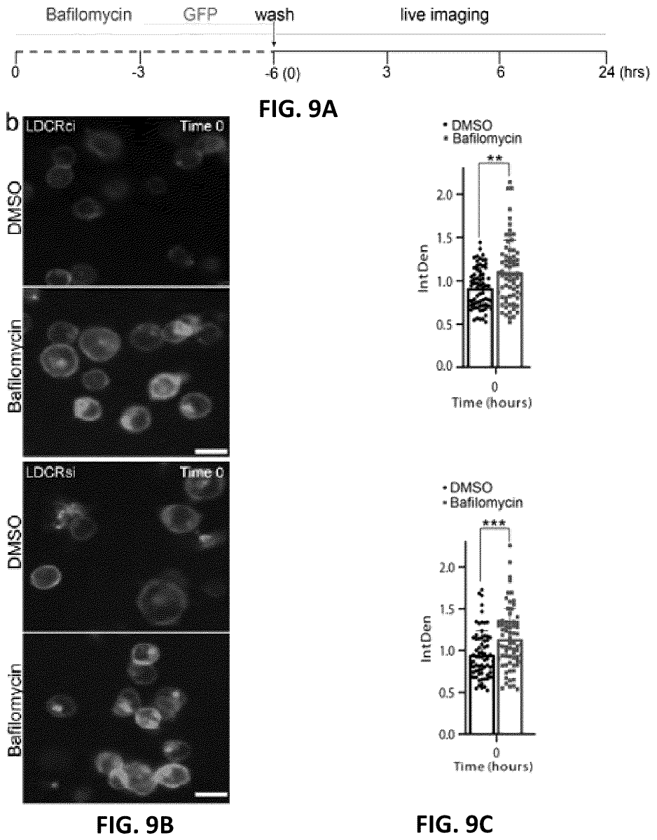

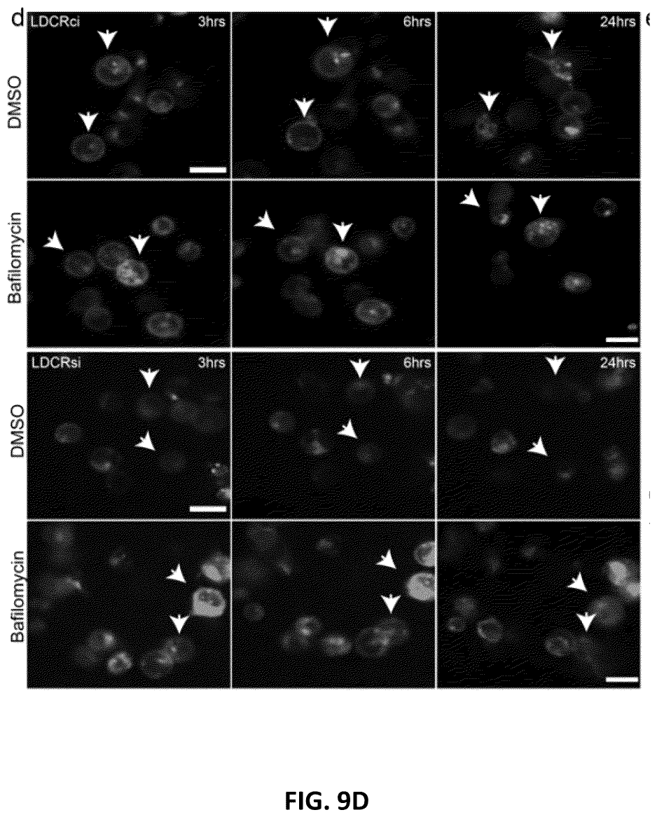

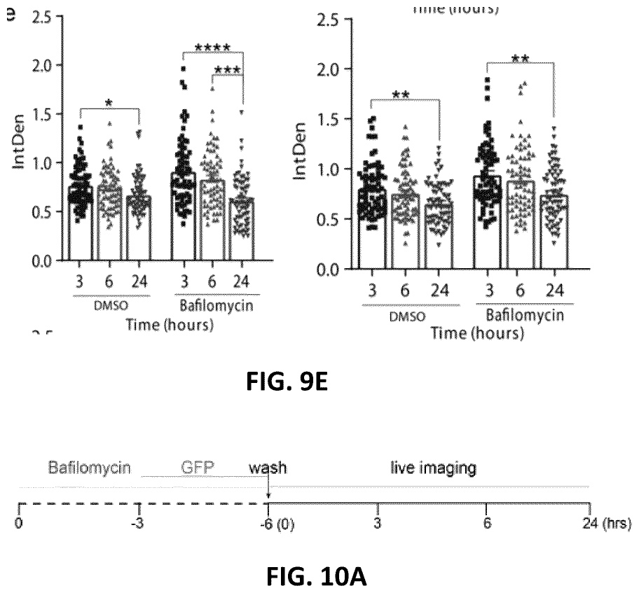

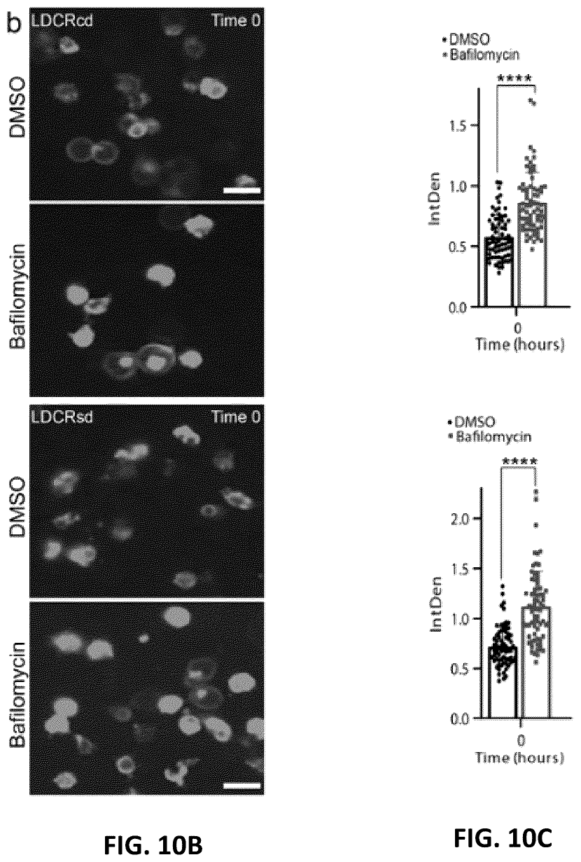

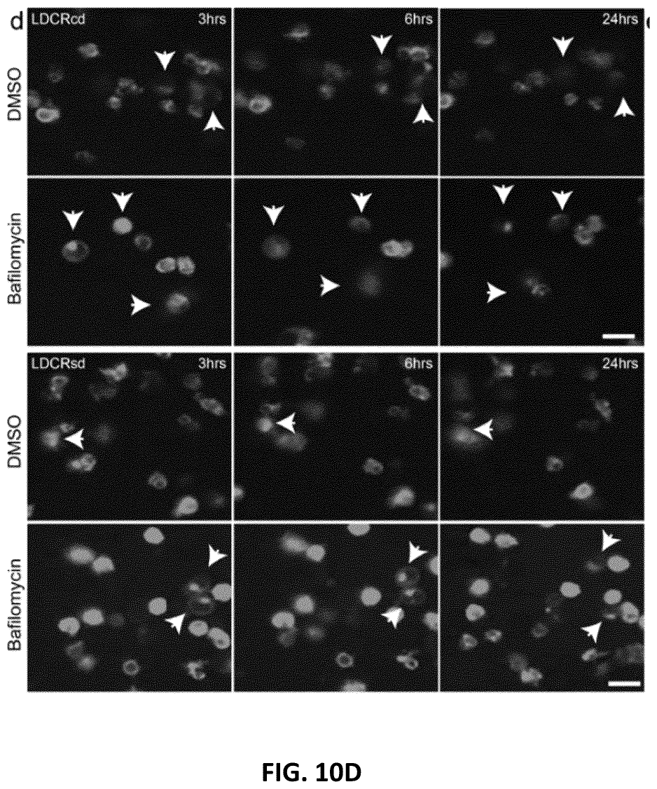

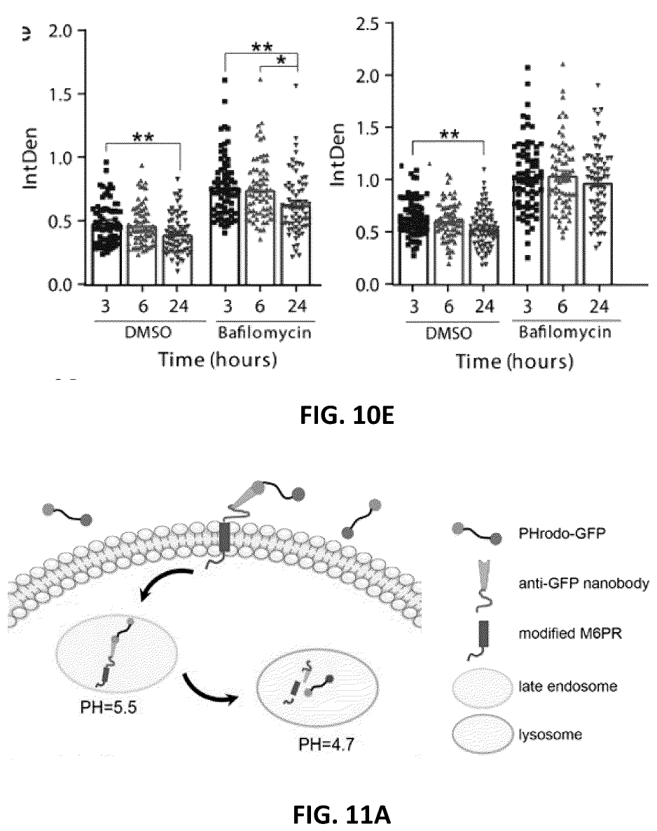

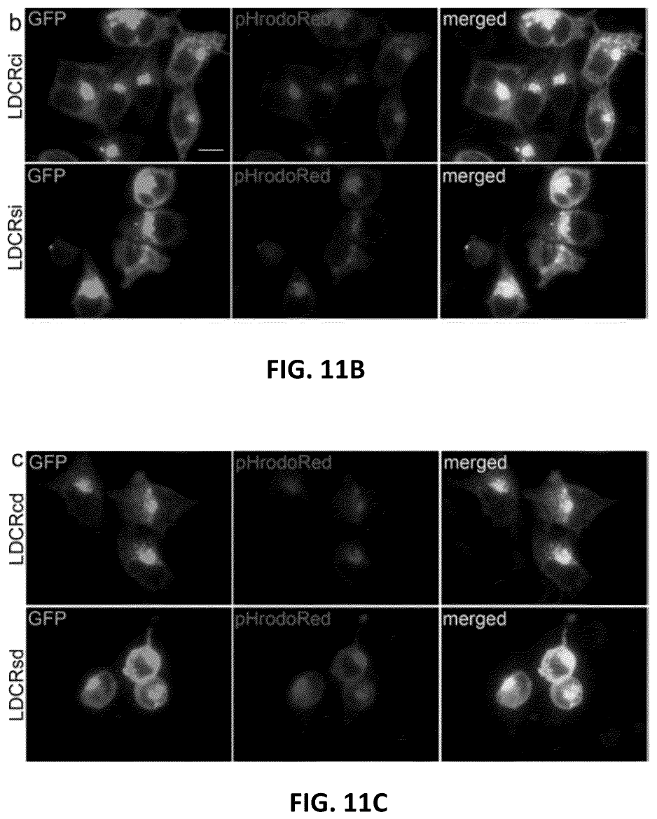

To determine if the four LDCRs traffic extracellular GFP to the lysosome, we treated HEK293t cells expressing four LDCRs with bafilomycin to block lysosomal degradation or control DMSO (3 hr), followed by treating the condition media with GFP protein (3 hrs). Next, we analyzed the GFP fluorescence intensity, demonstrating that lysosomal inhibition increased the accumulation of intracellular GFP protein relative to control DMSO for all four LDCRs (FIG. 9A, FIG. 9B, FIG. 9C, FIG. 10A, FIG. 10B, FIG. 10C). We next evaluated whether washing out bafilomycin and GFP from the condition media would degrade GFP protein over time and decrease GFP intensity. This analysis demonstrated a time-dependent decrease in GFP intensity for all four LDCRs following removing the lysosomal inhibitor bafilomycin, suggesting lysosomal-mediated degradation (FIG. 9D, FIG. 9E, FIG. 10D, FIG. 10E). To further evaluate LDCRs trafficking, we conjugated GFP protein (489/508 nm) with pHrodo-Red (ThermoFisher P36600), a fluorescent dye (560/580 m) that increases intensity as pH decreases utilized for analyzing endocytosis and lysosomal delivery. HEK293t cells expressing the four LDCRs were treated with GFP-pHrodoRed in the condition media, followed by IF analysis revealing lysosomal localization of GFP pHrodoRed for all four LDCRs (FIG. 11A, FIG. 11B, FIG. 11C).

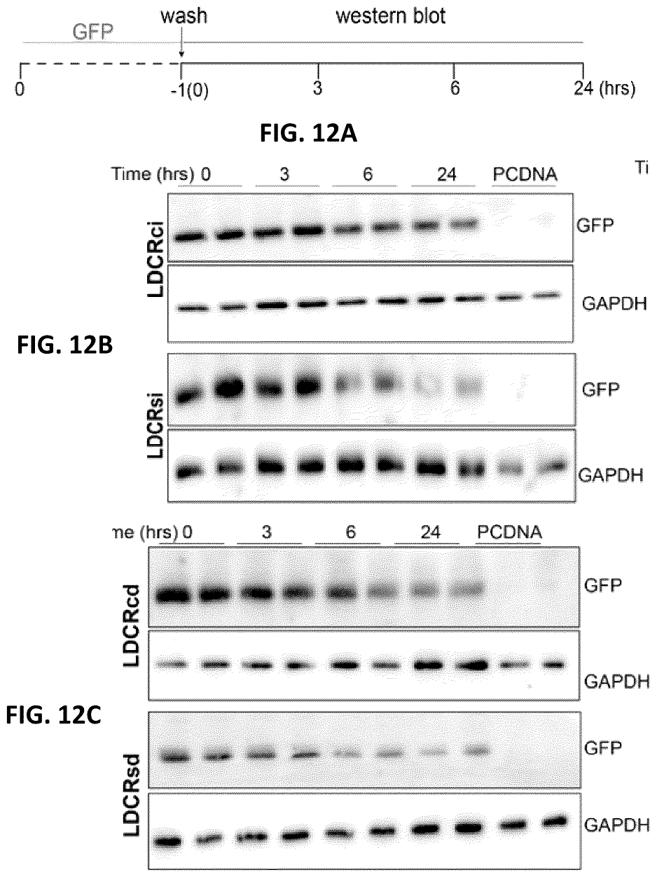

We next evaluated GFP protein levels in cell lysates of HEK293t cells expressing the four LDCRs by immunoblotting. HEK293t cells expressing the four LDCRs were treated with GFP protein in condition media for 60 min, followed by washing and collecting at time 0, 3 hr, 6 hr, and 24 hr post-GFP treatment. Immunoblotting analysis demonstrated a time-dependent decrease in the GFP protein lysates following the expression of each of the four LDCRs (FIG. 12A, FIG. 12B, FIG. 12C, FIG. 14).

### Characterize LDCRs Trafficking and Enhance its Degradation Capacity by Increasing Cell Surface Stability.

While we provide substantial preliminary data for the LDCR binding extracellular proteins and trafficking to the lysosome for degradation, additional experiments are needed to rigorously evaluate their mechanism for degradation and determine the most effective LDCR in reducing extracellular proteins without adverse effects. For these proposed studies we will generate stably expressing HEK293t cell lines for each of the LDCRs to reduce variability and develop control LDCRs lacking the anti-GFP nanobody for a negative control. In addition, we hypothesized that replacing the endogenous N-terminal sequence for the MPRs with an N-terminal sequence for the secretory pathway would increase cell surface localization of the LDCRs and increase the binding and uptake of extracellular proteins. To build from our preliminary data that analyzed the LDCRs following Dynasore treatment, we will measure the cell surface levels of the four LDCRs by Cell Surface Protein Biotinylation and Isolation assay (Pierce A44390). Following 48 hours of post-transfection of the four LDCRs, we will biotinylate and isolate surface proteins according to the manufacturer's protocol and analyze the LDCRs protein levels by immunoblotting analysis. In addition, we will analyze the LDCRs surface levels following the inhibition of dynamin-mediated endocytosis with Dynasore treatment. To identify the LDCR that is most effective in endocytosis and degradation of extracellular proteins, we aim to analyze and compare the four LDCRs by flow cytometry. HEK293t cells expressing the four LDCRs will be treated with GFP-pHrodoRed in the condition media and collected for flow cytometry measurements at 5 min, 10 min, 20 min, 40 min and 60 min. In controlled experiments, HEK293t cells expressing the four LDCRs will be treated with Dynasore to block dynamin-mediated endocytosis, decreasing GFP-pHrodoRed uptake. In addition, HEK293t cells will be treated with bafilomycin to block lysosomal degradation, increasing the GFP signal intracellularly and neutralizing the pHrodoRed signal. To identify the LDCR most proficient in endocytosis degrading extracellular GFP protein, a ratio of GFP to pHrodoRed will be analyzed at each time point utilizing the integrated fluorescence intensity.

In additional preliminary data, we evaluated the GFP protein levels in HEK293t lysates, demonstrating a time-dependent decrease following the expression of the four LDCRs. To build on these preliminary data, we aim to analyze HEK293t lysates expressing the LDCRs following GFP treatment and lysosomal inhibition with bafilomycin or control DMSO to determine if the decrease in GFP protein levels depends on lysosomal degradation. Immunoblotting analysis and ELISA-based measurements will analyze GFP protein levels from lysates (GFP ELISA kit Abcam ab171581). We will also analyze the GFP protein levels in the conditioned medium to determine the LDCR that is most effective in depleting extracellular GFP protein. Following 48 hours of post-transfection of the four LDCRs, we will treat the condition media with GFP protein (0.4 ug/ml) and collect the medium at times 0, 3 hr, 6 hrs, 12 hrs, and 24 hrs and determine GFP protein levels by ELISA analysis. To further evaluate GFP trafficking following endocytosis, we will analyze IF staining with early endosomal antigen (EEA1), late endosomal marker RAB7, Golgi marker GM130, and lysosomal marker LAMP1.

We will also analyze primary neurons and astrocytes to determine the efficiency of targeting extracellular proteins for lysosomal degradation and potential adverse effects with the four LDCRs. Primary neurons and astrocytes will be transduced with AAVs expressing the LDCRs, and GFP trafficking, and degradation will be analyzed utilizing the paradigms of live imaging, flow cytometry, biochemical, and IF analyses. Additional analyses for primary culture will include a cell viability assay (Promega Cell titer-Glo G9241) to determine the long-term expression of the LDCRs and the constitutive endocytosis and degradation of extracellular GFP. Primary neurons will be transduced with lentivirus encoding the LDCRs and controls at DIV 4. At the same time, primary astrocytes will be split and transduced with lentivirus encoding the LDCRs at DIV2. After 72 hours post-transduction, we will treat the condition media with GFP protein (0.4 ug/ml), and primary neurons or astrocytes will be collected for cell viability at DIV10. Primary astrocytes will be evaluated with a panel of proinflammatory genes by qPCR to evaluate if the constitutive endocytosis and degradation of extracellular GFP alters the astrocytic inflammatory state. Neuronal will further be evaluated for dendritic (MAP2A) and axonal abnormalities with IF analysis. Additional IF analysis for GFP trafficking will include EEA1, RAB7, GM130, and LAMP1.

### Characterization of Single-Domain Antibodies Targeting Human Lipidated ApoE Protein.

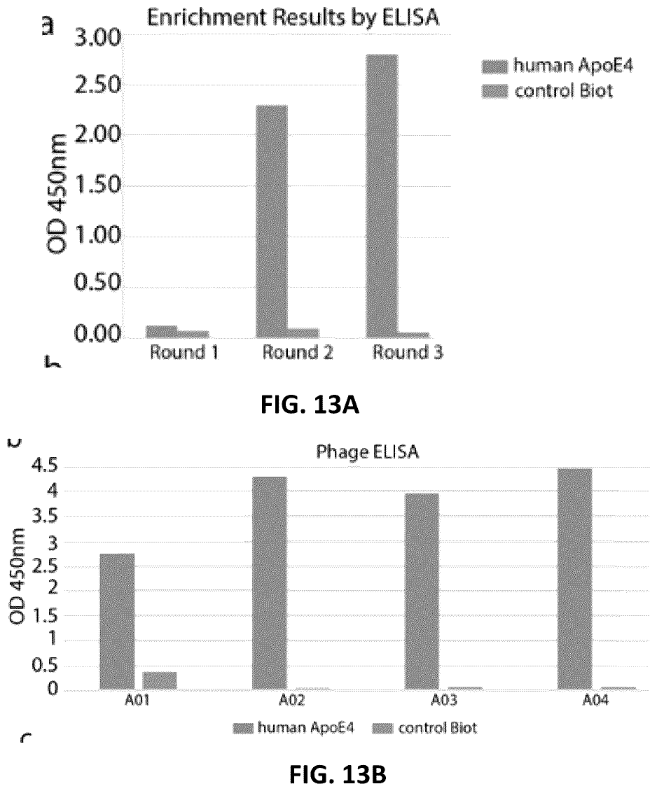

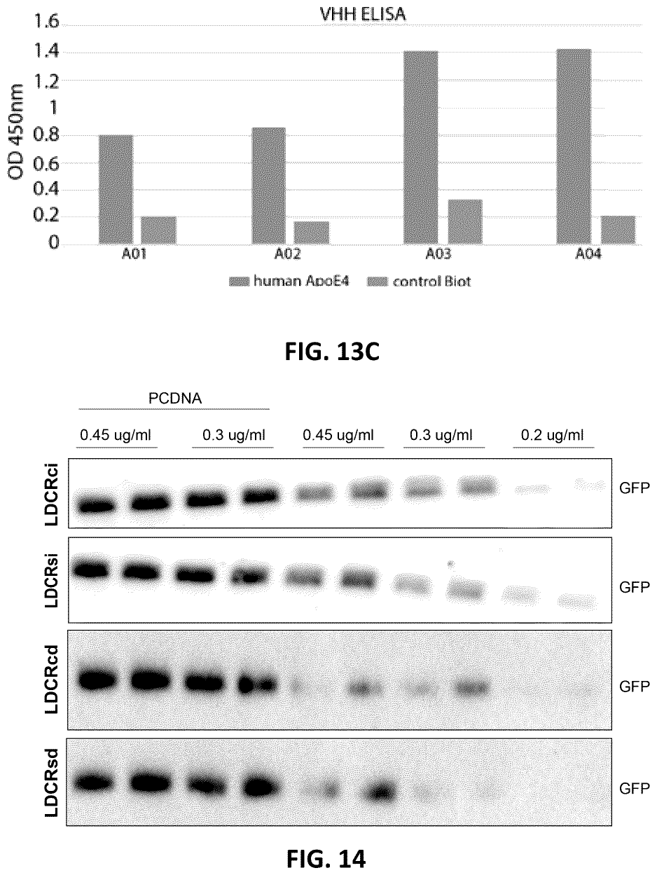

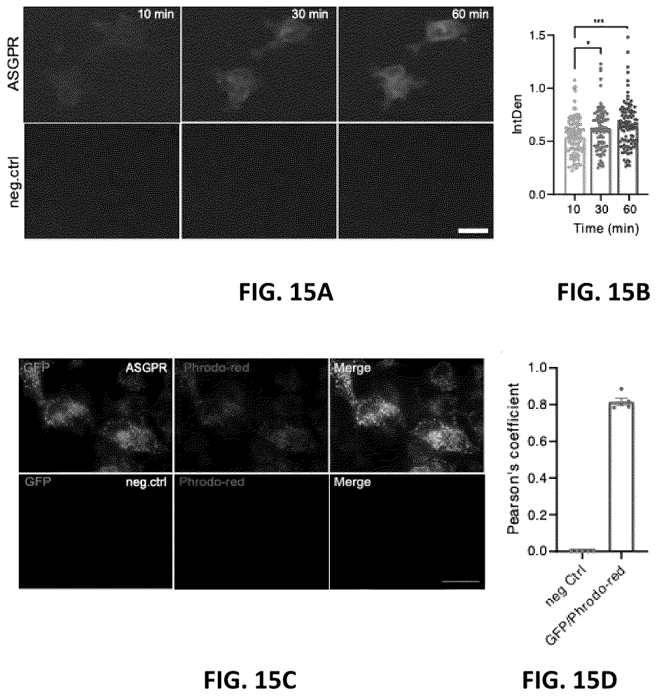

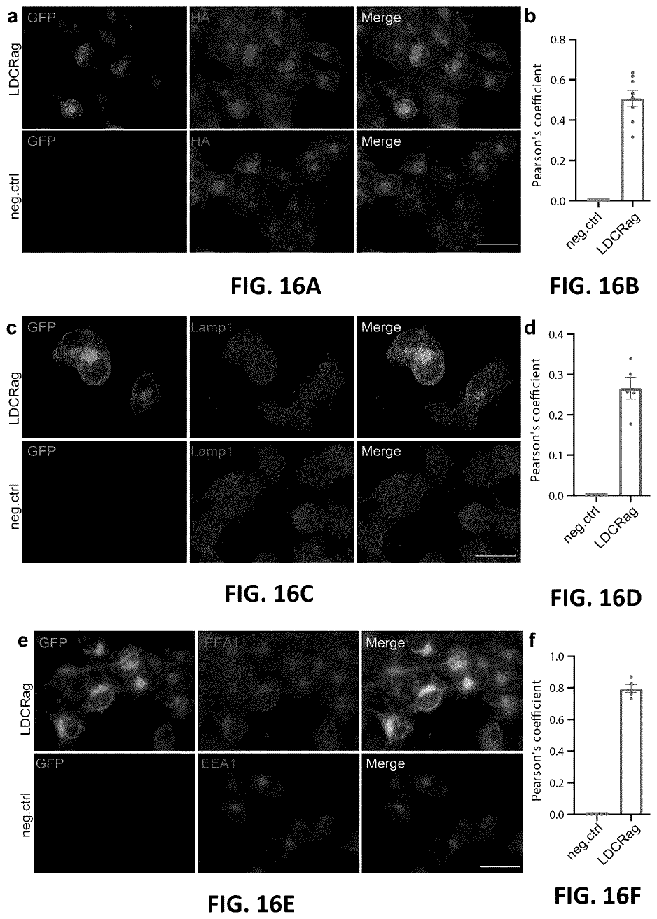

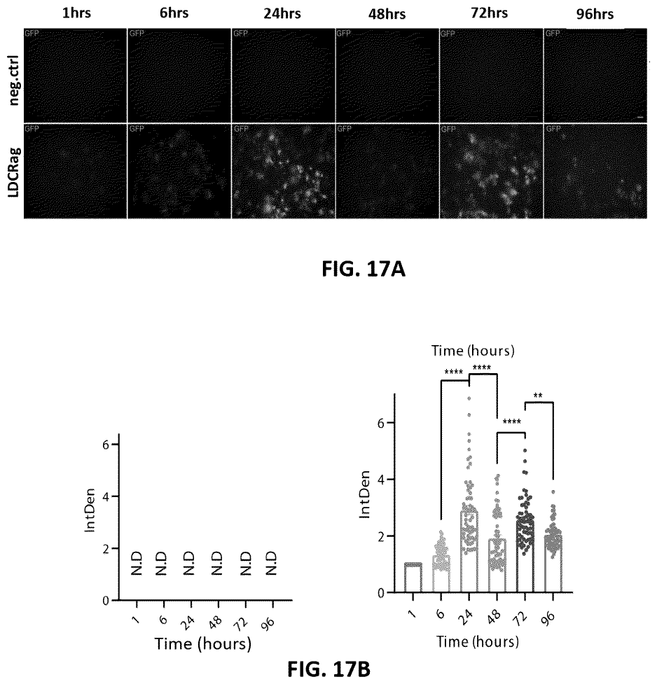

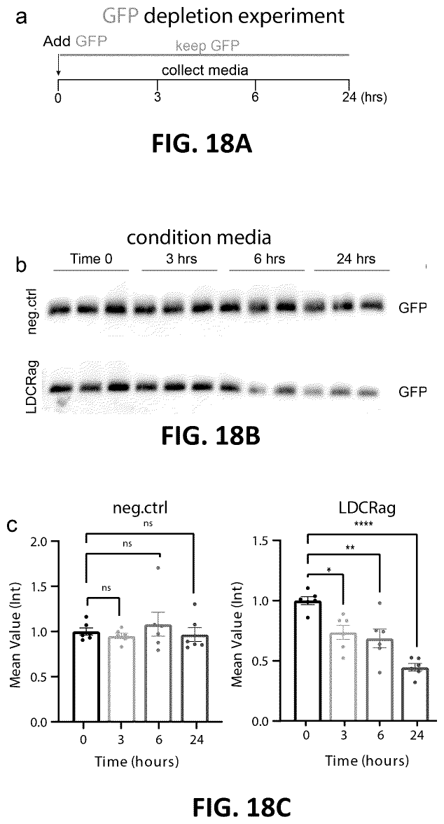

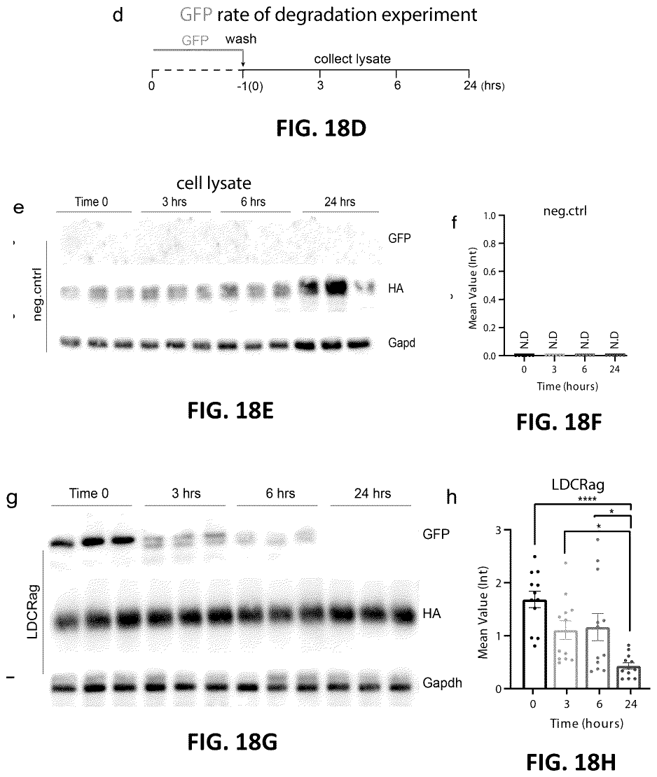

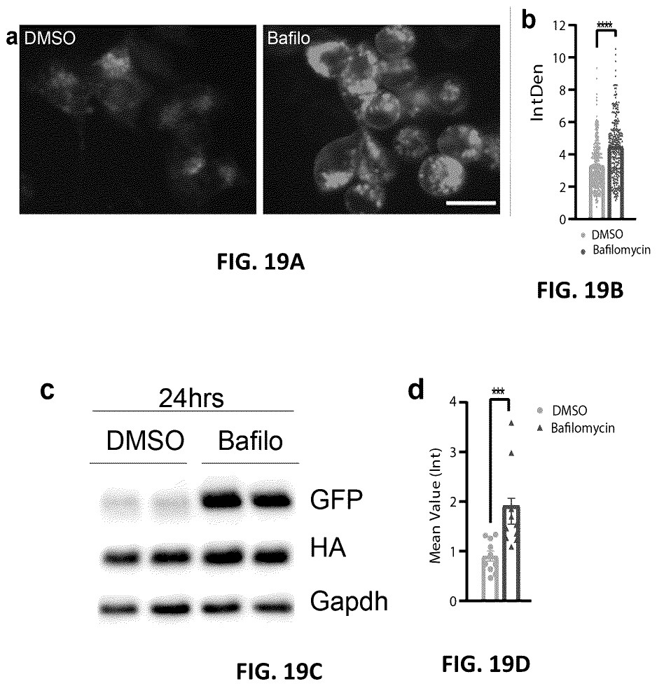

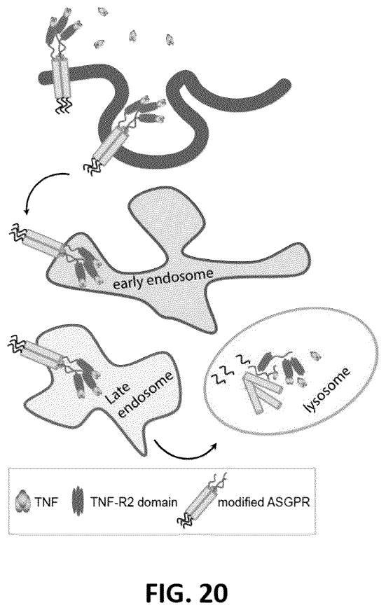

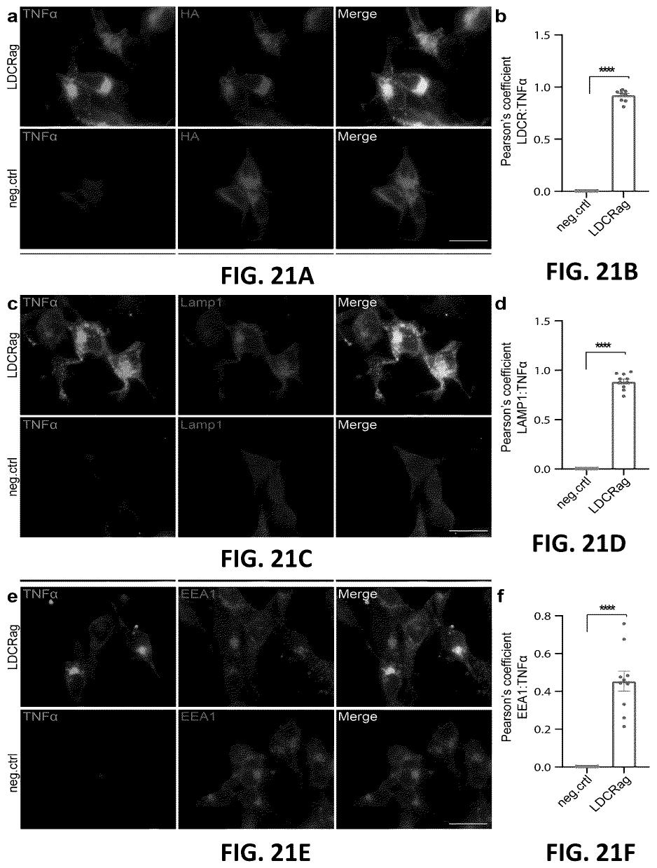

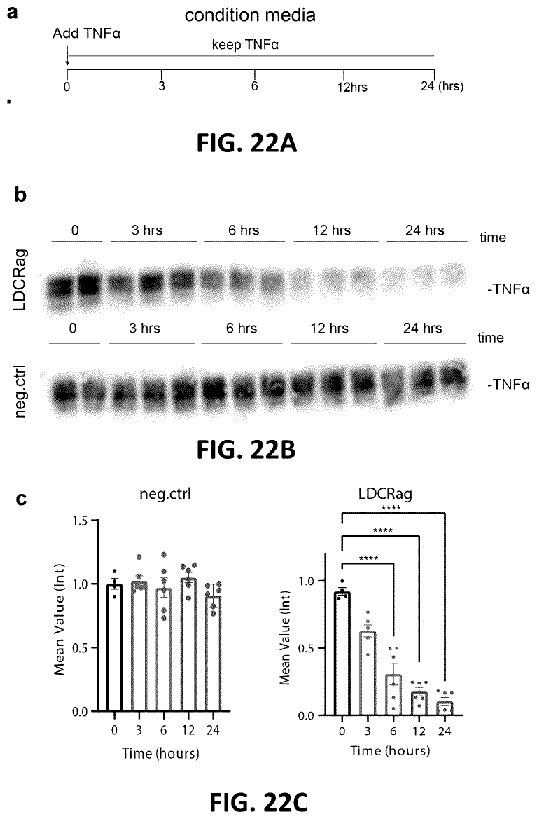

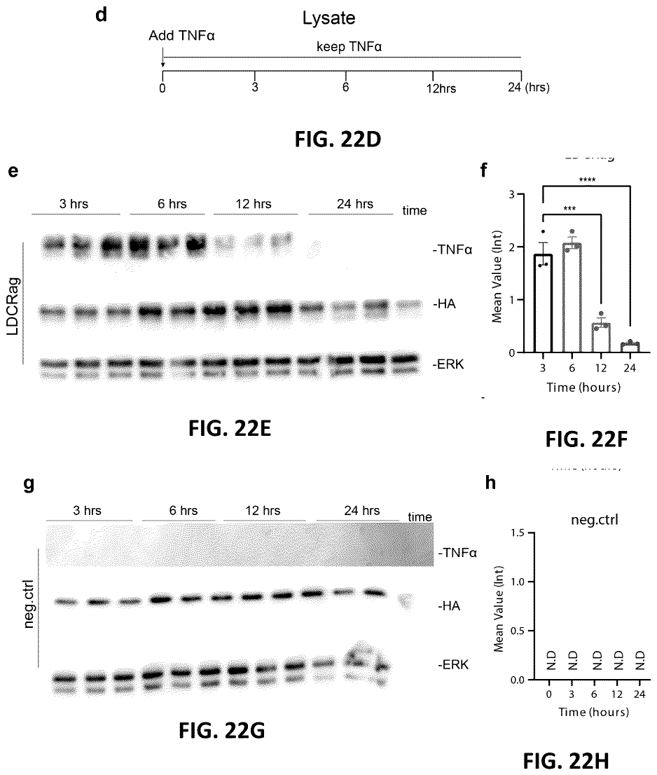

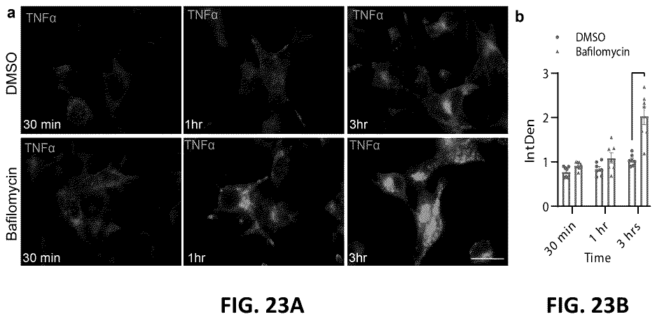

We have obtained four single-domain antibodies (VHH) targeting human E4 protein in collaboration with Hybrigenics Services. Hybrigenics Services platform utilized a synthetic hsd2Ab VVH Phage Display library of 3.109 clones that are expressed on the surface of M13 phage. Human lipidated E4 protein was incubated with the Phage Display for a total of three rounds. To control the enrichment of specific anti-human E4 VHHs during each round of Phage Display, an output/input ratio was measured and an ELISA for human E4 protein on the pool of each round was performed (FIG. 13A, FIG. 13B). Progressive enrichment during the cycle shows that the Phage Display conditions were appropriate, and 90 clones were analyzed from rounds 2 and 3 by non-absorbed phage ELISA, identifying 4 clones that bound to human ApoE protein (FIG. 13A, FIG. 13B). The sequences from the positive clones (A01-A04) were cloned into pHEN2 expression plasmid with an N-terminal Myc tag. Following the purification of recombinant A01-A04 the anti-human E4 VHH was analyzed on a non-adsorbed VHH ELISA which displayed binding to human lipidated ApoE4 protein (FIG. 12A, FIG. 12B, FIG. 12C, FIG. 14).

We cloned the anti-human E4 VHH sequence (A01-A04) to an expression vector containing a rabbit IgG Fc domain and purified them following the expression in HEK293t cells (SinoBiological). The purified anti-human E4 VHH fused to rabbit IgG Fc domain will be used for biochemical and histological analyses of binding to human E4. To determine the binding affinity (KD) for each anti-human E4 VHH, we will perform surface plasmon resonance with recombinant human lipidated E4 protein. To determine binding to human E4 protein, we will perform IF analysis of tau-tg mice expressing human E4 gene (tau-E4) brain sections previously analyzed with each anti-human E4 VHH and respective negative controls. Additional biochemical analysis will include immunoblotting and ELISA-based measurements of brain lysates from human AD and tau-E4 mice. To further validate the specificity of each anti-human E4 VHH, we will perform an IF analysis of tau-tg mice following the knockout of the APOE gene that was previously analyzed.

Following the characterization of the anti-human E4 VHH and identifying the most proficient binding to human E4 protein the VHH sequence will be subcloned into our LDCR. The four anti-human E4 LDCRs will be evaluated with lipidated human E4 protein labeled with pHrodoRed and analyzed with live imaging, flow cytometry, biochemical, and IF analyses detailed in Aim2a in HEK293t cells and primary astrocytes/neurons expressing human E4 gene.

Evaluate an anti-human ApoE LDCR to decrease ApoE in tau-E4 mice.

We aim to evaluate the LDCR's efficiency in reducing extracellular human E4 protein and decreasing AD-related pathologies in vivo. Previously, we demonstrated that tau-E4 mice exacerbated neuroinflammation, brain atrophy, and tau pathology2. Meanwhile, APOE gene knockout in tau-tg mice largely prevented brain atrophy and decreased tau-associated pathologies, providing a paradigm to evaluate decreasing E4 protein levels with our LDCRs2. To globally express the most proficient anti-human ApoE LDCR in the brain of tau-E4 mice, we will utilize AAVPHP.eB. We will further evaluate the efficacy of expressing anti-human ApoE LDCR in neurons or astrocytes by using synapsin or GFAP promoters respectively. To express the LDCRs and controls in the brains of tau-E4 mice, we will administer AAV PHP.eB serotype before and after disease onset and aged to 9.5 months and analyzed by histopathological, biochemical, and behavioral analyses. We will also determine the efficiency of the anti-human ApoE LDCR in decreasing E4 levels in tau-E4 mice by ELISA-based measurement in the CSF and brain lysates.

We may increase the surface levels of the LDCRs by slowing the rapid internalization. The signal for the rapid internalization of the MPRs has been localized to the cytoplasmic domains, and distinct mutations have been demonstrated to slow internalization significantly. Introducing these distinct mutations may increase cell surface binding to extracellular proteins, enhancing the degradation of extracellular proteins. We will identify the most proficient LDCR that decreases extracellular proteins following the flow cytometry analysis and measuring GFP protein levels in the conditioned medium. The anti-human E4 VHHs will specifically bind human ApoE and anti-E4 LDCR will decrease E4 levels in vitro and in vivo. We may find expressing the anti-E4 LDCR in astrocytes decreases intracellular E4 levels by binding newly synthesized E4 at the Golgi for lysosomal delivery. Based on this notion, we predict astrocytic anti-ApoE LDCR expression in tau-E4 mice will lead to a more significant decrease in E4 levels as astrocytes are the predominant cell type expressing E4. It is anticipated that decreasing E4 levels following anti-E4 LDCR expression will decrease tau-related pathologies and improve behavioral assessments.

The anti-human E4 VHHs may display inadequate binding to human E4 protein. In this scenario, we will generate scFv targeting human E4 in collaboration with Dr. Holtzman, who has generated a series of anti-human E4 antibodies. In addition, a potential complication is a high extracellular ApoE levels range ~9.09 ug/ml and we may find that expressing the LDCRs in neurons or astrocytes does not substantially decrease E4 protein levels. Here, we have generated additional VHHs targeting the proinflammatory molecules TNF-α that is elevated in human AD and emerging evidence suggests neutralizing TNF-α is a therapeutic strategy for AD. Moreover, TNF-α levels ranged from 50 to 100 μg/ml in CSF, making it more amenable to decreasing extracellular levels by expressing the anti-TNF-α LDCRs.

### Methods

To evaluate the role of lysosome in GFP degradation, HEK293t cells expressing different LDCRs treated with bafilomycin (500 nm) and 0.45 ug/ml GFP for 3 hrs and collected in the different time points for live imaging or immunoblot. To evaluate GFP localization in the lysosome, GFP labeled with fluorescent dye Phrodred. LDCRs expressing cells treated with GFP-Phrodo for 3 hrs and collected for high magnified imaging. For detection of the GFP in the condition media, HEK293t cells expressing different LDCRs and negative control treated with different concentration of the GFP for 3 hrs and condition media collected for immunoblot analysis

### Example 3—Targeted Extracellular Protein Degradation by Lysosome Using Modified Mannose 6 Phosphate Receptors

Targeted protein degradation (TPD) via lysosome provided new tools which can selectively target disease-causing protein for degradation. We designed new lysosomal degrading chimeric receptors (LDCRs) that target disease associated secretory and extracellular proteins for degradation. APOE4 (E4) gene is one of the genetic risk factors which is associated with the risk of Alzheimer's disease (AD). The exact mechanism which APOE4 increases the risk of AD remains to be fully understood. APOE4 can increase amyloid beta plaque deposition, increase tau protein phosphorylation, and increase inflammation. Doing immunotherapy against APOE4 in APP mice animal models decreased plaques in the cortex and hippocampus. Since doing other therapies such as immunotherapies have their own limitations, finding other approaches which be able to target and degrade brain APOE4 is still challenging. In this study, we used the mannose-6-phosphate receptors (M6PRs) to generate the LDCRs to target extracellular APOE4 for intracellular lysosomal degradation. There are two different types of M6PR receptors (FIG. 1, FIG. 2). At the trans-Golgi network, M6PRs bind to the mannose-6 phosphates (M6P) glycans on newly synthesized lysosomal enzymes for lysosomal delivery. The main majority of M6PRs (90%) recycle between the trans-Golgi, and early/late endosomes, but a fraction of the M6PRs (10%) traffic to the cell surface to bind and internalize extracellular lysosomal enzymes containing M6P glycans.

### Results

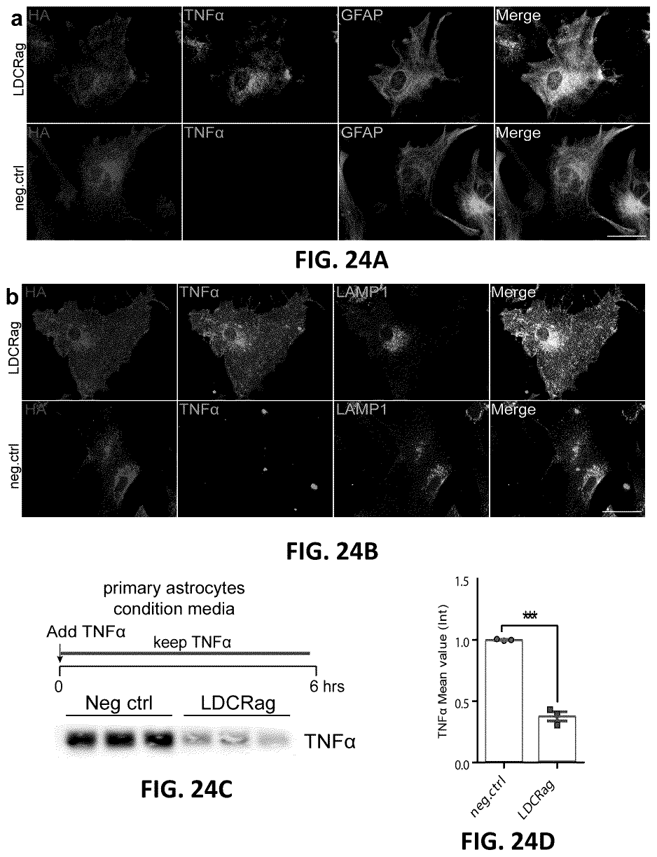

To build on our data showing that the LDCR effectively targets extracellular therapeutic targets for degradation in HEPA cells, we next assessed its effectiveness in astrocytes. Astrocytes are a type of glial cell in the brain with several critical roles, including regulating ionic homeostasis, metabolism, and brain inflammation. Primary astrocytes isolated from P4 mouse cortical brain regions were transduced with the LDCR targeting TNFα (LDCRag^{TNFα}) followed by treatment with biotinylated activated TNFα in the conditioned media. IF analysis with astrocytic glial fibrillary acidic protein (GFAP) marker revealed that primary astrocytes expressing LDCRag^{TNFα} bind TNFα in the conditioned media, as demonstrated by colocalization with anti-HA (FIG. 24A). Further, TNFα is colocalized with lysosomes (LAMP1) following the expression of the LDCRag^{TNFα} (FIG. 24B). Immunoblotting analysis of conditioned media from primary astrocytes expressing LDCRag^{TNFα} further revealed a significant decrease in TNFα levels relative to the negative control (FIG. 24C, FIG. 24D). These analyses demonstrate that the LDCR effectively traffics to the cell surface, binding to extracellular TNFα, which is targeted to the lysosome for degradation in diverse cell types.

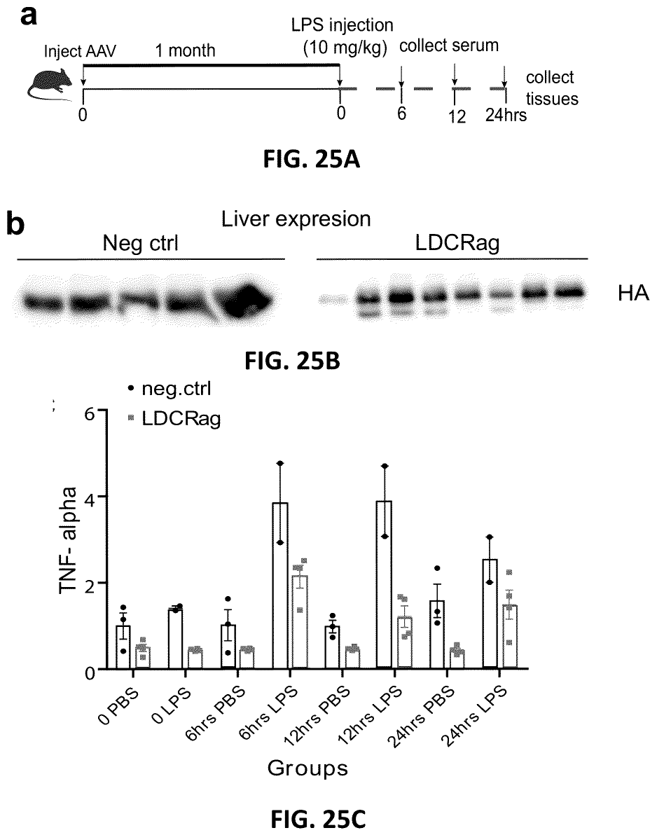

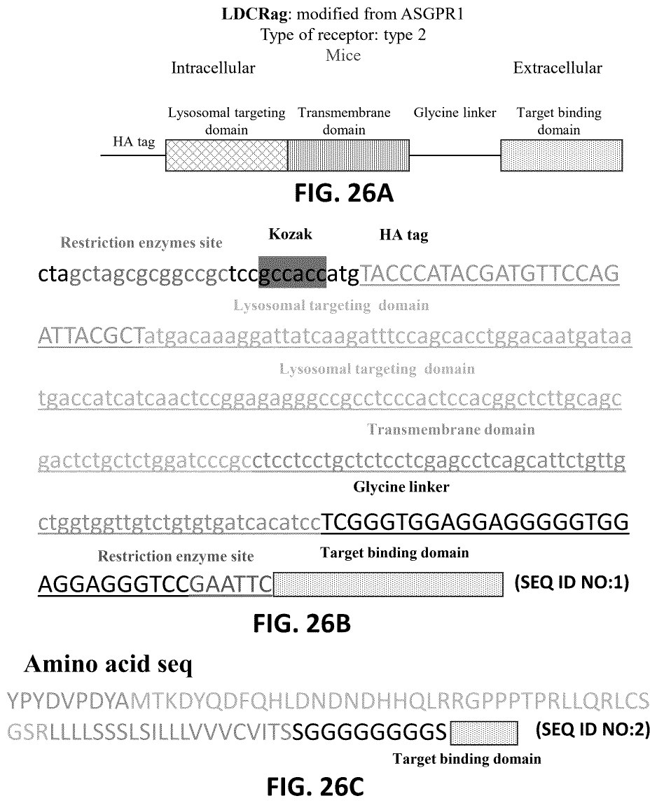

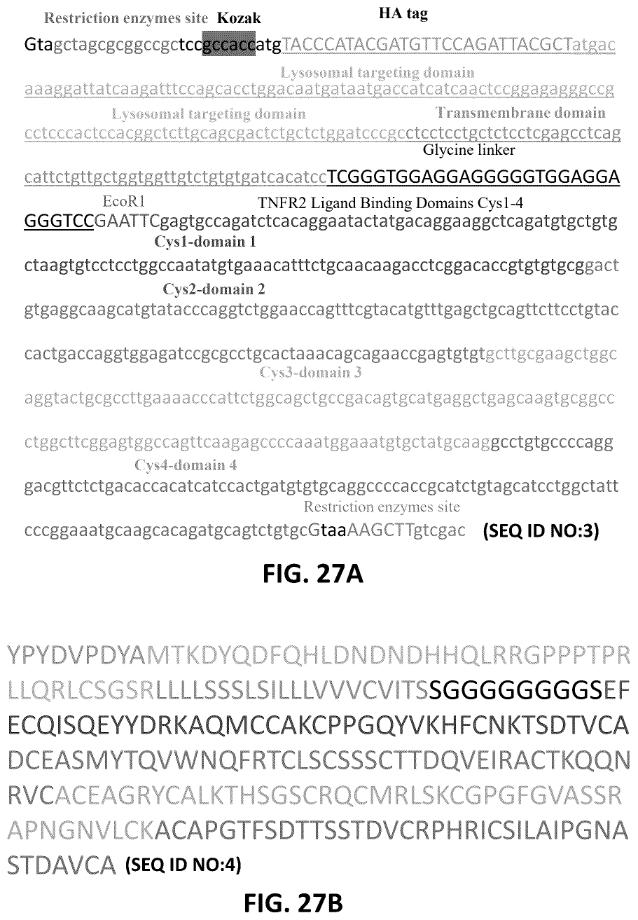

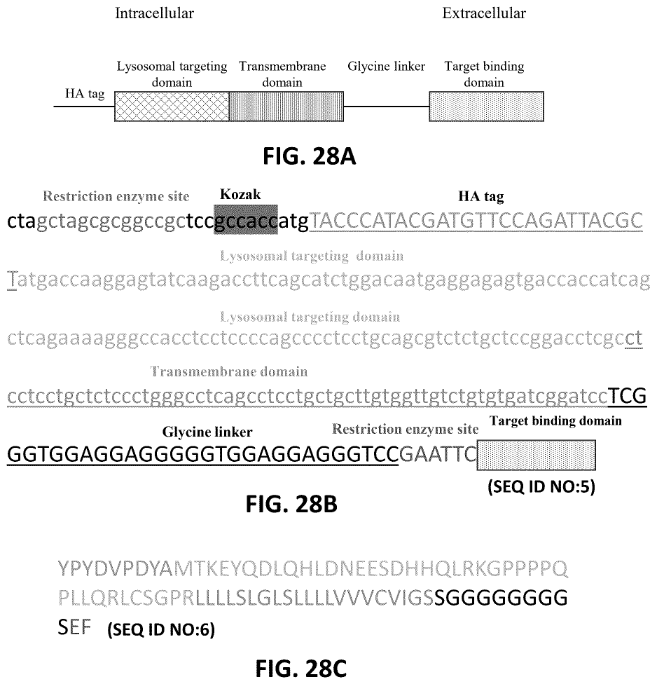

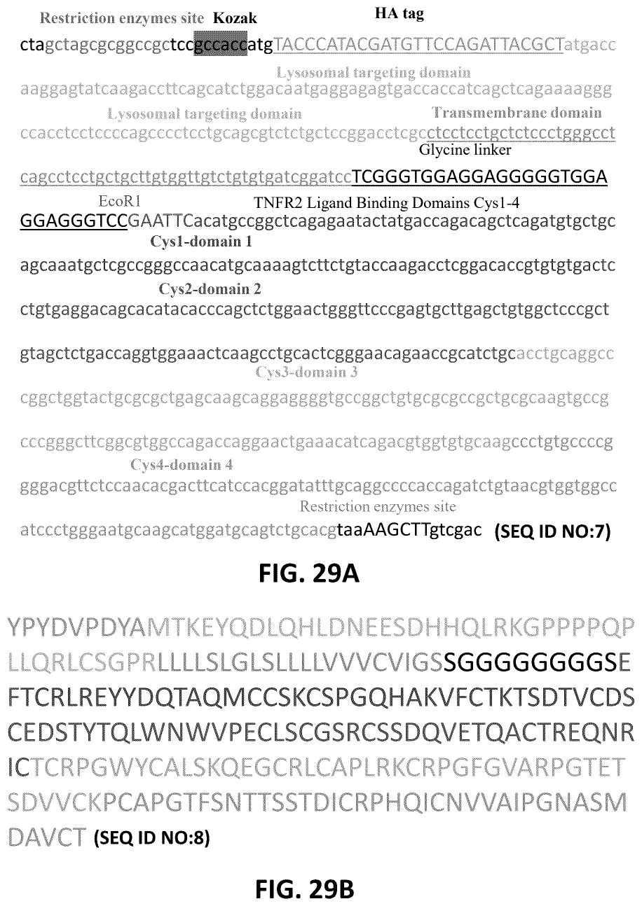

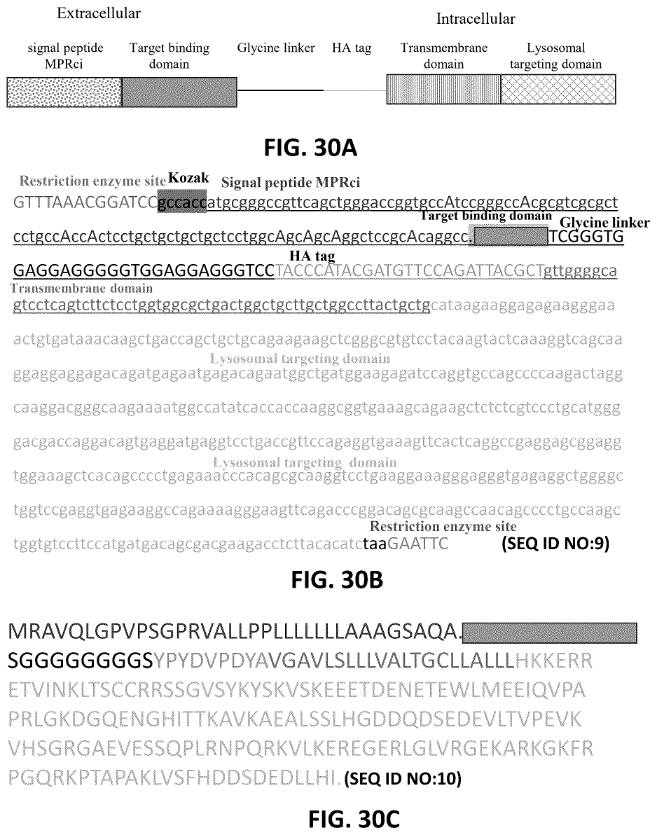

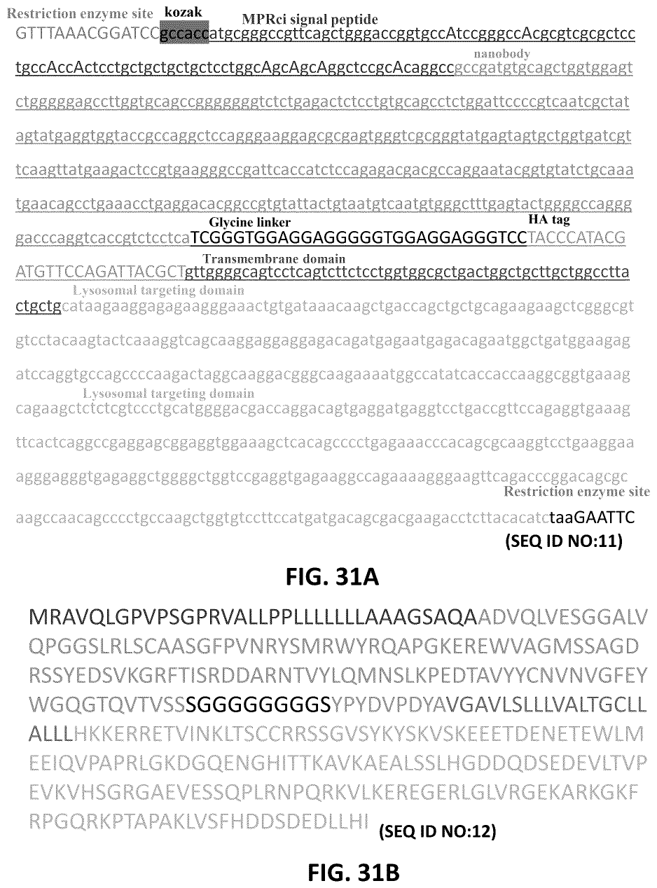

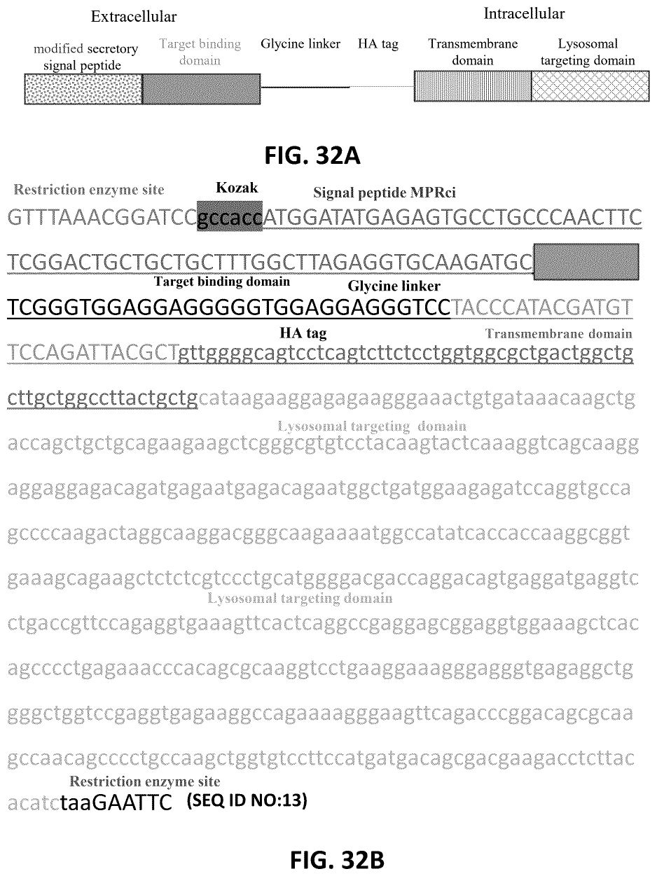

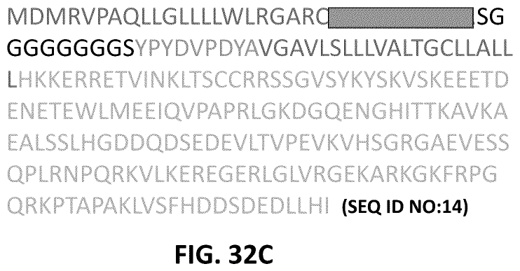

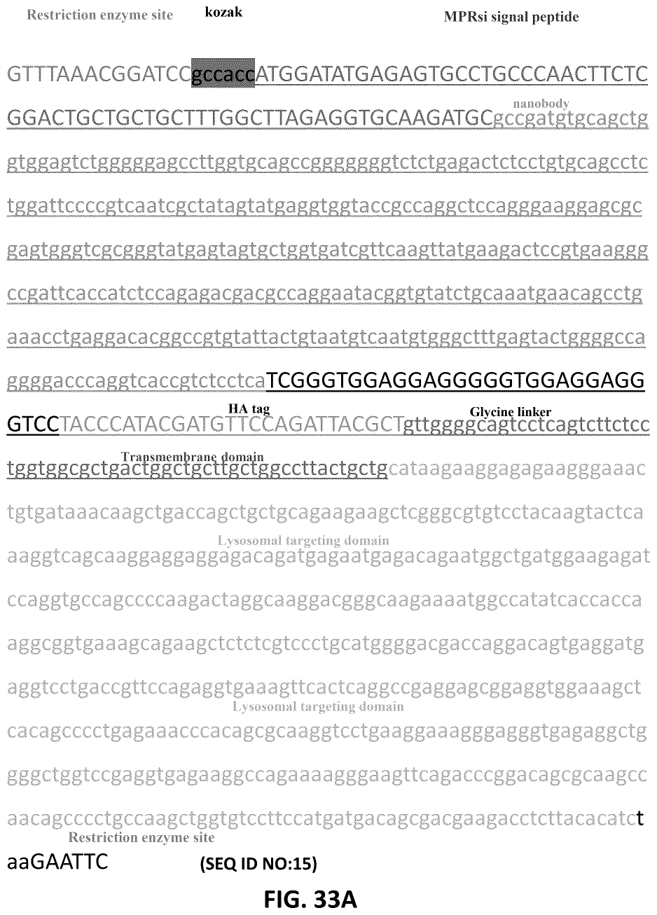

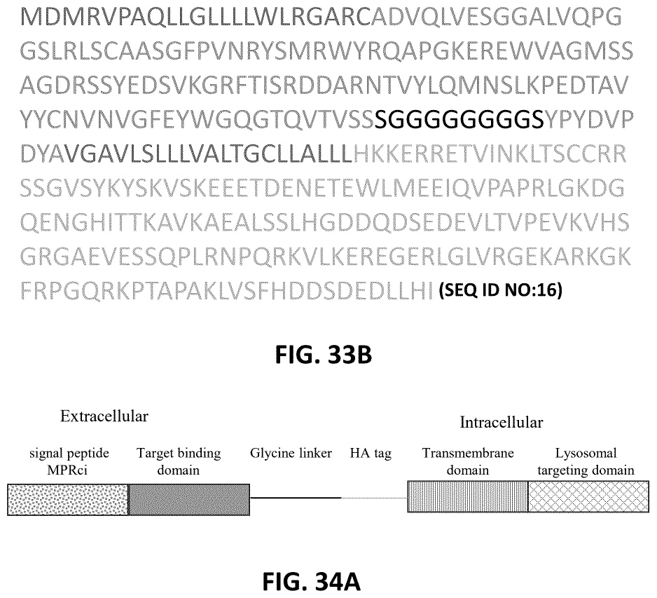

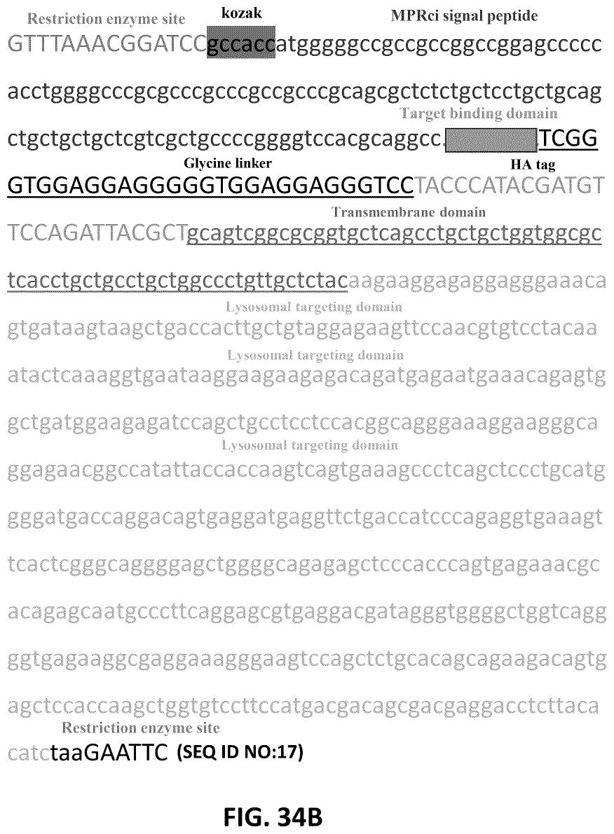

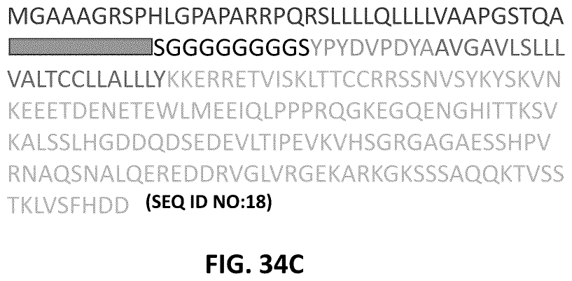

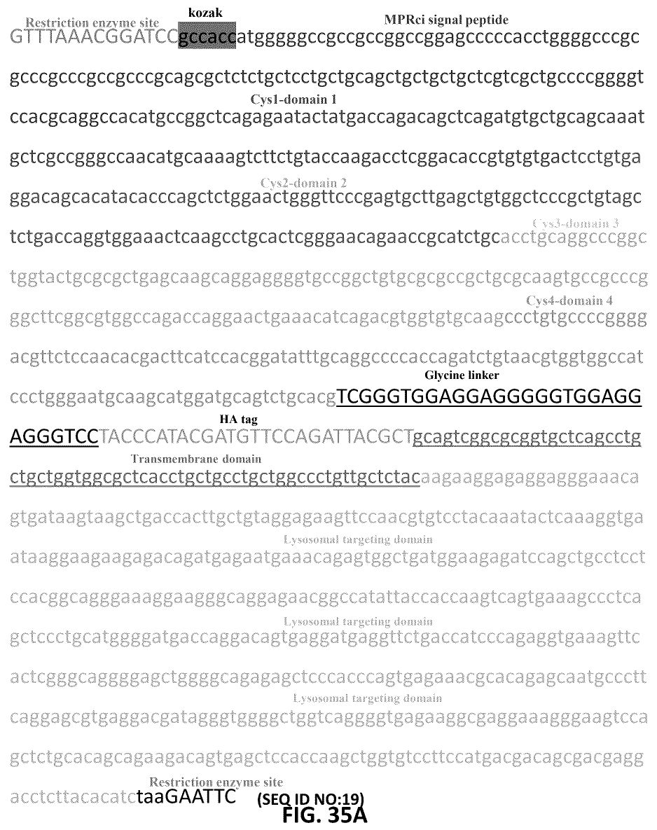

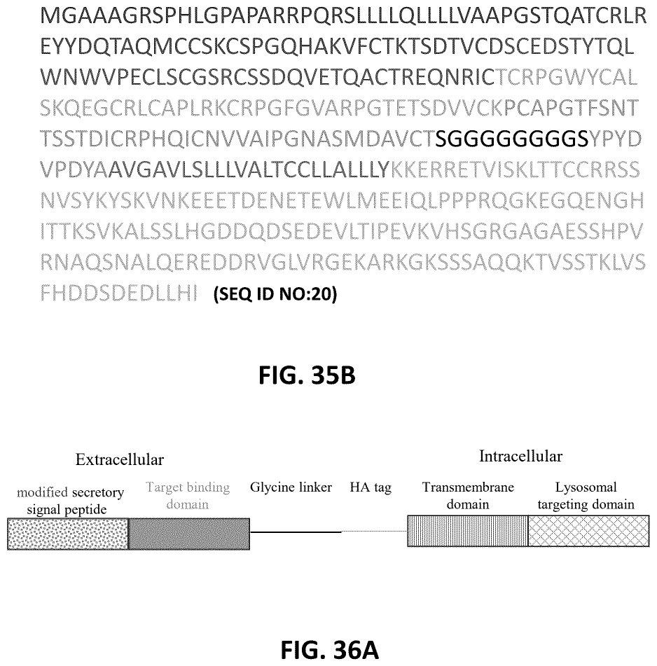

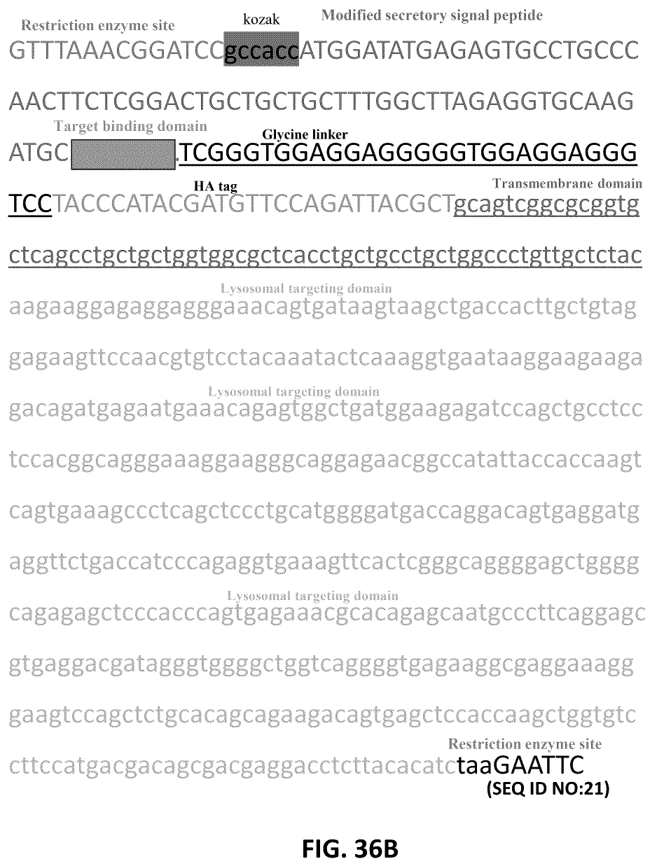

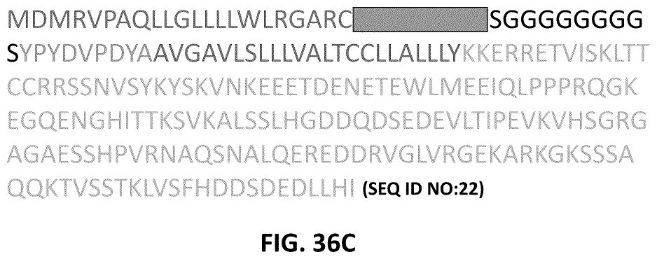

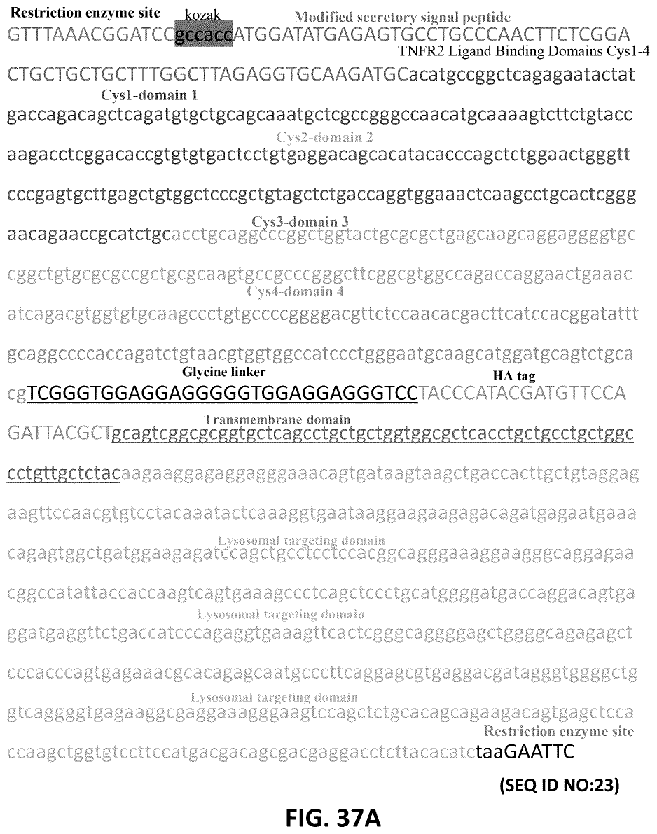

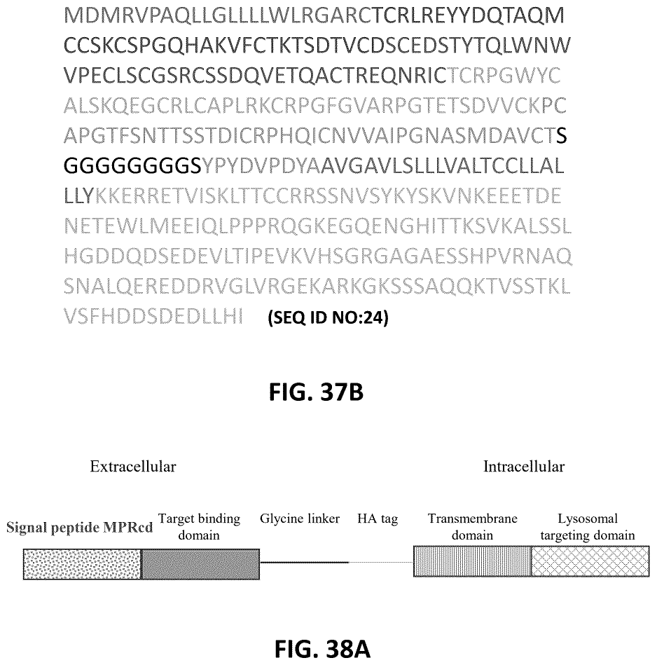

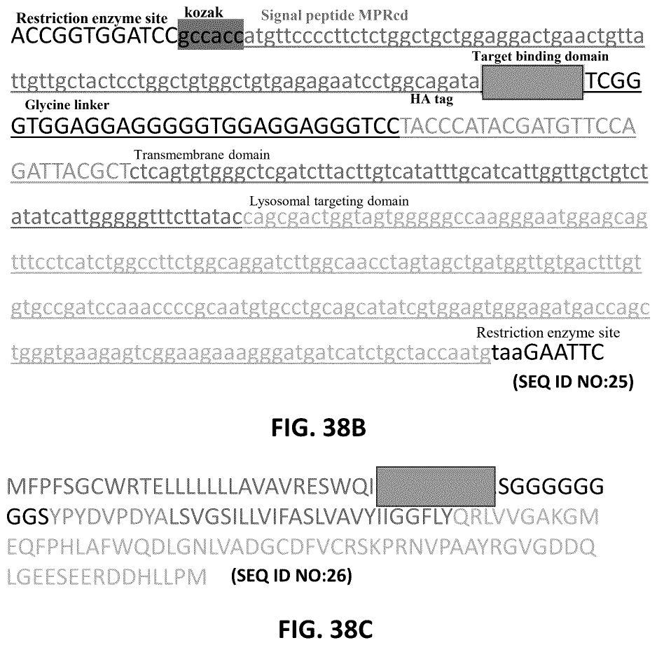

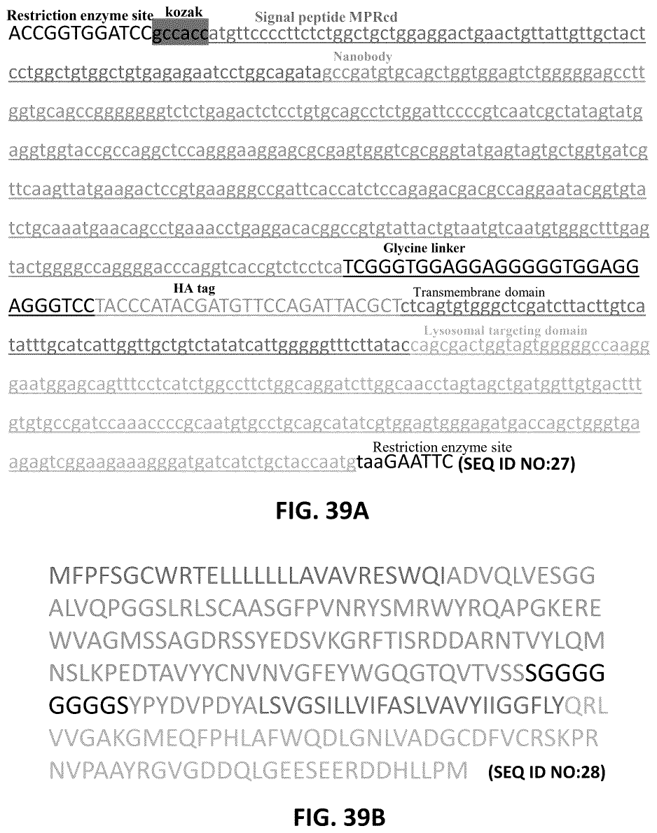

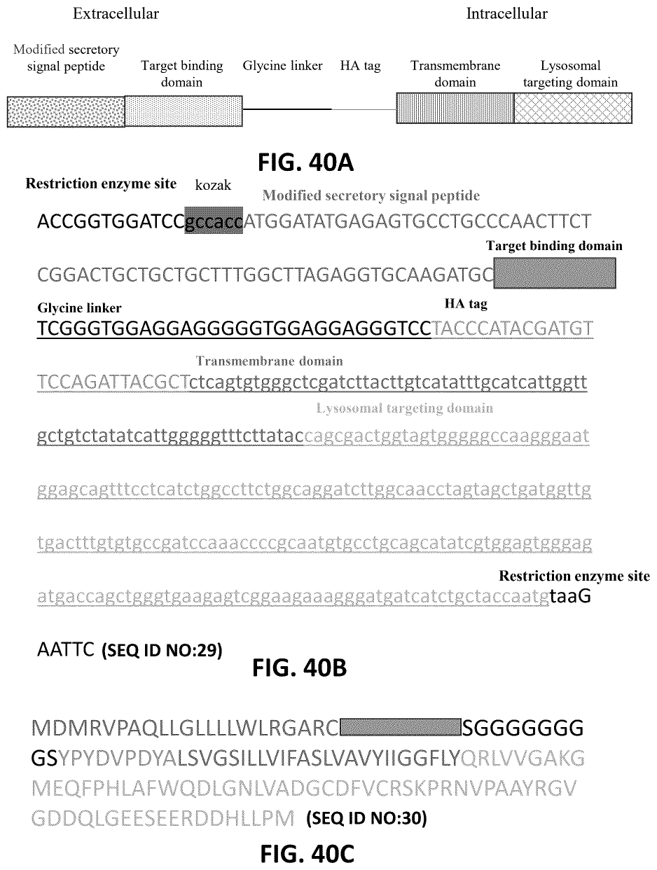

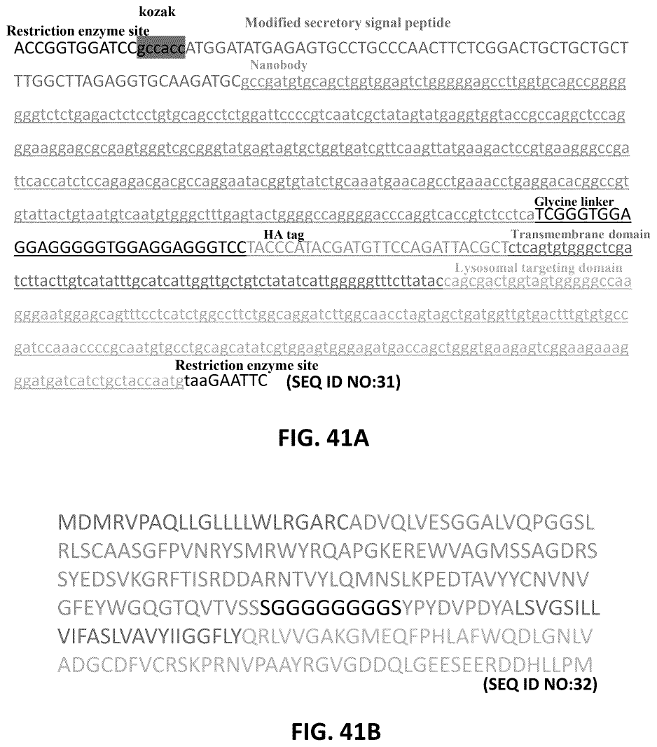

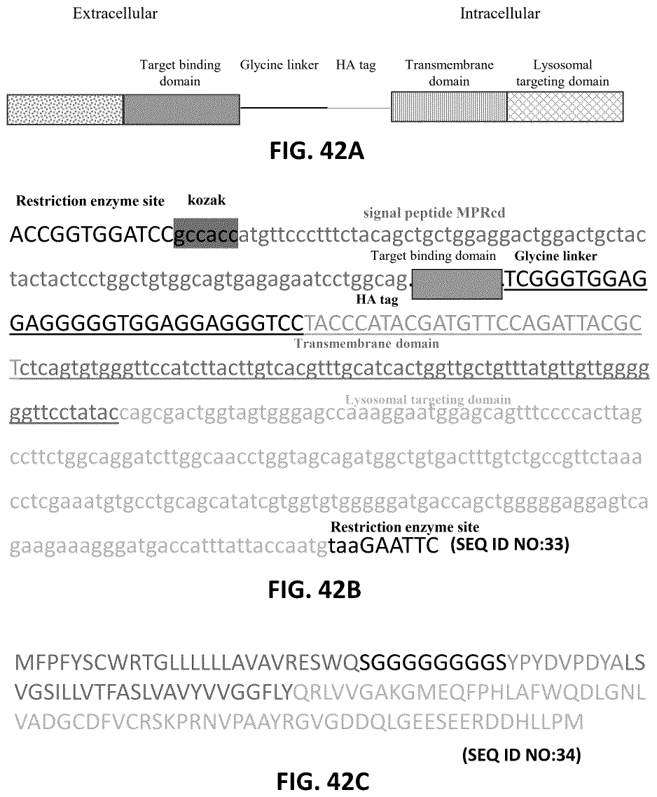

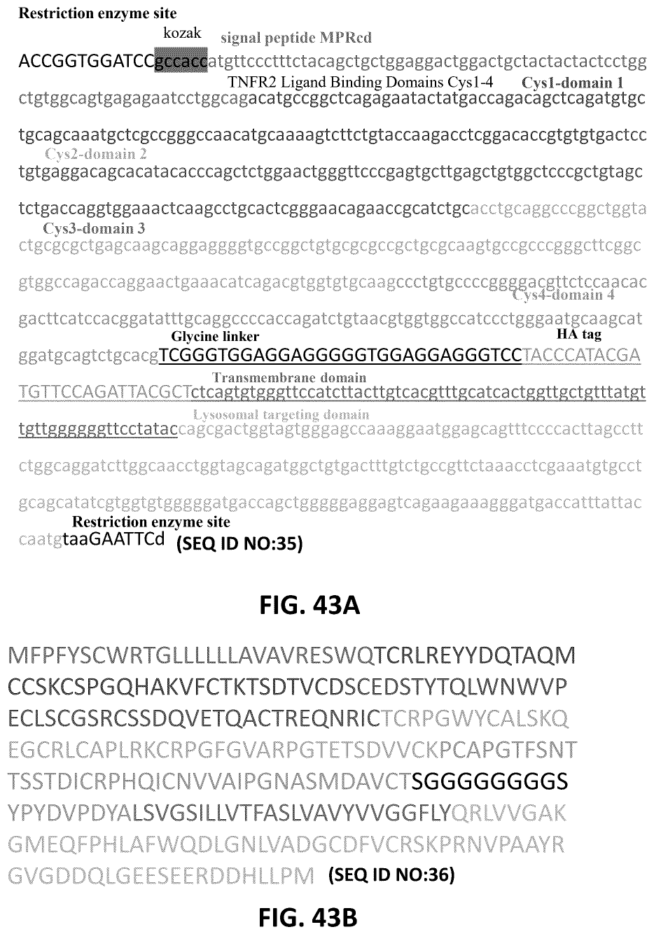

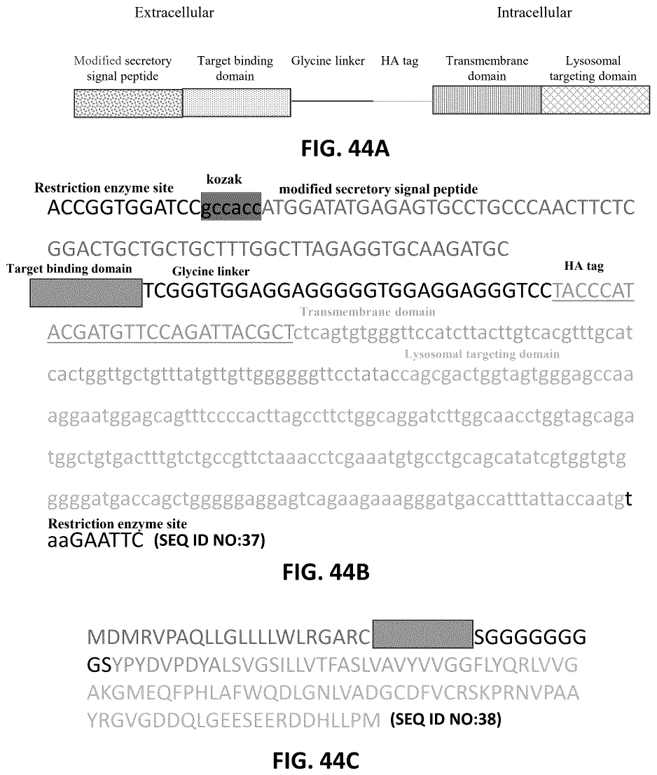

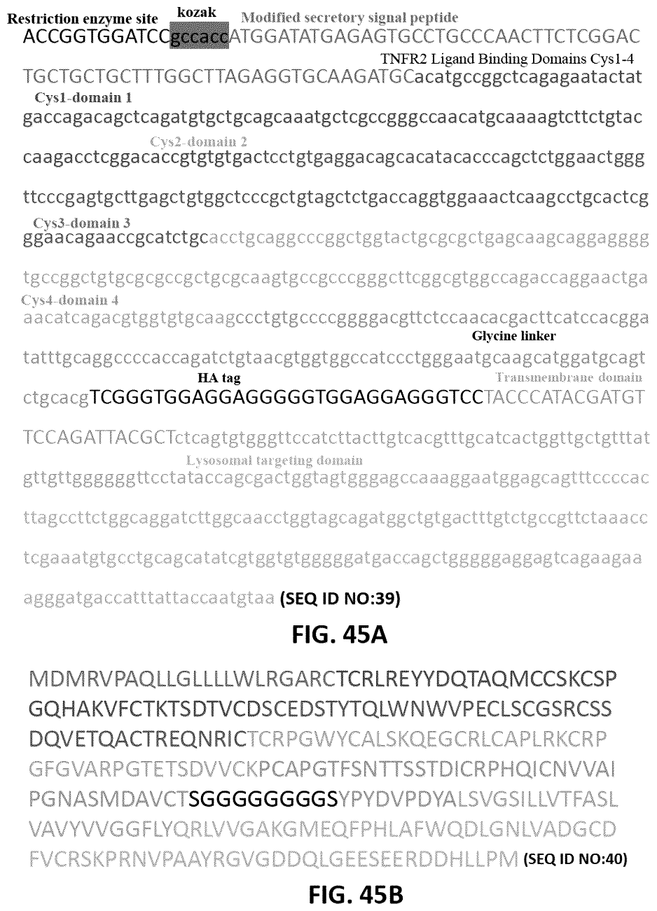

We next evaluated the effectiveness of LDCRag^{TNFα} in reducing circulating TNFα levels in an acute inflammatory model following the intraperitoneal (i.p.) administration of lipopolysaccharide (LPS, 10 mg) into 3- to 4-month-old C57BL/6 wild-type mice. To assess the ability of our LDCRag^{TNFα} to lower TNFα in vivo, we expressed LDCRag^{TNFα} or a negative control in hepatocytes using adeno-associated viral (AAV) gene therapy. Since hepatocytes break down large amounts of circulating proteins via lysosomal degradation, and AAV-based liver gene therapy has been clinically tested in patients with hemophilia B, we subcloned LDCRag^{TNFα} and the negative control into an AAV vector previously shown to target hepatocytes in vivo with a TBG promoter (Addgene). To determine the efficacy of LDCRag^{TNFα}, male and female C57BL/6 wild-type mice (n=4 per condition) were intravenously injected with AAV2/8 (1×10\hat{ }11 GC/mouse) encoding either LDCRag^{TNFα} or a negative control. The AAV2/8 serotype provides long-term expression in the mouse liver, beginning two weeks after injection and reaching optimal levels at four weeks. One month post-injection, the mice received intraperitoneal LPS, followed by blood collection at 0, 6, 12, and 24 hours, and were euthanized. Liver biopsies confirmed expression of LDCRagTNFα and the negative control by immunoblotting (FIG. 25A). Furthermore, ELISA analysis showed a significant increase in circulating TNFα in serum following LPS administration at 6 and 12 hours in negative control mice, which was markedly reduced by the expression of LDCRag^{TNFα} (FIG. 25B).

### Example 3—Chimeric Receptor Constructs

TABLE 1
DNA Sequence Chimeric Receptor Constructs

| | |
|---|---|
| SEQ ID NO | Sequence |

chimeric receptor constructs from modified ASGPR1 receptor (LDCRag)

| | |
|---|---|
| SEQ ID | ctagctagcgcggccgctccgccaccatgTACCCATACGATGTTCCAGATTACGCT |
| NO: 1 | atgacaaaggattatcaagatttccagcacctggacaatgataatgaccatcatcaactccggagag |
| | ggccgcctcccactccacggctcttgcagcgactctgctctggatcccgcctcctcctgctctcctcgag |
| | cctcagcattctgttgctggtggttgtctgtgtgatcacatccTCGGGTGGAGGAGGGGGT |
| | GGAGGAGGGTCCGAATTC |
| SEQ ID | GtagctagcgcggccgctccgccaccatgTACCCATACGATGTTCCAGATTACGC |
| NO: 3 | Tatgacaaaggattatcaagatttccagcacctggacaatgataatgaccatcatcaactccggaga |
| | gggccgcctcccactccacggctcttgcagcgactctgctctggatcccgcctcctcctgctctcctcga |
| | gcctcagcattctgttgctggtggttgtctgtgtgatcacatccTCGGGTGGAGGAGGGGGT |
| | GGAGGAGGGTCCGAATTCgagtgccagatctcacaggaatactatgacaggaaggctc |
| | agatgtgctgtgctaagtgtcctcctggccaatatgtgaaacatttctgcaacaagacctcggacaccg |
| | tgtgtgcggactgtgaggcaagcatgtatacccaggtctggaaccagtttcgtacatgtttgagctgca |
| | gttcttcctgtaccactgaccaggtggagatccgcgcctgcactaaacagcagaaccgagtgtgtgct |
| | tgcgaagctggcaggtactgcgccttgaaaacccattctggcagctgccgacagtgcatgaggctga |
| | gcaagtgcggccctggcttcggagtggccagttcaagagccccaaatggaaatgtgctatgcaagg |
| | cctgtgccccagggacgttctctgacaccacatcatccactgatgtgtgcaggccccaccgcatctgta |
| | gcatcctggctattcccggaaatgcaagcacagatgcagtctgtgcGtaaAAGCTTgtcgac |
| SEQ ID | ctagctagcgcggccgctccgccaccatgTACCCATACGATGTTCCAGATTACGCT |
| NO: 5 | atgaccaaggagtatcaagaccttcagcatctggacaatgaggagagtgaccaccatcagctcaga |
| | aaagggccacctcctccccagcccctcctgcagcgtctctgctccggacctcgcctcctcctgctctcc |
| | ctgggcctcagcctcctgctgcttgtggttgtctgtgtgatcggatccTCGGGTGGAGGAGGG |
| | GGTGGAGGAGGGTCCGAATTC |
| SEQ ID | ctagctagcgcggccgctccgccaccatgTACCCATACGATGTTCCAGATTACGCT |
| NO: 7 | atgaccaaggagtatcaagaccttcagcatctggacaatgaggagagtgaccaccatcagctcaga |
| | aaagggccacctcctccccagcccctcctgcagcgtctctgctccggacctcgcctcctcctgctctcc |
| | ctgggcctcagcctcctgctgcttgtggttgtctgtgtgatcggatccTCGGGTGGAGGAGGG |
| | GGTGGAGGAGGGTCCGAATTCacatgccggctcagagaatactatgaccagacagc |
| | tcagatgtgctgcagcaaatgctcgccgggccaacatgcaaaagtcttctgtaccaagacctcggac |
| | accgtgtgtgactcctgtgaggacagcacatacacccagctctggaactgggttcccgagtgcttgag |
| | ctgtggctcccgctgtagctctgaccaggtggaaactcaagcctgcactcgggaacagaaccgcatc |
| | tgcacctgcaggcccggctggtactgcgcgctgagcaagcaggaggggtgccggctgtgcgcgcc |
| | gctgcgcaagtgccgcccgggcttcggcgtggccagaccaggaactgaaacatcagacgtggtgt |
| | gcaagccctgtgccccggggacgttctccaacacgacttcatccacggatatttgcaggccccacca |
| | gatctgtaacgtggtggccatccctgggaatgcaagcatggatgcagtctgcacgtaaAAGCTTg |
| | tcgac |

Mice chimeric receptor constructs from modified MPRs receptors
(LDCRci, LDCRsi)

| | |
|---|---|
| SEQ ID | GTTTAAACGGATCCgccaccatgcgggccgttcagctgggaccggtgccAtccgggccAc |
| NO: 9 | gcgtcgcgctcctgccAccActcctgctgctgctgctcctggcAgcAgcAggctccgcAcaggcc.. |
| | ...............TCGGGTGGAGGAGGGGGTGGAGGAGGGTCCTACCCATAC |
| | GATGTTCCAGATTACGCTgttggggcagtcctcagtcttctcctggtggcgctgactggctg |
| | cttgctggccttactgctgcataagaaggagagaagggaaactgtgataaacaagctgaccagctg |
| | ctgcagaagaagctcgggcgtgtcctacaagtactcaaaggtcagcaaggaggaggagacagat |
| | gagaatgagacagaatggctgatggaagagatccaggtgccagccccaagactaggcaaggac |
| | gggcaagaaaatggccatatcaccaccaaggcggtgaaagcagaagctctctcgtccctgcatgg |
| | ggacgaccaggacagtgaggatgaggtcctgaccgttccagaggtgaaagttcactcaggccgag |
| | gagcggaggtggaaagctcacagcccctgagaaacccacagcgcaaggtcctgaaggaaaggg |
| | agggtgagaggctggggctggtccgaggtgagaaggccagaaaagggaagttcagacccggac |
| | agcgcaagccaacagcccctgccaagctggtgtccttccatgatgacagcgacgaagacctcttac |
| | acatctaaGAATTC |
| SEQ ID | GTTTAAACGGATCCgccaccatgcgggccgttcagctgggaccggtgccAtccgggccAc |
| NO: 11 | gcgtcgcgctcctgccAccActcctgctgctgctgctcctggcAgcAgcAggctccgcAcaggccg |
| | ccgatgtgcagctggtggagtctgggggagccttggtgcagccgggggggtctctgagactctcctgt |
| | gcagcctctggattccccgtcaatcgctatagtatgaggtggtaccgccaggctccagggaaggagc |
| | gcgagtgggtcgcgggtatgagtagtgctggtgatcgttcaagttatgaagactccgtgaagggccga |
| | ttcaccatctccagagacgacgccaggaatacggtgtatctgcaaatgaacagcctgaaacctgag |
| | gacacggccgtgtattactgtaatgtcaatgtgggctttgagtactggggccaggggacccaggtcac |
| | cgtctcctcaTCGGGTGGAGGAGGGGGTGGAGGAGGGTCCTACCCATAC |
| | GATGTTCCAGATTACGCTgttggggcagtcctcagtcttctcctggtggcgctgactggctg |
| | cttgctggccttactgctgcataagaaggagagaagggaaactgtgataaacaagctgaccagctg |
| | ctgcagaagaagctcgggcgtgtcctacaagtactcaaaggtcagcaaggaggaggagacagat |
| | gagaatgagacagaatggctgatggaagagatccaggtgccagccccaagactaggcaaggac |
| | gggcaagaaaatggccatatcaccaccaaggcggtgaaagcagaagctctctcgtccctgcatgg |
| | ggacgaccaggacagtgaggatgaggtcctgaccgttccagaggtgaaagttcactcaggccgag |
| | gagcggaggtggaaagctcacagcccctgagaaacccacagcgcaaggtcctgaaggaaaggg |
| | agggtgagaggctggggctggtccgaggtgagaaggccagaaaagggaagttcagacccggac |
| | agcgcaagccaacagcccctgccaagctggtgtccttccatgatgacagcgacgaagacctcttac |
| | acatctaaGAATTC |
| SEQ ID | GTTTAAACGGATCCgccaccATGGATATGAGAGTGCCTGCCCAACTTC |
| NO: 13 | TCGGACTGCTGCTGCTTTGGCTTAGAGGTGCAAGATGC.................TC |
| | GGGTGGAGGAGGGGGTGGAGGAGGGTCCTACCCATACGATGTTCC |
| | AGATTACGCTgttggggcagtcctcagtcttctcctggtggcgctgactggctgcttgctggcctta |
| | ctgctgcataagaaggagagaagggaaactgtgataaacaagctgaccagctgctgcagaagaa |
| | gctcgggcgtgtcctacaagtactcaaaggtcagcaaggaggaggagacagatgagaatgagac |
| | agaatggctgatggaagagatccaggtgccagccccaagactaggcaaggacgggcaagaaaa |
| | tggccatatcaccaccaaggcggtgaaagcagaagctctctcgtccctgcatggggacgaccagg |
| | acagtgaggatgaggtcctgaccgttccagaggtgaaagttcactcaggccgaggagcggaggtg |
| | gaaagctcacagcccctgagaaacccacagcgcaaggtcctgaaggaaagggagggtgagag |
| | gctggggctggtccgaggtgagaaggccagaaaagggaagttcagacccggacagcgcaagcc |
| | aacagcccctgccaagctggtgtccttccatgatgacagcgacgaagacctcttacacatctaaGA |
| | ATTC |
| SEQ ID | GTTTAAACGGATCCgccaccATGGATATGAGAGTGCCTGCCCAACTTC |
| NO: 15 | TCGGACTGCTGCTGCTTTGGCTTAGAGGTGCAAGATGCgccgatgtgcag |
| | ctggtggagtctgggggagccttggtgcagccgggggggtctctgagactctcctgtgcagcctctgg |
| | attccccgtcaatcgctatagtatgaggtggtaccgccaggctccagggaaggagcgcgagtgggtc |
| | gcgggtatgagtagtgctggtgatcgttcaagttatgaagactccgtgaagggccgattcaccatctcc |
| | agagacgacgccaggaatacggtgtatctgcaaatgaacagcctgaaacctgaggacacggccgt |
| | gtattactgtaatgtcaatgtgggctttgagtactggggccaggggacccaggtcaccgtctcctcaTC |
| | GGGTGGAGGAGGGGGTGGAGGAGGGTCCTACCCATACGATGTTCC |
| | AGATTACGCTgttggggcagtcctcagtcttctcctggtggcgctgactggctgcttgctggcctta |
| | ctgctgcataagaaggagagaagggaaactgtgataaacaagctgaccagctgctgcagaagaa |
| | gctcgggcgtgtcctacaagtactcaaaggtcagcaaggaggaggagacagatgagaatgagac |
| | agaatggctgatggaagagatccaggtgccagccccaagactaggcaaggacgggcaagaaaa |
| | tggccatatcaccaccaaggcggtgaaagcagaagctctctcgtccctgcatggggacgaccagg |
| | acagtgaggatgaggtcctgaccgttccagaggtgaaagttcactcaggccgaggagcggaggtg |
| | gaaagctcacagcccctgagaaacccacagcgcaaggtcctgaaggaaagggagggtgagag |
| | gctggggctggtccgaggtgagaaggccagaaaagggaagttcagacccggacagcgcaagcc |
| | aacagcccctgccaagctggtgtccttccatgatgacagcgacgaagacctcttacacatctaaGA |
| | ATTC |

Human chimeric receptor constructs from modified MPRs receptors
(LDCRci, LDCRsi)

| | |
|---|---|
| SEQ ID | GTTTAAACGGATCCgccaccatgggggccgccgccggccggagcccccacctggggcc |
| NO: 17 | cgcgcccgcccgccgcccgcagcgctctctgctcctgctgcagctgctgctgctcgtcgctgccccgg |
| | ggtccacgcaggcc................TCGGGTGGAGGAGGGGGTGGAGGAGGG |
| | TCCTACCCATACGATGTTCCAGATTACGCTgcagtcggcgcggtgctcagcctg |
| | ctgctggtggcgctcacctgctgcctgctggccctgttgctctacaagaaggagaggagggaaacag |
| | tgataagtaagctgaccacttgctgtaggagaagttccaacgtgtcctacaaatactcaaaggtgaat |
| | aaggaagaagagacagatgagaatgaaacagagtggctgatggaagagatccagctgcctcctc |
| | cacggcagggaaaggaagggcaggagaacggccatattaccaccaagtcagtgaaagccctca |
| | gctccctgcatggggatgaccaggacagtgaggatgaggttctgaccatcccagaggtgaaagttc |
| | actcgggcaggggagctggggcagagagctcccacccagtgagaaacgcacagagcaatgccc |
| | ttcaggagcgtgaggacgatagggtggggctggtcaggggtgagaaggcgaggaaagggaagtc |
| | cagctctgcacagcagaagacagtgagctccaccaagctggtgtccttccatgacgacagcgacga |
| | ggacctcttacacatctaaGAATTC |
| SEQ ID | GTTTAAACGGATCCgccaccatgggggccgccgccggccggagcccccacctggggcc |
| NO: 19 | cgcgcccgcccgccgcccgcagcgctctctgctcctgctgcagctgctgctgctcgtcgctgccccgg |
| | ggtccacgcaggccacatgccggctcagagaatactatgaccagacagctcagatgtgctgcagc |
| | aaatgctcgccgggccaacatgcaaaagtcttctgtaccaagacctcggacaccgtgtgtgactcctg |
| | tgaggacagcacatacacccagctctggaactgggttcccgagtgcttgagctgtggctcccgctgta |
| | gctctgaccaggtggaaactcaagcctgcactcgggaacagaaccgcatctgcacctgcaggccc |
| | ggctggtactgcgcgctgagcaagcaggaggggtgccggctgtgcgcgccgctgcgcaagtgccg |
| | cccgggcttcggcgtggccagaccaggaactgaaacatcagacgtggtgtgcaagccctgtgcccc |
| | ggggacgttctccaacacgacttcatccacggatatttgcaggccccaccagatctgtaacgtggtgg |
| | ccatccctgggaatgcaagcatggatgcagtctgcacgTCGGGTGGAGGAGGGGGTG |
| | GAGGAGGGTCCTACCCATACGATGTTCCAGATTACGCTgcagtcggcgcg |
| | gtgctcagcctgctgctggtggcgctcacctgctgcctgctggccctgttgctctacaagaaggagagg |
| | agggaaacagtgataagtaagctgaccacttgctgtaggagaagttccaacgtgtcctacaaatact |
| | caaaggtgaataaggaagaagagacagatgagaatgaaacagagtggctgatggaagagatcc |
| | agctgcctcctccacggcagggaaaggaagggcaggagaacggccatattaccaccaagtcagt |
| | gaaagccctcagctccctgcatggggatgaccaggacagtgaggatgaggttctgaccatcccaga |
| | ggtgaaagttcactcgggcaggggagctggggcagagagctcccacccagtgagaaacgcacag |
| | agcaatgcccttcaggagcgtgaggacgatagggtggggctggtcaggggtgagaaggcgagga |
| | aagggaagtccagctctgcacagcagaagacagtgagctccaccaagctggtgtccttccatgacg |
| | acagcgacgaggacctcttacacatctaaGAATTC |
| SEQ ID | GTTTAAACGGATCCgccaccATGGATATGAGAGTGCCTGCCCAACTTC |
| NO: 21 | TCGGACTGCTGCTGCTTTGGCTTAGAGGTGCAAGATGC................. |
| | TCGGGTGGAGGAGGGGGTGGAGGAGGGTCCTACCCATACGATGTT |
| | CCAGATTACGCTgcagtcggcgcggtgctcagcctgctgctggtggcgctcacctgctgcctg |
| | ctggccctgttgctctacaagaaggagaggagggaaacagtgataagtaagctgaccacttgctgta |
| | ggagaagttccaacgtgtcctacaaatactcaaaggtgaataaggaagaagagacagatgagaat |
| | gaaacagagtggctgatggaagagatccagctgcctcctccacggcagggaaaggaagggcag |
| | gagaacggccatattaccaccaagtcagtgaaagccctcagctccctgcatggggatgaccagga |
| | cagtgaggatgaggttctgaccatcccagaggtgaaagttcactcgggcaggggagctggggcag |
| | agagctcccacccagtgagaaacgcacagagcaatgcccttcaggagcgtgaggacgatagggt |
| | ggggctggtcaggggtgagaaggcgaggaaagggaagtccagctctgcacagcagaagacagt |
| | gagctccaccaagctggtgtccttccatgacgacagcgacgaggacctcttacacatctaaGAAT |
| | TC |
| SEQ ID | GTTTAAACGGATCCgccaccATGGATATGAGAGTGCCTGCCCAACTTC |
| NO: 23 | TCGGACTGCTGCTGCTTTGGCTTAGAGGTGCAAGATGCacatgccggctc |
| | agagaatactatgaccagacagctcagatgtgctgcagcaaatgctcgccgggccaacatgcaaa |
| | agtcttctgtaccaagacctcggacaccgtgtgtgactcctgtgaggacagcacatacacccagctct |
| | ggaactgggttcccgagtgcttgagctgtggctcccgctgtagctctgaccaggtggaaactcaagcc |
| | tgcactcgggaacagaaccgcatctgcacctgcaggcccggctggtactgcgcgctgagcaagca |
| | ggaggggtgccggctgtgcgcgccgctgcgcaagtgccgcccgggcttcggcgtggccagaccag |
| | gaactgaaacatcagacgtggtgtgcaagccctgtgccccggggacgttctccaacacgacttcatc |
| | cacggatatttgcaggccccaccagatctgtaacgtggtggccatccctgggaatgcaagcatggat |
| | gcagtctgcacgTCGGGTGGAGGAGGGGGTGGAGGAGGGTCCTACCCAT |
| | ACGATGTTCCAGATTACGCTgcagtcggcgcggtgctcagcctgctgctggtggcgctc |
| | acctgctgcctgctggccctgttgctctacaagaaggagaggagggaaacagtgataagtaagctg |
| | accacttgctgtaggagaagttccaacgtgtcctacaaatactcaaaggtgaataaggaagaagag |
| | acagatgagaatgaaacagagtggctgatggaagagatccagctgcctcctccacggcagggaa |
| | aggaagggcaggagaacggccatattaccaccaagtcagtgaaagccctcagctccctgcatggg |
| | gatgaccaggacagtgaggatgaggttctgaccatcccagaggtgaaagttcactcgggcagggg |
| | agctggggcagagagctcccacccagtgagaaacgcacagagcaatgcccttcaggagcgtgag |
| | gacgatagggtggggctggtcaggggtgagaaggcgaggaaagggaagtccagctctgcacagc |
| | agaagacagtgagctccaccaagctggtgtccttccatgacgacagcgacgaggacctcttacaca |
| | tctaaGAATTC |

Mice chimeric receptor constructs from modified MPRs receptors
(LDCRcd, LDCRsd)

| | |
|---|---|
| SEQ ID | ACCGGTGGATCCgccaccatgttccccttctctggctgctggaggactgaactgttattgttgcta |
| NO: 25 | ctcctggctgtggctgtgagagaatcctggcagata..................TCGGGTGGAGGAG |
| | GGGGTGGAGGAGGGTCCTACCCATACGATGTTCCAGATTACGCTctc |
| | agtgtgggctcgatcttacttgtcatatttgcatcattggttgctgtctatatcattgggggtttcttataccag |
| | cgactggtagtgggggccaagggaatggagcagtttcctcatctggccttctggcaggatcttggcaa |
| | cctagtagctgatggttgtgactttgtgtgccgatccaaaccccgcaatgtgcctgcagcatatcgtgga |
| | gtgggagatgaccagctgggtgaagagtcggaagaaagggatgatcatctgctaccaatgtaaGA |
| | ATTC |
| SEQ ID | ACCGGTGGATCCgccaccatgttccccttctctggctgctggaggactgaactgttattgttgcta |
| NO: 27 | ctcctggctgtggctgtgagagaatcctggcagatagccgatgtgcagctggtggagtctgggggag |
| | ccttggtgcagccgggggggtctctgagactctcctgtgcagcctctggattccccgtcaatcgctatag |
| | tatgaggtggtaccgccaggctccagggaaggagcgcgagtgggtcgcgggtatgagtagtgctgg |
| | tgatcgttcaagttatgaagactccgtgaagggccgattcaccatctccagagacgacgccaggaat |
| | acggtgtatctgcaaatgaacagcctgaaacctgaggacacggccgtgtattactgtaatgtcaatgt |
| | gggctttgagtactggggccaggggacccaggtcaccgtctcctcaTCGGGTGGAGGAGG |
| | GGGTGGAGGAGGGTCCTACCCATACGATGTTCCAGATTACGCTctcag |
| | tgtgggctcgatcttacttgtcatatttgcatcattggttgctgtctatatcattgggggtttcttataccagcg |
| | actggtagtgggggccaagggaatggagcagtttcctcatctggccttctggcaggatcttggcaacct |
| | agtagctgatggttgtgactttgtgtgccgatccaaaccccgcaatgtgcctgcagcatatcgtggagtg |
| | ggagatgaccagctgggtgaagagtcggaagaaagggatgatcatctgctaccaatgtaaGAAT |
| | TC |
| SEQ ID | ACCGGTGGATCCgccaccATGGATATGAGAGTGCCTGCCCAACTTCTC |
| NO: 29 | GGACTGCTGCTGCTTTGGCTTAGAGGTGCAAGATGC..................TC |
| | GGGTGGAGGAGGGGGTGGAGGAGGGTCCTACCCATACGATGTTCC |
| | AGATTACGCTctcagtgtgggctcgatcttacttgtcatatttgcatcattggttgctgtctatatcatt |
| | gggggtttcttataccagcgactggtagtgggggccaagggaatggagcagtttcctcatctggccttc |
| | tggcaggatcttggcaacctagtagctgatggttgtgactttgtgtgccgatccaaaccccgcaatgtgc |
| | ctgcagcatatcgtggagtgggagatgaccagctgggtgaagagtcggaagaaagggatgatcat |
| | ctgctaccaatgtaaGAATTC |
| SEQ ID | ACCGGTGGATCCgccaccATGGATATGAGAGTGCCTGCCCAACTTCTC |
| NO: 31 | GGACTGCTGCTGCTTTGGCTTAGAGGTGCAAGATGCgccgatgtgcagctg |
| | gtggagtctgggggagccttggtgcagccgggggggtctctgagactctcctgtgcagcctctggattc |
| | cccgtcaatcgctatagtatgaggtggtaccgccaggctccagggaaggagcgcgagtgggtcgcg |
| | ggtatgagtagtgctggtgatcgttcaagttatgaagactccgtgaagggccgattcaccatctccaga |
| | gacgacgccaggaatacggtgtatctgcaaatgaacagcctgaaacctgaggacacggccgtgtat |
| | tactgtaatgtcaatgtgggctttgagtactggggccaggggacccaggtcaccgtctcctcaTCGG |
| | GTGGAGGAGGGGGTGGAGGAGGGTCCTACCCATACGATGTTCCAG |
| | ATTACGCTctcagtgtgggctcgatcttacttgtcatatttgcatcattggttgctgtctatatcattggg |
| | ggtttcttataccagcgactggtagtgggggccaagggaatggagcagtttcctcatctggccttctggc |
| | aggatcttggcaacctagtagctgatggttgtgactttgtgtgccgatccaaaccccgcaatgtgcctgc |
| | agcatatcgtggagtgggagatgaccagctgggtgaagagtcggaagaaagggatgatcatctgct |
| | accaatgtaaGAATTC |
| SEQ ID | ACCGGTGGATCCgccaccatgttccctttctacagctgctggaggactggactgctactactac |
| NO: 33 | tcctggctgtggcagtgagagaatcctggcag.................TCGGGTGGAGGAGGG |
| | GGTGGAGGAGGGTCCTACCCATACGATGTTCCAGATTACGCTctcagtg |
| | tgggttccatcttacttgtcacgtttgcatcactggttgctgtttatgttgttggggggttcctataccagcgac |
| | tggtagtgggagccaaaggaatggagcagtttccccacttagccttctggcaggatcttggcaacctg |
| | gtagcagatggctgtgactttgtctgccgttctaaacctcgaaatgtgcctgcagcatatcgtggtgtgg |
| | gggatgaccagctgggggaggagtcagaagaaagggatgaccatttattaccaatgtaaGAATT |
| | C |
| SEQ ID | ACCGGTGGATCCgccaccatgttccctttctacagctgctggaggactggactgctactactac |
| NO: 35 | tcctggctgtggcagtgagagaatcctggcagacatgccggctcagagaatactatgaccagacag |
| | ctcagatgtgctgcagcaaatgctcgccgggccaacatgcaaaagtcttctgtaccaagacctcgga |
| | caccgtgtgtgactcctgtgaggacagcacatacacccagctctggaactgggttcccgagtgcttga |
| | gctgtggctcccgctgtagctctgaccaggtggaaactcaagcctgcactcgggaacagaaccgcat |
| | ctgcacctgcaggcccggctggtactgcgcgctgagcaagcaggaggggtgccggctgtgcgcgc |
| | cgctgcgcaagtgccgcccgggcttcggcgtggccagaccaggaactgaaacatcagacgtggtg |
| | tgcaagccctgtgccccggggacgttctccaacacgacttcatccacggatatttgcaggccccacca |
| | gatctgtaacgtggtggccatccctgggaatgcaagcatggatgcagtctgcacgTCGGGTGG |
| | AGGAGGGGGTGGAGGAGGGTCCTACCCATACGATGTTCCAGATTAC |
| | GCTctcagtgtgggttccatcttacttgtcacgtttgcatcactggttgctgtttatgttgttggggggttcct |
| | ataccagcgactggtagtgggagccaaaggaatggagcagtttccccacttagccttctggcaggat |
| | cttggcaacctggtagcagatggctgtgactttgtctgccgttctaaacctcgaaatgtgcctgcagcat |
| | atcgtggtgtgggggatgaccagctgggggaggagtcagaagaaagggatgaccatttattaccaa |
| | tgtaaGAATTC |
| SEQ ID | ACCGGTGGATCCgccaccATGGATATGAGAGTGCCTGCCCAACTTCTC |
| NO: 37 | GGACTGCTGCTGCTTTGGCTTAGAGGTGCAAGATGC..................TC |
| | GGGTGGAGGAGGGGGTGGAGGAGGGTCCTACCCATACGATGTTCC |
| | AGATTACGCTctcagtgtgggttccatcttacttgtcacgtttgcatcactggttgctgtttatgttgttg |
| | gggggttcctataccagcgactggtagtgggagccaaaggaatggagcagtttccccacttagccttc |
| | tggcaggatcttggcaacctggtagcagatggctgtgactttgtctgccgttctaaacctcgaaatgtgc |
| | ctgcagcatatcgtggtgtgggggatgaccagctgggggaggagtcagaagaaagggatgaccat |
| | ttattaccaatgtaaGAATTC |
| SEQ ID | ACCGGTGGATCCgccaccATGGATATGAGAGTGCCTGCCCAACTTCTC |
| NO: 39 | GGACTGCTGCTGCTTTGGCTTAGAGGTGCAAGATGCacatgccggctcag |
| | agaatactatgaccagacagctcagatgtgctgcagcaaatgctcgccgggccaacatgcaaaagt |
| | cttctgtaccaagacctcggacaccgtgtgtgactcctgtgaggacagcacatacacccagctctgga |
| | actgggttcccgagtgcttgagctgtggctcccgctgtagctctgaccaggtggaaactcaagcctgc |
| | actcgggaacagaaccgcatctgcacctgcaggcccggctggtactgcgcgctgagcaagcagga |
| | ggggtgccggctgtgcgcgccgctgcgcaagtgccgcccgggcttcggcgtggccagaccaggaa |
| | ctgaaacatcagacgtggtgtgcaagccctgtgccccggggacgttctccaacacgacttcatccac |
| | ggatatttgcaggccccaccagatctgtaacgtggtggccatccctgggaatgcaagcatggatgca |
| | gtctgcacgTCGGGTGGAGGAGGGGGTGGAGGAGGGTCCTACCCATAC |
| | GATGTTCCAGATTACGCTctcagtgtgggttccatcttacttgtcacgtttgcatcactggttg |
| | ctgtttatgttgttggggggttcctataccagcgactggtagtgggagccaaaggaatggagcagtttcc |
| | ccacttagccttctggcaggatottggcaacctggtagcagatggctgtgactttgtctgccgttctaaac |
| | ctcgaaatgtgcctgcagcatatcgtggtgtgggggatgaccagctgggggaggagtcagaagaaa |
| | gggatgaccatttattaccaatgtaa |

TABLE 2
Amino Acid Sequence Chimeric Receptor Constructs

| | |
|---|---|
| SEQ ID NO | Sequence |

chimeric receptor constructs from modified ASGPR1 receptor (LDCRag)

| | |
|---|---|
| SEQ ID NO: 2 | YPYDVPDYAMTKDYQDFQHLDNDNDHHQLRRGPPPTPRLLQRL |
| | CSGSRLLLLSSSLSILLLVVVCVITSSGGGGGGGGS |
| SEQ ID NO: 4 | YPYDVPDYAMTKDYQDFQHLDNDNDHHQLRRGPPPTPRLLQRL |
| | CSGSRLLLLSSSLSILLLVVVCVITSSGGGGGGGGSEFECQISQE |
| | YYDRKAQMCCAKCPPGQYVKHFCNKTSDTVCADCEASMYTQV |
| | WNQFRTCLSCSSSCTTDQVEIRACTKQQNRVCACEAGRYCALK |
| | THSGSCRQCMRLSKCGPGFGVASSRAPNGNVLCKACAPGTFSD |
| | TTSSTDVCRPHRICSILAIPGNASTDAVCA |
| SEQ ID NO: 6 | YPYDVPDYAMTKEYQDLQHLDNEESDHHQLRKGPPPPQPLLQR |
| | LCSGPRLLLLSLGLSLLLLVVVCVIGSSGGGGGGGGSEF |
| SEQ ID NO: 8 | YPYDVPDYAMTKEYQDLQHLDNEESDHHQLRKGPPPPQPLLQR |
| | LCSGPRLLLLSLGLSLLLLVVVCVIGSSGGGGGGGGSEFTCRLRE |
| | YYDQTAQMCCSKCSPGQHAKVFCTKTSDTVCDSCEDSTYTQLW |
| | NWVPECLSCGSRCSSDQVETQACTREQNRICTCRPGWYCALSK |
| | QEGCRLCAPLRKCRPGFGVARPGTETSDVVCKPCAPGTFSNTT |
| | SSTDICRPHQICNVVAIPGNASMDAVCT |

Mice chimeric receptor constructs from modified MPRs receptors
(LDCRci, LDCRsi)

| | |
|---|---|
| SEQ ID NO: 10 | MRAVQLGPVPSGPRVALLPPLLLLLLLAAAGSAQA................... |
| | .....SGGGGGGGGSYPYDVPDYAVGAVLSLLLVALTGCLLALLLHK |
| | KERRETVINKLTSCCRRSSGVSYKYSKVSKEEETDENETEWLME |
| | EIQVPAPRLGKDGQENGHITTKAVKAEALSSLHGDDQDSEDEVL |
| | TVPEVKVHSGRGAEVESSQPLRNPQRKVLKEREGERLGLVRGE |
| | KARKGKFRPGQRKPTAPAKLVSFHDDSDEDLLHI |
| SEQ ID NO: 12 | MRAVQLGPVPSGPRVALLPPLLLLLLLAAAGSAQAADVQLVESG |
| | GALVQPGGSLRLSCAASGFPVNRYSMRWYRQAPGKEREWVAG |
| | MSSAGDRSSYEDSVKGRFTISRDDARNTVYLQMNSLKPEDTAVY |
| | YCNVNVGFEYWGQGTQVTVSSSGGGGGGGGSYPYDVPDYAV |
| | GAVLSLLLVALTGCLLALLLHKKERRETVINKLTSCCRRSSGVSYK |
| | YSKVSKEEETDENETEWLMEEIQVPAPRLGKDGQENGHITTKAV |
| | KAEALSSLHGDDQDSEDEVLTVPEVKVHSGRGAEVESSQPLRN |
| | PQRKVLKEREGERLGLVRGEKARKGKFRPGQRKPTAPAKLVSF |
| | HDDSDEDLLHI |
| SEQ ID NO: 14 | MDMRVPAQLLGLLLLWLRGARC........................SGGGGGGGG |
| | SYPYDVPDYAVGAVLSLLLVALTGCLLALLLHKKERRETVINKLTS |
| | CCRRSSGVSYKYSKVSKEEETDENETEWLMEEIQVPAPRLGKD |
| | GQENGHITTKAVKAEALSSLHGDDQDSEDEVLTVPEVKVHSGRG |
| | AEVESSQPLRNPQRKVLKEREGERLGLVRGEKARKGKFRPGQR |
| | KPTAPAKLVSFHDDSDEDLLHI |
| SEQ ID NO: 16 | MDMRVPAQLLGLLLLWLRGARCADVQLVESGGALVQPGGSLRL |
| | SCAASGFPVNRYSMRWYRQAPGKEREWVAGMSSAGDRSSYE |
| | DSVKGRFTISRDDARNTVYLQMNSLKPEDTAVYYCNVNVGFEY |
| | WGQGTQVTVSSSGGGGGGGGSYPYDVPDYAVGAVLSLLLVALT |
| | GCLLALLLHKKERRETVINKLTSCCRRSSGVSYKYSKVSKEEETD |
| | ENETEWLMEEIQVPAPRLGKDGQENGHITTKAVKAEALSSLHGD |
| | DQDSEDEVLTVPEVKVHSGRGAEVESSQPLRNPQRKVLKEREG |
| | ERLGLVRGEKARKGKFRPGQRKPTAPAKLVSFHDDSDEDLLHI |

Human chimeric receptor constructs from modified MPRs receptors
(LDCRci, LDCRsi)

| | |
|---|---|
| SEQ ID NO: 18 | MGAAAGRSPHLGPAPARRPQRSLLLLQLLLLVAAPGSTQA......... |
| | ...............SGGGGGGGGSYPYDVPDYAAVGAVLSLLLVALTCCL |
| | LALLLYKKERRETVISKLTTCCRRSSNVSYKYSKVNKEEETDENE |
| | TEWLMEEIQLPPPRQGKEGQENGHITTKSVKALSSLHGDDQDSE |
| | DEVLTIPEVKVHSGRGAGAESSHPVRNAQSNALQEREDDRVGLV |
| | RGEKARKGKSSSAQQKTVSSTKLVSFHDDSDEDLLHI |
| SEQ ID NO: 20 | MGAAAGRSPHLGPAPARRPQRSLLLLQLLLLVAAPGSTQATCRL |
| | REYYDQTAQMCCSKCSPGQHAKVFCTKTSDTVCDSCEDSTYTQ |
| | LWNWVPECLSCGSRCSSDQVETQACTREQNRICTCRPGWYCA |
| | LSKQEGCRLCAPLRKCRPGFGVARPGTETSDVVCKPCAPGTFS |
| | NTTSSTDICRPHQICNVVAIPGNASMDAVCTSGGGGGGGGSYPY |
| | DVPDYAAVGAVLSLLLVALTCCLLALLLYKKERRETVISKLTTCCR |
| | RSSNVSYKYSKVNKEEETDENETEWLMEEIQLPPPRQGKEGQE |
| | NGHITTKSVKALSSLHGDDQDSEDEVLTIPEVKVHSGRGAGAES |
| | SHPVRNAQSNALQEREDDRVGLVRGEKARKGKSSSAQQKTVSS |
| | TKLVSFHDDSDEDLLHI |
| SEQ ID NO: 22 | MDMRVPAQLLGLLLLWLRGARC.....................SGGGGGGGGS |
| | YPYDVPDYAAVGAVLSLLLVALTCCLLALLLYKKERRETVISKLTT |
| | CCRRSSNVSYKYSKVNKEEETDENETEWLMEEIQLPPPRQGKE |
| | GQENGHITTKSVKALSSLHGDDQDSEDEVLTIPEVKVHSGRGAG |
| | AESSHPVRNAQSNALQEREDDRVGLVRGEKARKGKSSSAQQKT |
| | VSSTKLVSFHDDSDEDLLHI |
| SEQ ID NO: 24 | MDMRVPAQLLGLLLLWLRGARCTCRLREYYDQTAQMCCSKCSP |
| | GQHAKVFCTKTSDTVCDSCEDSTYTQLWNWVPECLSCGSRCSS |
| | DQVETQACTREQNRICTCRPGWYCALSKQEGCRLCAPLRKCRP |
| | GFGVARPGTETSDVVCKPCAPGTFSNTTSSTDICRPHQICNVVAI |
| | PGNASMDAVCTSGGGGGGGGSYPYDVPDYAAVGAVLSLLLVAL |
| | TCCLLALLLYKKERRETVISKLTTCCRRSSNVSYKYSKVNKEEET |
| | DENETEWLMEEIQLPPPRQGKEGQENGHITTKSVKALSSLHGDD |
| | QDSEDEVLTIPEVKVHSGRGAGAESSHPVRNAQSNALQEREDD |
| | RVGLVRGEKARKGKSSSAQQKTVSSTKLVSFHDDSDEDLLHI |

Mice chimeric receptor constructs from modified MPRs receptors
(LDCRcd, LDCRsd)

| | |
|---|---|
| SEQ ID NO: 26 | MFPFSGCWRTELLLLLLLAVAVRESWQI.....................SGGGGG |
| | GGGSYPYDVPDYALSVGSILLVIFASLVAVYIIGGFLYQRLVVGAK |
| | GMEQFPHLAFWQDLGNLVADGCDFVCRSKPRNVPAAYRGVGD |
| | DQLGEESEERDDHLLPM |
| SEQ ID NO: 28 | MFPFSGCWRTELLLLLLLAVAVRESWQIADVQLVESGGALVQPG |
| | GSLRLSCAASGFPVNRYSMRWYRQAPGKEREWVAGMSSAGDR |
| | SSYEDSVKGRFTISRDDARNTVYLQMNSLKPEDTAVYYCNVNVG |
| | FEYWGQGTQVTVSSSGGGGGGGGSYPYDVPDYALSVGSILLVIF |
| | ASLVAVYIIGGFLYQRLVVGAKGMEQFPHLAFWQDLGNLVADGC |
| | DFVCRSKPRNVPAAYRGVGDDQLGEESEERDDHLLPM |
| SEQ ID NO: 30 | MDMRVPAQLLGLLLLWLRGARC.....................SGGGGGGGGS |
| | YPYDVPDYALSVGSILLVIFASLVAVYIIGGFLYQRLVVGAKGMEQ |
| | FPHLAFWQDLGNLVADGCDFVCRSKPRNVPAAYRGVGDDQLGE |
| | ESEERDDHLLPM |
| SEQ ID NO: 32 | MDMRVPAQLLGLLLLWLRGARCADVQLVESGGALVQPGGSLRL |
| | SCAASGFPVNRYSMRWYRQAPGKEREWVAGMSSAGDRSSYE |
| | DSVKGRFTISRDDARNTVYLQMNSLKPEDTAVYYCNVNVGFEY |
| | WGQGTQVTVSSSGGGGGGGGSYPYDVPDYALSVGSILLVIFASL |
| | VAVYIIGGFLYQRLVVGAKGMEQFPHLAFWQDLGNLVADGCDFV |
| | CRSKPRNVPAAYRGVGDDQLGEESEERDDHLLPM |
| SEQ ID NO: 34 | MFPFYSCWRTGLLLLLLAVAVRESWQSGGGGGGGGSYPYDVP |
| | DYALSVGSILLVTFASLVAVYVVGGFLYQRLVVGAKGMEQFPHLA |
| | FWQDLGNLVADGCDFVCRSKPRNVPAAYRGVGDDQLGEESEE |
| | RDDHLLPM |
| SEQ ID NO: 36 | MFPFYSCWRTGLLLLLLAVAVRESWQTCRLREYYDQTAQMCCS |
| | KCSPGQHAKVFCTKTSDTVCDSCEDSTYTQLWNWVPECLSCGS |
| | RCSSDQVETQACTREQNRICTCRPGWYCALSKQEGCRLCAPLR |
| | KCRPGFGVARPGTETSDVVCKPCAPGTFSNTTSSTDICRPHQIC |
| | NVVAIPGNASMDAVCTSGGGGGGGGSYPYDVPDYALSVGSILLV |
| | TFASLVAVYVVGGFLYQRLVVGAKGMEQFPHLAFWQDLGNLVA |
| | DGCDFVCRSKPRNVPAAYRGVGDDQLGEESEERDDHLLPM |
| SEQ ID NO: 38 | MDMRVPAQLLGLLLLWLRGARC...................SGGGGGGGGSY |
| | PYDVPDYALSVGSILLVTFASLVAVYVVGGFLYQRLVVGAKGME |
| | QFPHLAFWQDLGNLVADGCDFVCRSKPRNVPAAYRGVGDDQL |
| | GEESEERDDHLLPM |
| SEQ ID NO: 40 | MDMRVPAQLLGLLLLWLRGARCTCRLREYYDQTAQMCCSKCSP |
| | GQHAKVFCTKTSDTVCDSCEDSTYTQLWNWVPECLSCGSRCSS |
| | DQVETQACTREQNRICTCRPGWYCALSKQEGCRLCAPLRKCRP |
| | GFGVARPGTETSDVVCKPCAPGTFSNTTSSTDICRPHQICNVVAI |
| | PGNASMDAVCTSGGGGGGGGSYPYDVPDYALSVGSILLVTFAS |
| | LVAVYVVGGFLYQRLVVGAKGMEQFPHLAFWQDLGNLVADGCD |
| | FVCRSKPRNVPAAYRGVGDDQLGEESEERDDHLLPM |

What is claimed is:

1. A composition for degrading a target protein in a patient, the composition comprising a lysosomal degrading chimeric receptor (LDCR), the LDCR comprising:
a. an engineered mannose 6-phosphate receptor (MPR), the engineered MPR comprising an MPR with a targeting moiety fused at least one of a transmembrane domain, a cytoplasmic domain, and any combination thereof; or
b. an engineered asialoglycoprotein receptor (ASGPR), the engineered ASGPR comprising an ASGPR with the targeting moiety fused at least one of an extracellular stalk region and a carbohydrate recognition binding domain, and any combination thereof;
wherein the targeting moiety is configured to bind to the target protein.

2. The composition of claim 1, wherein the targeting moiety comprises a nanobody targeting the target protein or an autoantibody epitope sequence targeting the target protein.

3. The composition of claim 1, further comprising the LDCR incorporated into an adeno-associated virus or a chimeric antigen receptor T-cell therapy.

4. The composition of claim 1, wherein the target protein is selected from one of a ligand-binding domain, an autoantigen peptide sequence encoding an autoantibody, a single-chain variable fragment (scFV), or a nanobody.

5. The composition of claim 4, wherein the ligand-binding domain is configured to selectively bind to a chemokine or a cytokine.

6. The composition of claim 4, wherein the autoantigen encoded by the autoantigen peptide sequence is configured to selectively bind to an autoantibody.

7. The composition of claim 4, wherein the nanobody is configured to selectively bind to a chemokine, a cytokine, or an extracellular therapeutic pathological proteins.

8. The composition of claim 1, wherein the LDCR is encoded by a nucleotide sequence selected from the group consisting of SEQ ID NOS. 1, 3, 5, 7, 9, 11, 13, 15, 17, 19, 21, 23, 25, 27, 29, 31, 33, 35, 37, or 39.

9. The composition of claim 1, wherein the LDCR comprises a peptide sequence selected from the group consisting of SEQ ID NOS. 2, 4, 6, 8, 10, 12, 14, 16, 18, 20, 22, 24, 26, 28, 30, 32, 34, 36, 38, or 40.

10. A method of treating a disease or disorder in a patient by targeting a secreted protein for degradation via a lysosome, the method comprising administering a composition comprising a therapeutically effective amount of a lysosomal degrading chimeric receptor (LDCR) to the patient the LDCR comprising:
a. an engineered mannose 6-phosphate receptor (MPR), the engineered MPR comprising an MPR with a targeting moiety fused at least one of a transmembrane domain, a cytoplasmic domain, and any combination thereof; or
b. an engineered asialoglycoprotein receptor (ASGPR), the engineered ASGPR comprising an ASGPR with the targeting moiety fused at least one of an extracellular stalk region and a carbohydrate recognition binding domain, and any combination thereof;
wherein the targeting moiety is configured to bind to the target protein.

11. The method of claim 10, the method further comprising administering the therapeutically effective amount of the LDCR using a method selected from adeno-associated virus (AAV) and chimeric antigen receptor T-cell therapy.

12. The method of claim 10, wherein the disease is an autoimmune disease or Alzheimer's Disease (AD).

13. The method of claim 10, wherein the secreted protein targeted by the nanobody is selected from an epitope from a tau-5 protein, an epitope from an Apoe4 protein, or an epitope from a myelin-based protein.

14. The method of claim 10, wherein the targeting moiety comprises a nanobody targeting the target protein or an autoantibody epitope sequence targeting the target protein.

15. The method of claim 10, wherein the target protein is selected from one of a ligand-binding domain, an autoantigen peptide sequence encoding an autoantibody, a single-chain variable fragment (scFV), or a nanobody.

16. The method of claim 15, wherein the ligand-binding domain is configured to selectively bind to a chemokine or a cytokine.

17. The method of claim 15, wherein the autoantigen encoded by the autoantigen peptide sequence is configured to selectively bind to an autoantibody.

18. The method of claim 10, wherein the LDCR is encoded by a nucleotide sequence selected from the group consisting of SEQ ID NOS. 1, 3, 5, 7, 9, 11, 13, 15, 17, 19, 21, 23, 25, 27, 29, 31, 33, 35, 37, or 39.

19. The method of claim 10, wherein the LDCR comprises a peptide sequence selected from the group consisting of SEQ ID NOS. 2, 4, 6, 8, 10, 12, 14, 16, 18, 20, 22, 24, 26, 28, 30, 32, 34, 36, 38, or 40.


SEQUENCE LISTING (40 sequences)

SEQ ID NO: 1 - DNA, 272 bp, synthetic construct
  source 1..272: mol_type = other DNA
ctagctagcgcggccgctccgccaccatgtacccatacgatgttccagattacgctatgacaaaggattatcaagatttccagcacctggacaatgataatgaccatcatcaactccggagagggccgcctcccactccacggctcttgcagcgactctgctctggatcccgcctcctcctgctctcctcgagcctcagcattctgttgctggtggttgtctgtgtgatcacatcctcgggtggaggagggggtggaggagggtccgaattc

SEQ ID NO: 2 - protein, 79 aa, synthetic construct
YPYDVPDYAMTKDYQDFQHLDNDNDHHQLRRGPPPTPRLLQRLCSGSRLLLLSSSLSILLLVVVCVITSSGGGGGGGGS

SEQ ID NO: 3 - DNA, 782 bp, synthetic construct
  source 1..782: mol_type = other DNA
gtagctagcgcggccgctccgccaccatgtacccatacgatgttccagattacgctatgacaaaggattatcaagatttccagcacctggacaatgataatgaccatcatcaactccggagagggccgcctcccactccacggctcttgcagcgactctgctctggatcccgcctcctcctgctctcctcgagcctcagcattctgttgctggtggttgtctgtgtgatcacatcctcgggtggaggagggggtggaggagggtccgaattcgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgccgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgtaaaagcttgtcgac

SEQ ID NO: 4 - protein, 246 aa, synthetic construct
YPYDVPDYAMTKDYQDFQHLDNDNDHHQLRRGPPPTPRLLQRLCSGSRLLLLSSSLSILLLVVVCVITSSGGGGGGGGSEFECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCA

SEQ ID NO: 5 - DNA, 275 bp, synthetic construct
  source 1..275: mol_type = other DNA
ctagctagcgcggccgctccgccaccatgtacccatacgatgttccagattacgctatgaccaaggagtatcaagaccttcagcatctggacaatgaggagagtgaccaccatcagctcagaaaagggccacctcctccccagcccctcctgcagcgtctctgctccggacctcgcctcctcctgctctccctgggcctcagcctcctgctgcttgtggttgtctgtgtgatcggatcctcgggtggaggagggggtggaggagggtccgaattc

SEQ ID NO: 6 - protein, 82 aa, synthetic construct
YPYDVPDYAMTKEYQDLQHLDNEESDHHQLRKGPPPPQPLLQRLCSGPRLLLLSLGLSLLLLVVVCVIGSSGGGGGGGGSEF

SEQ ID NO: 7 - DNA, 779 bp, synthetic construct
  source 1..779: mol_type = other DNA
ctagctagcgcggccgctccgccaccatgtacccatacgatgttccagattacgctatgaccaaggagtatcaagaccttcagcatctggacaatgaggagagtgaccaccatcagctcagaaaagggccacctcctccccagcccctcctgcagcgtctctgctccggacctcgcctcctcctgctctccctgggcctcagcctcctgctgcttgtggttgtctgtgtgatcggatcctcgggtggaggagggggtggaggagggtccgaattcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtaaaagcttgtcgac

SEQ ID NO: 8 - protein, 245 aa, synthetic construct
YPYDVPDYAMTKEYQDLQHLDNEESDHHQLRKGPPPPQPLLQRLCSGPRLLLLSLGLSLLLLVVVCVIGSSGGGGGGGGSEFTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCT

SEQ ID NO: 9 - DNA, 755 bp, synthetic construct
  source 1..755: mol_type = other DNA
gtttaaacggatccgccaccatgcgggccgttcagctgggaccggtgccatccgggccacgcgtcgcgctcctgccaccactcctgctgctgctgctcctggcagcagcaggctccgcacaggcctcgggtggaggagggggtggaggagggtcctacccatacgatgttccagattacgctgttggggcagtcctcagtcttctcctggtggcgctgactggctgcttgctggccttactgctgcataagaaggagagaagggaaactgtgataaacaagctgaccagctgctgcagaagaagctcgggcgtgtcctacaagtactcaaaggtcagcaaggaggaggagacagatgagaatgagacagaatggctgatggaagagatccaggtgccagccccaagactaggcaaggacgggcaagaaaatggccatatcaccaccaaggcggtgaaagcagaagctctctcgtccctgcatggggacgaccaggacagtgaggatgaggtcctgaccgttccagaggtgaaagttcactcaggccgaggagcggaggtggaaagctcacagcccctgagaaacccacagcgcaaggtcctgaaggaaagggagggtgagaggctggggctggtccgaggtgagaaggccagaaaagggaagttcagacccggacagcgcaagccaacagcccctgccaagctggtgtccttccatgatgacagcgacgaagacctcttacacatctaagaattc

SEQ ID NO: 10 - protein, 242 aa, synthetic construct
MRAVQLGPVPSGPRVALLPPLLLLLLLAAAGSAQASGGGGGGGGSYPYDVPDYAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 11 - DNA, 1103 bp, synthetic construct
  source 1..1103: mol_type = other DNA
gtttaaacggatccgccaccatgcgggccgttcagctgggaccggtgccatccgggccacgcgtcgcgctcctgccaccactcctgctgctgctgctcctggcagcagcaggctccgcacaggccgccgatgtgcagctggtggagtctgggggagccttggtgcagccgggggggtctctgagactctcctgtgcagcctctggattccccgtcaatcgctatagtatgaggtggtaccgccaggctccagggaaggagcgcgagtgggtcgcgggtatgagtagtgctggtgatcgttcaagttatgaagactccgtgaagggccgattcaccatctccagagacgacgccaggaatacggtgtatctgcaaatgaacagcctgaaacctgaggacacggccgtgtattactgtaatgtcaatgtgggctttgagtactggggccaggggacccaggtcaccgtctcctcatcgggtggaggagggggtggaggagggtcctacccatacgatgttccagattacgctgttggggcagtcctcagtcttctcctggtggcgctgactggctgcttgctggccttactgctgcataagaaggagagaagggaaactgtgataaacaagctgaccagctgctgcagaagaagctcgggcgtgtcctacaagtactcaaaggtcagcaaggaggaggagacagatgagaatgagacagaatggctgatggaagagatccaggtgccagccccaagactaggcaaggacgggcaagaaaatggccatatcaccaccaaggcggtgaaagcagaagctctctcgtccctgcatggggacgaccaggacagtgaggatgaggtcctgaccgttccagaggtgaaagttcactcaggccgaggagcggaggtggaaagctcacagcccctgagaaacccacagcgcaaggtcctgaaggaaagggagggtgagaggctggggctggtccgaggtgagaaggccagaaaagggaagttcagacccggacagcgcaagccaacagcccctgccaagctggtgtccttccatgatgacagcgacgaagacctcttacacatctaagaattc

SEQ ID NO: 12 - protein, 358 aa, synthetic construct
MRAVQLGPVPSGPRVALLPPLLLLLLLAAAGSAQAADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSSGGGGGGGGSYPYDVPDYAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 13 - DNA, 716 bp, synthetic construct
  source 1..716: mol_type = other DNA
gtttaaacggatccgccaccatggatatgagagtgcctgcccaacttctcggactgctgctgctttggcttagaggtgcaagatgctcgggtggaggagggggtggaggagggtcctacccatacgatgttccagattacgctgttggggcagtcctcagtcttctcctggtggcgctgactggctgcttgctggccttactgctgcataagaaggagagaagggaaactgtgataaacaagctgaccagctgctgcagaagaagctcgggcgtgtcctacaagtactcaaaggtcagcaaggaggaggagacagatgagaatgagacagaatggctgatggaagagatccaggtgccagccccaagactaggcaaggacgggcaagaaaatggccatatcaccaccaaggcggtgaaagcagaagctctctcgtccctgcatggggacgaccaggacagtgaggatgaggtcctgaccgttccagaggtgaaagttcactcaggccgaggagcggaggtggaaagctcacagcccctgagaaacccacagcgcaaggtcctgaaggaaagggagggtgagaggctggggctggtccgaggtgagaaggccagaaaagggaagttcagacccggacagcgcaagccaacagcccctgccaagctggtgtccttccatgatgacagcgacgaagacctcttacacatctaagaattc

SEQ ID NO: 14 - protein, 229 aa, synthetic construct
MDMRVPAQLLGLLLLWLRGARCSGGGGGGGGSYPYDVPDYAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 15 - DNA, 1064 bp, synthetic construct
  source 1..1064: mol_type = other DNA
gtttaaacggatccgccaccatggatatgagagtgcctgcccaacttctcggactgctgctgctttggcttagaggtgcaagatgcgccgatgtgcagctggtggagtctgggggagccttggtgcagccgggggggtctctgagactctcctgtgcagcctctggattccccgtcaatcgctatagtatgaggtggtaccgccaggctccagggaaggagcgcgagtgggtcgcgggtatgagtagtgctggtgatcgttcaagttatgaagactccgtgaagggccgattcaccatctccagagacgacgccaggaatacggtgtatctgcaaatgaacagcctgaaacctgaggacacggccgtgtattactgtaatgtcaatgtgggctttgagtactggggccaggggacccaggtcaccgtctcctcatcgggtggaggagggggtggaggagggtcctacccatacgatgttccagattacgctgttggggcagtcctcagtcttctcctggtggcgctgactggctgcttgctggccttactgctgcataagaaggagagaagggaaactgtgataaacaagctgaccagctgctgcagaagaagctcgggcgtgtcctacaagtactcaaaggtcagcaaggaggaggagacagatgagaatgagacagaatggctgatggaagagatccaggtgccagccccaagactaggcaaggacgggcaagaaaatggccatatcaccaccaaggcggtgaaagcagaagctctctcgtccctgcatggggacgaccaggacagtgaggatgaggtcctgaccgttccagaggtgaaagttcactcaggccgaggagcggaggtggaaagctcacagcccctgagaaacccacagcgcaaggtcctgaaggaaagggagggtgagaggctggggctggtccgaggtgagaaggccagaaaagggaagttcagacccggacagcgcaagccaacagcccctgccaagctggtgtccttccatgatgacagcgacgaagacctcttacacatctaagaattc

SEQ ID NO: 16 - protein, 345 aa, synthetic construct
MDMRVPAQLLGLLLLWLRGARCADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSSGGGGGGGGSYPYDVPDYAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 17 - DNA, 767 bp, synthetic construct
  source 1..767: mol_type = other DNA
gtttaaacggatccgccaccatgggggccgccgccggccggagcccccacctggggcccgcgcccgcccgccgcccgcagcgctctctgctcctgctgcagctgctgctgctcgtcgctgccccggggtccacgcaggcctcgggtggaggagggggtggaggagggtcctacccatacgatgttccagattacgctgcagtcggcgcggtgctcagcctgctgctggtggcgctcacctgctgcctgctggccctgttgctctacaagaaggagaggagggaaacagtgataagtaagctgaccacttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtgaggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctaagaattc

SEQ ID NO: 18 - protein, 246 aa, synthetic construct
MGAAAGRSPHLGPAPARRPQRSLLLLQLLLLVAAPGSTQASGGGGGGGGSYPYDVPDYAAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 19 - DNA, 1256 bp, synthetic construct
  source 1..1256: mol_type = other DNA
gtttaaacggatccgccaccatgggggccgccgccggccggagcccccacctggggcccgcgcccgcccgccgcccgcagcgctctctgctcctgctgcagctgctgctgctcgtcgctgccccggggtccacgcaggccacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtcgggtggaggagggggtggaggagggtcctacccatacgatgttccagattacgctgcagtcggcgcggtgctcagcctgctgctggtggcgctcacctgctgcctgctggccctgttgctctacaagaaggagaggagggaaacagtgataagtaagctgaccacttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtgaggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctaagaattc

SEQ ID NO: 20 - protein, 409 aa, synthetic construct
MGAAAGRSPHLGPAPARRPQRSLLLLQLLLLVAAPGSTQATCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSGGGGGGGGSYPYDVPDYAAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 21 - DNA, 713 bp, synthetic construct
  source 1..713: mol_type = other DNA
gtttaaacggatccgccaccatggatatgagagtgcctgcccaacttctcggactgctgctgctttggcttagaggtgcaagatgctcgggtggaggagggggtggaggagggtcctacccatacgatgttccagattacgctgcagtcggcgcggtgctcagcctgctgctggtggcgctcacctgctgcctgctggccctgttgctctacaagaaggagaggagggaaacagtgataagtaagctgaccacttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtgaggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctaagaattc

SEQ ID NO: 22 - protein, 228 aa, synthetic construct
MDMRVPAQLLGLLLLWLRGARCSGGGGGGGGSYPYDVPDYAAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 23 - DNA, 1202 bp, synthetic construct
  source 1..1202: mol_type = other DNA
gtttaaacggatccgccaccatggatatgagagtgcctgcccaacttctcggactgctgctgctttggcttagaggtgcaagatgcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtcgggtggaggagggggtggaggagggtcctacccatacgatgttccagattacgctgcagtcggcgcggtgctcagcctgctgctggtggcgctcacctgctgcctgctggccctgttgctctacaagaaggagaggagggaaacagtgataagtaagctgaccacttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtgaggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctaagaattc

SEQ ID NO: 24 - protein, 391 aa, synthetic construct
MDMRVPAQLLGLLLLWLRGARCTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSGGGGGGGGSYPYDVPDYAAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 25 - DNA, 444 bp, synthetic construct
  source 1..444: mol_type = other DNA
accggtggatccgccaccatgttccccttctctggctgctggaggactgaactgttattgttgctactcctggctgtggctgtgagagaatcctggcagatatcgggtggaggagggggtggaggagggtcctacccatacgatgttccagattacgctctcagtgtgggctcgatcttacttgtcatatttgcatcattggttgctgtctatatcattgggggtttcttataccagcgactggtagtgggggccaagggaatggagcagtttcctcatctggccttctggcaggatcttggcaacctagtagctgatggttgtgactttgtgtgccgatccaaaccccgcaatgtgcctgcagcatatcgtggagtgggagatgaccagctgggtgaagagtcggaagaaagggatgatcatctgctaccaatgtaagaattc

SEQ ID NO: 26 - protein, 139 aa, synthetic construct
MFPFSGCWRTELLLLLLLAVAVRESWQISGGGGGGGGSYPYDVPDYALSVGSILLVIFASLVAVYIIGGFLYQRLVVGAKGMEQFPHLAFWQDLGNLVADGCDFVCRSKPRNVPAAYRGVGDDQLGEESEERDDHLLPM

SEQ ID NO: 27 - DNA, 792 bp, synthetic construct
  source 1..792: mol_type = other DNA
accggtggatccgccaccatgttccccttctctggctgctggaggactgaactgttattgttgctactcctggctgtggctgtgagagaatcctggcagatagccgatgtgcagctggtggagtctgggggagccttggtgcagccgggggggtctctgagactctcctgtgcagcctctggattccccgtcaatcgctatagtatgaggtggtaccgccaggctccagggaaggagcgcgagtgggtcgcgggtatgagtagtgctggtgatcgttcaagttatgaagactccgtgaagggccgattcaccatctccagagacgacgccaggaatacggtgtatctgcaaatgaacagcctgaaacctgaggacacggccgtgtattactgtaatgtcaatgtgggctttgagtactggggccaggggacccaggtcaccgtctcctcatcgggtggaggagggggtggaggagggtcctacccatacgatgttccagattacgctctcagtgtgggctcgatcttacttgtcatatttgcatcattggttgctgtctatatcattgggggtttcttataccagcgactggtagtgggggccaagggaatggagcagtttcctcatctggccttctggcaggatcttggcaacctagtagctgatggttgtgactttgtgtgccgatccaaaccccgcaatgtgcctgcagcatatcgtggagtgggagatgaccagctgggtgaagagtcggaagaaagggatgatcatctgctaccaatgtaagaattc

SEQ ID NO: 28 - protein, 255 aa, synthetic construct
MFPFSGCWRTELLLLLLLAVAVRESWQIADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSSGGGGGGGGSYPYDVPDYALSVGSILLVIFASLVAVYIIGGFLYQRLVVGAKGMEQFPHLAFWQDLGNLVADGCDFVCRSKPRNVPAAYRGVGDDQLGEESEERDDHLLPM

SEQ ID NO: 29 - DNA, 426 bp, synthetic construct
  source 1..426: mol_type = other DNA
accggtggatccgccaccatggatatgagagtgcctgcccaacttctcggactgctgctgctttggcttagaggtgcaagatgctcgggtggaggagggggtggaggagggtcctacccatacgatgttccagattacgctctcagtgtgggctcgatcttacttgtcatatttgcatcattggttgctgtctatatcattgggggtttcttataccagcgactggtagtgggggccaagggaatggagcagtttcctcatctggccttctggcaggatcttggcaacctagtagctgatggttgtgactttgtgtgccgatccaaaccccgcaatgtgcctgcagcatatcgtggagtgggagatgaccagctgggtgaagagtcggaagaaagggatgatcatctgctaccaatgtaagaattc

SEQ ID NO: 30 - protein, 133 aa, synthetic construct
MDMRVPAQLLGLLLLWLRGARCSGGGGGGGGSYPYDVPDYALSVGSILLVIFASLVAVYIIGGFLYQRLVVGAKGMEQFPHLAFWQDLGNLVADGCDFVCRSKPRNVPAAYRGVGDDQLGEESEERDDHLLPM

SEQ ID NO: 31 - DNA, 774 bp, synthetic construct
  source 1..774: mol_type = other DNA
accggtggatccgccaccatggatatgagagtgcctgcccaacttctcggactgctgctgctttggcttagaggtgcaagatgcgccgatgtgcagctggtggagtctgggggagccttggtgcagccgggggggtctctgagactctcctgtgcagcctctggattccccgtcaatcgctatagtatgaggtggtaccgccaggctccagggaaggagcgcgagtgggtcgcgggtatgagtagtgctggtgatcgttcaagttatgaagactccgtgaagggccgattcaccatctccagagacgacgccaggaatacggtgtatctgcaaatgaacagcctgaaacctgaggacacggccgtgtattactgtaatgtcaatgtgggctttgagtactggggccaggggacccaggtcaccgtctcctcatcgggtggaggagggggtggaggagggtcctacccatacgatgttccagattacgctctcagtgtgggctcgatcttacttgtcatatttgcatcattggttgctgtctatatcattgggggtttcttataccagcgactggtagtgggggccaagggaatggagcagtttcctcatctggccttctggcaggatcttggcaacctagtagctgatggttgtgactttgtgtgccgatccaaaccccgcaatgtgcctgcagcatatcgtggagtgggagatgaccagctgggtgaagagtcggaagaaagggatgatcatctgctaccaatgtaagaattc

SEQ ID NO: 32 - protein, 249 aa, synthetic construct
MDMRVPAQLLGLLLLWLRGARCADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSSGGGGGGGGSYPYDVPDYALSVGSILLVIFASLVAVYIIGGFLYQRLVVGAKGMEQFPHLAFWQDLGNLVADGCDFVCRSKPRNVPAAYRGVGDDQLGEESEERDDHLLPM

SEQ ID NO: 33 - DNA, 438 bp, synthetic construct
  source 1..438: mol_type = other DNA
accggtggatccgccaccatgttccctttctacagctgctggaggactggactgctactactactcctggctgtggcagtgagagaatcctggcagtcgggtggaggagggggtggaggagggtcctacccatacgatgttccagattacgctctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatgtaagaattc

SEQ ID NO: 34 - protein, 137 aa, synthetic construct
MFPFYSCWRTGLLLLLLAVAVRESWQSGGGGGGGGSYPYDVPDYALSVGSILLVTFASLVAVYVVGGFLYQRLVVGAKGMEQFPHLAFWQDLGNLVADGCDFVCRSKPRNVPAAYRGVGDDQLGEESEERDDHLLPM

SEQ ID NO: 35 - DNA, 927 bp, synthetic construct
  source 1..927: mol_type = other DNA
accggtggatccgccaccatgttccctttctacagctgctggaggactggactgctactactactcctggctgtggcagtgagagaatcctggcagacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtcgggtggaggagggggtggaggagggtcctacccatacgatgttccagattacgctctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatgtaagaattc

SEQ ID NO: 36 - protein, 300 aa, synthetic construct
MFPFYSCWRTGLLLLLLAVAVRESWQTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSGGGGGGGGSYPYDVPDYALSVGSILLVTFASLVAVYVVGGFLYQRLVVGAKGMEQFPHLAFWQDLGNLVADGCDFVCRSKPRNVPAAYRGVGDDQLGEESEERDDHLLPM

SEQ ID NO: 37 - DNA, 426 bp, synthetic construct
  source 1..426: mol_type = other DNA
accggtggatccgccaccatggatatgagagtgcctgcccaacttctcggactgctgctgctttggcttagaggtgcaagatgctcgggtggaggagggggtggaggagggtcctacccatacgatgttccagattacgctctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatgtaagaattc

SEQ ID NO: 38 - protein, 133 aa, synthetic construct
MDMRVPAQLLGLLLLWLRGARCSGGGGGGGGSYPYDVPDYALSVGSILLVTFASLVAVYVVGGFLYQRLVVGAKGMEQFPHLAFWQDLGNLVADGCDFVCRSKPRNVPAAYRGVGDDQLGEESEERDDHLLPM

SEQ ID NO: 39 - DNA, 909 bp, synthetic construct
  source 1..909: mol_type = other DNA
accggtggatccgccaccatggatatgagagtgcctgcccaacttctcggactgctgctgctttggcttagaggtgcaagatgcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtcgggtggaggagggggtggaggagggtcctacccatacgatgttccagattacgctctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatgtaa

SEQ ID NO: 40 - protein, 296 aa, synthetic construct
MDMRVPAQLLGLLLLWLRGARCTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSGGGGGGGGSYPYDVPDYALSVGSILLVTFASLVAVYVVGGFLYQRLVVGAKGMEQFPHLAFWQDLGNLVADGCDFVCRSKPRNVPAAYRGVGDDQLGEESEERDDHLLPM